(19) United States
(12) Patent Application Publication
(10) Pub. No.: US 2017/0121633 A1
(43) Pub. Date: May 4, 2017

(54) Fragrance Compositions Comprising Ionic Liquids

(71) Applicant: The Procter & Gamble Company, Cincinnati, OH (US)

(72) Inventors: Lynette Anne Makins HOLLAND, Abbots Langley (GB); Henry Charles Reginald FOVARGUE, London (GB); Kenneth Richard SEDDON, Belfast (GB); Harambage Quintus Nimal GUNARATNE, Belfast (GB); Alberto Vaca PUGA, Belfast (GB); Federico Maria FERRERO VALLANA, Belfast (GB)

(21) Appl. No.: 15/336,894

(22) Filed: Oct. 28, 2016

Related U.S. Application Data
(60) Provisional application No. 62/247,738, filed on Oct. 28, 2015.

(51) Int. Cl. C11B 9/00; A61K 8/41; A61K 8/34; A61K 8/49; A61Q 15/00; A61Q 19/00

(52) U.S. Cl. CPC C11B 9/0061; A61Q 15/00; A61Q 19/002; C11B 9/0096; C11B 9/0007; A61K 8/34; A61K 8/4946; A61K 8/416; A61K 2800/30

(57) ABSTRACT

The present invention relates to a fragrance composition comprising ionic liquids for delayed evaporation of the perfume raw materials. The invention also relates to methods of use of the fragrance compositions for perfuming suitable substrates, particularly skin and hair.

### FIELD OF THE INVENTION

The present invention relates to fragrance compositions comprising ionic liquids. In particular, the fragrance compositions of the present invention have delayed evaporation of the fragrance component.

### BACKGROUND OF THE INVENTION

Perfume raw materials (PRMs) have their own inherent volatility as determined in part by their molecular weight (i.e., size) and in part by the interaction with their surroundings (i.e., ability to hydrogen bond with other PRMs or solvents). The volatility of the PRMs can span a wide range and impact the evaporation rate and/or release of the fragrance components from a composition into the headspace (and thus becoming olfactorily noticeable). For example, low volatile PRMs, as characterized by having a vapour pressure less than about 0.001 Torr (<0.00013 kPa) at 25° C., may smell sweet, musky and woody, and can last for several days. Alternatively, the highly volatile

PRMs, represented by those materials having a vapour pressure greater than about 0.001 Torr (>0.00013 kPa) at 25° C., may smell citrusy, green, aquatic light and fresh, and tend to be noticeable for only a few minutes after being applied to a substrate. Other examples of highly volatile PRMs, such as floral, aromatic or fruity notes, may be noticeable for several hours after application to the substrate. Even so, it is still desirable to have the highly volatile PRMs remaining on the applied substrate for long periods of time after application (e.g., greater than 3 hrs, 4 hrs, 5 hrs, 6 hrs, 8 hrs or more all the way up to 24 hours).

Typically, the perceived intensity of the fragrance profile, particularly those aromas attributable to the highly volatile PRMs, are initially dominant but decreases rapidly over time due to their quick evaporation. This is a problem because some consumers desire prolonged intensity of select aromas, particularly the floral, fruity or aromatic aromas derived from the highly volatile PRMs. Simply adding higher levels of highly volatile PRMs creates an initial impression of a harsh and unfinished fragrance that consumers do not find acceptable. Additionally this does not provide any significant fragrance longevity due to their fast evaporation. This approach of using higher levels of materials therefore comes at a significant cost with no improvement in performance over time. Other previous attempts to overcome the problem have been through the use of high levels of low volatile PRMs. The unfortunate consequence of using high levels of low volatile PRMs is that they may impart particularly undesirable aroma characters, such as for example, musky, woody, ambery, warm and sweet, which can overpower and dominate the more desirable fragrance characters over time, particularly over longer periods of time. Thus, the unique challenge remains of selectively extending the more desirable aromas attributable from the highly volatile PRMs, and preferably, extending these desirable aromas over long periods of time.

Recently, ionic liquids have been used in the fragrance industry for dealing with solvent applications of the synthesis of fragrance materials or with the extractions of naturally derived PRMs (Sullivan, N., Innovations in Pharma. Tech. 2006, 20:75-77). For example, Forsyth et al. investigated the utilization of ionic liquid solvents for the synthesis of lily-of-the-valley fragrance material and fragrance intermediate Lilial (Forsyth et al., J. Mol. Cat. A. 2005, 231:61-66). Additionally, the utilization of ionic liquids to suppress evaporation of all types of fragrance materials in consumer products has also been gaining attention (Davey P., Perfumer Flavorist 2008, 33(4):34-35). For instance, ionic liquids have been used as “fixatives” with fragrance compositions to delay the rate of evaporation of the entire perfume component to impart increased stability/longevity of all types of fragrance materials in a composition (Petrat et al., US2006/0166856). Ionic liquids have also been used as pro-fragrances where PRM is appended covalently to either the cation or the anion (Rogers et al., US2012/046244; Blesic et al., RSC Advances, 2013, 3:329-333).

Accordingly, as discussed above, where these attempts have mentioned the use of ionic liquids as fixatives, they have focused only on the use of the ionic liquids for delaying the evaporation of all types of PRMs in the composition. As such, these teachings still have limitations, and do not adequately teach how to use ionic liquids in fragrance compositions for delaying evaporation of select PRMs, preferably highly volatile PRMs. Therefore, there remains a need for a fragrance composition that comprises ionic liquids to control in a targeted manner, decreases in the evaporation and/or release of PRMs, preferably highly volatile PRMs, from the fragrance composition. There is also a need for a fragrance composition that has a substantial proportion of the PRMs, preferably the highly volatile PRMs, remaining on the applied substrate for even long periods of time after application (e.g., greater than 3 hrs, 4 hrs, 5 hrs, 6 hrs, 8 hrs or more all the way up to 24 hours).

### SUMMARY OF THE INVENTION

In a first aspect, the present invention is directed to a fragrance composition comprising (a) from 0.001% to 99.9% by weight of the total fragrance composition of a perfume raw material, wherein the perfume raw material displays a negative deviation from Raoult's Law; and (b) from 0.01% to 99% by weight of the total fragrance composition of at least one ionic liquid comprising: (i) an anion; and (ii) a cation; wherein the ionic liquid is a liquid at temperatures lower than 100° C., preferably at ambient temperature. Preferably, the perfume raw material displays a negative deviation from Raoult's Law as determined by the D2879:2010 Standard Test Method (“ASTM D2879 Isoteniscope Method”) or by the Gas-Phase Infrared Spectroscopy Method as described herein.

In another aspect of the present invention, a fragrance composition comprising an ionic liquid as provided above and at least one highly volatile perfume raw material having a vapour pressure greater than 0.001 Torr (>0.00013 kPa) at 25° C. and the highly volatile perfume raw material is present in an amount from 0.001 wt % to 99.9 wt %, preferably from 0.01 wt % to 99 wt %, relative to the total weight of the perfume raw materials. Of this aspect, wherein the perfume raw material comprises at least 2, 3, 4, 5, 6 or more highly volatile perfume raw materials.

In still another aspect of the present invention, use of fragrance compositions according to the present invention in various products, preferably for personal care applications, and to the preparation thereof. In yet still another aspect of the present invention, a method for treating a targeted substrate using the fragrance composition is provided. These, and other features of the present invention, will become apparent to one skilled in the art upon review of the following detailed description when taken in conjunction with the appended claims.

### BRIEF DESCRIPTION OF THE DRAWINGS

While the specification concludes with claims particularly pointing out and distinctly claiming the invention, it is believed that the invention will be better understood from the following description of the accompanying figures wherein:

FIG. 1 provides a Gas-Phase Infrared (“IR”) spectrum for dimethyl benzyl carbinyl butyrate (“DMBCB”) at 25° C., with a path length of 8 metres and an analytical region between 4,000 and 1,000 cm⁻¹ according to the Gas-Phase Infrared Spectroscopy Method.

FIG. 2 provides a Gas-Phase IR spectrum for Citrowanil® B at 40° C., with a path length of 8 metres and an analytical region between 4,000 and 1,000 cm⁻¹ according to the Gas-Phase Infrared Spectroscopy Method.

FIG. 3 provides a Gas-Phase IR spectrum for an evacuated cell with an analytical region between 4,000 and 1,000 cm⁻¹ according to the Gas-Phase Infrared Spectroscopy Method.

FIG. 4 provides ¹H NMR spectrum of 1-butyl-3-methylimidazolium prolinate (CDCl₃, 500 MHz) from Example 2.

FIG. 5 provides ¹³C NMR spectrum of 1-butyl-3-methylimidazolium prolinate (CDCl₃, 125 MHz) from Example 2.

FIG. 6a) provides plots of absorbance of DMBCB in the gas phase at 25° C. for DMBCB dissolved in Ionic Liquid 8 from Example 3a.

FIG. 6b) provides plots of absorbance of DMBCB in the gas phase at 25° C. for DMBCB dissolved in Ionic Liquid 9 from Example 3a.

FIG. 7 provides a graph for an ideal solution that follows “Raoult's Law” such that the total vapour pressure and the partial vapour pressures are proportional to the mole fractions of the components.

### DETAILED DESCRIPTION OF THE INVENTION

### Definitions

As used herein, articles such as “a” and “an” when used in a claim, are understood to mean one or more of what is claimed or described.

As used herein, the terms “include”, “includes” and “including” are meant to be non-limiting.

As used herein, the term “Citrowanil® B” refers to the PRM having the chemical name benzenepropanenitrile, α-ethenyl-α-methyl- and structure:

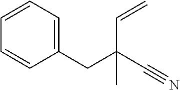

As used herein, the term “dimethyl benzyl carbinyl butyrate” (“DMBCB”) refers to the PRM having the chemical name 2-methyl-1-phenylpropan-2-yl butanoate and structure:

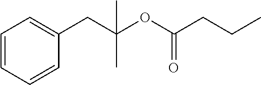 [SMILES: CCCC(=O)OC(C)(C)Cc1ccccc1]

As used herein, the term “fragrance composition” includes a stand alone product such as, for example, a fine fragrance composition intended for application to a body surface, such as for example, skin or hair, i.e., to impart a pleasant odor thereto, or cover a malodour thereof. The fine fragrance compositions are generally in the form of perfume concentrates, perfumes, eau de parfums, eau de toilettes, aftershaves, colognes, body splashes, or body sprays. The fine fragrance compositions may be ethanol based compositions. The term “fragrance composition” may also include a composition that can be incorporated as part of another product such as, for example, a cosmetic composition which comprises a fragrance material for the purposes of delivering a pleasant smell to drive consumer acceptance of the cosmetic composition. Additional non-limiting examples of “fragrance composition” may also include facial or body powder, foundation, body/facial oil, mousse, creams (e.g., cold creams), waxes, sunscreens and blocks, deodorants, bath and shower gels, lip balms, self-tanning compositions, masks and patches.

As used herein, the term “fragrance profile” means the description of how the fragrance is perceived by the typical human nose after it has been applied to a substrate. It is a result of the combination of the PRMs, if present, of a fragrance composition. A fragrance profile is composed of 2 characteristics: ‘intensity’ and ‘character’. The ‘intensity’ relates to the perceived strength whilst ‘character’ refers to the odor impression or quality of the perfume, i.e., fruity, floral, woody, etc.

As used herein, the terms “perfume” refers to the component in the fragrance composition that is formed of perfume raw materials, i.e., ingredients capable of imparting or modifying the odor of skin or hair or other substrate.

As used herein, the terms “perfume raw material” (“PRM”), “perfume raw materials” (“PRMs”), and “fragrance materials” are used interchangeably and relate to a perfume raw material, or a mixture of perfume raw materials, that are used to impart an overall pleasant odor or fragrance profile to a fragrance composition. “Perfume raw materials” can encompass any suitable perfume raw materials for fragrance uses, including materials such as, for example, alcohols, aldehydes, ketones, esters, ethers, acetates, nitriles, terpene hydrocarbons, nitrogenous or sulfurous heterocyclic compounds and essential oils. However, naturally occurring plant and animal oils and exudates comprising complex mixtures of various chemical components are also know for use as “perfume raw materials”. The individual perfume raw materials which comprise a known natural oil can be found by reference to Journals commonly used by those skilled in the art such as “Perfume and Flavourist” or “Journal of Essential Oil Research”, or listed in reference texts such as the book by S. Arctander, Perfume and Flavor Chemicals, 1969, Montclair, N.J., USA and more recently re-published by Allured Publishing Corporation Illinois (1994). Additionally, some perfume raw materials are supplied by the fragrance houses (Firmenich, International Flavors & Fragrances, Givaudan, Symrise) as mixtures in the form of proprietary specialty accords. Non-limiting examples of the perfume raw materials useful herein include pro-fragrances such as acetal pro-fragrances, ketal pro-fragrances, ester pro-fragrances, hydrolyzable inorganic-organic pro-fragrances, and mixtures thereof. The perfume raw materials may be released from the pro-fragrances in a number of ways. For example, the fragrance may be released as a result of simple hydrolysis, or by a shift in an equilibrium reaction, or by a pH-change, or by enzymatic release or by thermal change or by photo-chemical release.

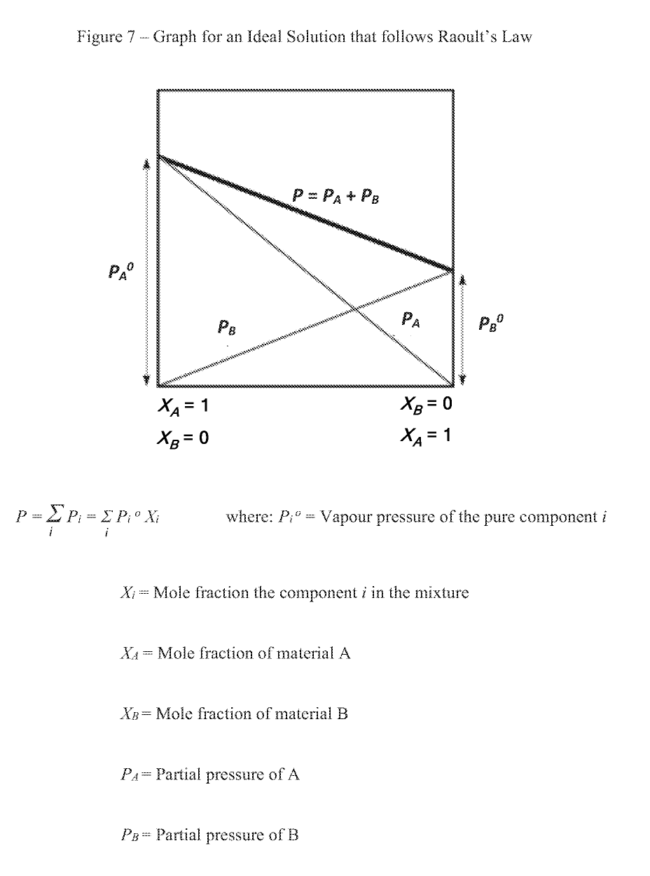

As used herein, the term “Raoult's Law” refers to the behaviour of the vapour pressure of the components of an ideal solution (Atkins, P. W. and Paula, J. D., Atkins' Physical Chemistry, 9^{th} Edit. (Oxford University Press Oxford, 2010). In an “ideal solution” the interaction between the different chemical species of the solution are the same as the self-interaction within the chemical species such that when the solution is formed the enthalpy of mixing is zero. The graph for an ideal solution in a 2-component system is shown in FIG. 7.

With continued reference to FIG. 7, the partial pressure of each component, Pᵢ, is equal to the pressure of the pure component, Pᵢ⁰, multiplied by its mole fraction, Xᵢ. Ideal mixtures, that therefore by definition obey Raoult's Law, are usually mixtures of nearly identical structures and properties.

When mixtures do not follow Raoult's Law, they are termed non-ideal solutions. The activity coefficient, γ, describes the degree of deviation from ideality. The activity coefficient for component i at a mole fraction on X is described as:

γ_{iX}=P_{iX}/(P_{iX})_{ideal}

γ_{iX}=P_{iX}/(Xᵢ Pᵢ⁰)

where P_{iX} is the measured partial vapour pressure over a solution of PRM i at mole fraction X and (P_{iX})_{ideal} is the calculated ideal partial vapour pressure based on the mole fraction Xᵢ and the measured vapour pressure of the pure component Pᵢ⁰.

Alternatively the activity coefficient, γ, can also be determined by the concentrations in the gas-phase wherein,

γ_{iX}=c_{iX}/(c_{iX})_{ideal}

γ_{iX}=c_{iX}/(Xᵢ cᵢ⁰)

where c_{iX} is the measured concentration over a solution of PRM i at mole fraction X and c_{iXideal} is the calculated ideal concentration based on the mole fraction Xᵢ and the measured concentration of the pure component cᵢ⁰.

In addition, when relative concentrations (rc) rather than absolute gas-phase concentrations are measured, as with Infrared Gas-Phase Spectroscopy, the absolute concentrations can be substituted for relative concentration into the equation above, so that γ_{iX}=rc_{iX}/(Xᵢ rcᵢ⁰)

For ideal solutions, γ=1. Non-ideality can result in two alternative vapour pressure behaviours: (i) negative deviation from Raoult's Law (i.e., γ<1), wherein the vapour pressure is lower than that predicted for ideal behaviour or (ii) positive deviation from Raoult's Law (i.e., γ>1) wherein the vapour pressure is higher than predicted for ideal behaviour.

The present invention is directed at ionic liquids that when formulated into a fragrance composition will give rise to a negative deviation from Raoult's Law for one or more of the PRMs for which the activity coefficient (γ) is less than 1 at one of the mole fractions between 0.05 and 0.8 of the PRM.

Without wishing to be bound by theory, a negative deviation from Raoult's Law may indicate similarities of polarity and/or structure between the PRMs and the ionic liquid reducing the PRMs' ability to escape the liquid phase and go into the headspace. When this happens, the vapour pressure of the resultant mixture will be lesser than expected from Raoult's Law and thus show a negative deviation from the ideal solution behaviour, wherein the activity coefficient (γ) is less than 1.

The negative deviation can be determined as follows:
1. Determine the pure PRM vapour pressure Pᵢ⁰ or the pure PRM relative gas-phase concentration, rcᵢ⁰.
2. Calculate Raoult's Law ideal PRM vapour pressure (P_{iX})_{ideal} or the ideal PRM relative gas-phase concentration (c_{iX})_{ideal} at different PRM mole fractions (e.g., Xᵢ=0.05, 0.2, 0.4, 0.6, or 0.8).
3. Measure the PRM vapour pressure P_{iX} or relative gas-phase concentration rc_{iX} at different mole fractions (e.g., Xᵢ=0.05, 0.2, 0.4, 0.6, or 0.8).
4. Determine the activity coefficient (γ) at different mole fractions (e.g., Xᵢ=0.05, 0.2, 0.4, 0.6, or 0.8) according to the equation above.
5. A PRM is deemed to have a negative deviation if any of the activity coefficients are less than 1 for any of the mole fractions (e.g., Xᵢ=0.05, 0.2, 0.4, 0.6 or 0.8) of the PRM.

Whereby the vapour pressures of a PRM can be measured by the ASTM D2879:2010 Standard Test Method (“ASTM D2879 Isoteniscope Method”) for Vapour Pressure-Temperature Relationship and Initial Decomposition Temperature of Liquids by Isoteniscope with the variations as described herein. Alternatively, vapour pressure could also be measured using the vapour pressure apparatus described in Husson et al., Fluid Phase Equilibria 294 (2010) pp.98-104. Without wishing to be bound by theory, since ionic liquids exhibit effectively zero vapour pressure at room temperature, the measured vapour pressure is the vapour pressure of the volatile components (i.e., PRMs) and therefore for systems with only one volatile component these approaches measure the vapour pressure of the PRM.

However, water may be present in either the ionic liquid or the PRM and hence can also contribute to the vapour pressure measured by the methods above. This issue can be mitigated by thoroughly drying both the ionic liquid and PRM using standard techniques known in the art as described in the methods section herein. In addition, a correction factor may be applied to the measured vapour pressure to remove the portion of the vapour pressure that is attributable to water present in the ionic liquid. This measurement is then taken as the vapour pressure of the pure ionic liquid, since this is the vapour pressure due to the presence of water in the ionic liquid, proportional to the molar fraction of ionic liquid in the sample under consideration, as explained in the methods section.

Preferably, an alternative method that can determine the relative gas-phase concentrations of particular components involves the use of infrared (“IR”) spectroscopy. In particular, the infrared spectroscopy of the gas-phase is such a method that will distinguish between the chemicals in a simple multi-component system, in this case water and PRM. Molecules absorb specific frequencies of the electromagnetic spectrum that are characteristic of their structures. This technique is typically used to study organic compounds using radiation in the mid-IR range of 4,000-400 cm⁻¹. This provides a well defined fingerprint for a given molecule where IR light absorbance (or transmittance) is plotted on the vertical axis vs. frequency or wavelength on the horizontal axis, in units of reciprocal centimeters (cm⁻¹) or wavenumbers. Additionally to the materials contained in the enclosed headspace of the cell, atmospheric carbon dioxide is detected by the IR beam externally to the cell.

A gas-phase IR cell with heating jacket enables us to create a closed headspace at equilibrium at a specific temperature. The IR spectrometer scans the headspace and provides the fingerprint of the gaseous mixture. Specific peaks at particular wavenumbers in the spectra can be identified as typical of the components, as described in the method. The absorbance at a particular wavenumber is proportional to the gas-phase concentration, and hence vapour pressure, of the specific component identified at that wavenumber. The relative concentration is obtained by normalizing the absorbance at a particular wavenumber for a given sample versus the absorbance at that same wavenumber for the pure PRM.

If quantification is desirable, then it can be achieved by adding a known very small quantity of the volatile material (e.g., PRM), to the gas cell and taking the spectra at a temperature where all the volatile material is in the gas phase. This will then enable conversion between relative and absolute gas-phase concentrations. However, for the purposes of calculating the activity coefficient, as described above, this is not necessary as the activity coefficient is itself a ratio of concentrations.

As used herein, and unless defined otherwise, the term “vapour pressure” or “VP” means the pressure in a vacuum of the vapour in equilibrium with its condensed phase at a defined temperature for a given chemical species. It defines a chemical species' propensity to be in the gas phase rather than the liquid or solid state. The higher the vapour pressure, the greater the proportion of the material that will, at equilibrium, be found in a closed headspace. It is also related to the rate of evaporation of a perfume raw material which is defined in an open environment where material is leaving the system. Unless defined otherwise, the pure vapour pressure of a single material is calculated according to the reference program Advanced Chemistry Development (ACD/Labs) Software Version 2015 (or preferably the latest version update).

As used herein, and unless defined otherwise, the term “relative gas-phase concentration” means the relative concentration a vacuum of the vapour in equilibrium with its condensed phase at a defined temperature for a given chemical species. It defines a chemical species' propensity to be in the gas phase rather than the liquid or solid state. The higher the relative gas-phase concentration, the greater the proportion of the material that will, at equilibrium, be found in the gas-phase in a closed headspace. It is also related to the rate of evaporation of a perfume raw material in an open environment where material is leaving the system.

Certain chemical functional groups named here are preceded by a shorthand notation indicating the total number of carbon atoms that are to be found in the indicated chemical group. For example: C₁-C₂₀ alkyl describes an alkyl group having a total of 1 to 20 carbon atoms (e.g. C₁₀ implies C₁₀H₂₁). The total number of carbons in the shorthand notation does not include carbons that may exist in substituents of the group described. Unless specified to the contrary, the following terms have the following meaning:

“Amino” refers to the —NH₂ functional group.

“Cyano” refers to the —CN functional group.

“Halo” refers to fluoro, chloro, bromo, or iodo.

“Halide” refers to a halide atom bearing a negative charge such as for example, fluoride (F⁻), chloride (Cl⁻), bromide (Br⁻), or iodide (I⁻).

“Hydroxyl” refers to the —OH functional group.

“Oxo” refers to the ═O substituent.

“Alkyl” refers to a group containing a straight or branched hydrocarbon chain consisting solely of carbon and hydrogen atoms, containing no unsaturation, having from 1 to 20 carbon atoms, preferably 1 to 12 carbon atoms, preferably 1 to 8, or preferably 1 to 6 carbon atoms, and which is attached to the rest of the molecule by a single bond, e.g., methyl, ethyl, propyl, 1-methylethyl (iso-propyl), butyl, pentyl, and the like. An alkyl may be optionally substituted.

“Alkenyl” refers to a group containing straight or branched hydrocarbon chain consisting solely of carbon and hydrogen atoms, containing at least one carbon-carbon double bond, having from 2 to 20 carbon atoms, preferably 2 to 12 carbon atoms, or preferably 1 to 8 carbon atoms, e.g., ethenyl, prop-2-enyl, but-1-enyl, pent-1-enyl, penta-1,4-dienyl, and the like. An alkenyl may be optionally substituted.

“Alkynyl” refers to a group containing straight or branched hydrocarbon chain consisting solely of carbon and hydrogen atoms, containing at least one carbon-carbon triple bond, having from 2 to 20 carbon atoms, preferably 2 to 12 carbon atoms, or preferably 1 to 8 carbon atoms, e.g., ethynyl, propynyl, butynyl, pentynyl, hexynyl, and the like. An alkynyl may be optionally substituted.

“Alkylene” or “alkylene chain” refers to a group containing straight or branched hydrocarbon chain linking the rest of the molecule to a group, consisting solely of carbon and hydrogen, containing no unsaturation and having from 1 to 12 carbon atoms, e.g., methylene, ethylene, propylene, butylene, and the like. An alkylene may be optionally substituted.

“Alkenylene” or alkenylene chain” refers to a straight or branched hydrocarbon chain linking the rest of the molecule to a group, consisting solely of carbon and hydrogen, containing at least one carbon-carbon double bond and having from 2 to 20 carbon atoms, preferably 2 to 12 carbon atoms, e.g., ethenylene, propenylene, butenylene, and the like. An alkenylene may be optionally substituted.

“Alkynylene” or “alkynylene chain” refers to a straight or branched hydrocarbon chain linking the rest of the molecule to a group, consisting solely of carbon and hydrogen, containing at least one carbon-carbon triple bond and having from 2 to 20 carbon atoms, e.g., propynylene, butynylene, and the like. An alkynylene may be optionally substituted.

“Alkoxy” refers to a functional group of the formula —OR, where Rₐ is an alkyl chain as defined above containing 1 to 20 carbon atoms, preferably 1 to 12 carbon atoms. An alkoxy may be optionally substituted.

“Alkoxyalkyl” refers to a functional group of the formula —Rₐ₁—O—Rₐ₂ where Rₐ₁ is an alkylene as defined above and Rₐ₂ is an alkyl chain as defined above containing 1 to 20 carbon atoms, preferably 1 to 12 carbon atoms. An alkoxyalkyl may be optionally substituted.

“Aryl” refers to aromatic monocyclic or multicyclic hydrocarbon ring system consisting only of hydrogen and carbon, and preferably containing from 6 to 18 carbon atoms, preferably 6 to 10 carbon atoms, where the ring system is aromatic (by the Hückel definition). Aryl groups include but are not limited to groups such as phenyl, naphthyl, anthracenyl. The term “aryl” or the prefix “ar” (such as in “aralkyl”) is meant to include aryls that may be optionally substituted.

“Arylene” refers to a linking aryl group, and where the aryl is as defined above.

“Cycloalkyl” refers to a stable saturated mono-cyclic or polycyclic hydrocarbon group consisting solely of carbon and hydrogen atoms, which may include fused or bridged ring systems, having from 3 to 15 carbon atoms, preferably having from 3 to 10 carbon atoms or preferably from 3 to 7 carbon atoms. A cycloalkyl may be optionally substituted.

“Cycloalkylalkyl” refers to a functional group of the formula —RₐR_{d}, where Rₐ is an alkylene as defined above and R_{d} is a cycloalkyl as defined above.

“Haloalkyl” refers to an alkyl as defined above that is substituted by one or more halogen groups, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. A haloalkyl may be optionally substituted.

“Heterocyclyl” refers to a stable 3- to 24-membered saturated ring which consists of 2 to 20 carbon atoms and from 1 to 6 heteroatoms selected from atoms consisting of nitrogen, oxygen, or sulfur. Unless stated otherwise specifically in the specification, the heterocyclyl may be a monocyclic, bicyclic, tricyclic or tetracyclic ring system, which may include fused or bridged ring systems; and the nitrogen, carbon or sulfur atoms in the heterocyclyl may be optionally oxidized; the nitrogen atom may be optionally quaternised. A heterocyclyl may be optionally substituted.

“Heterocyclylalkyl” refers to a functional group of the formula —RₐRₑ where Rₐ is an alkylene as defined above and Rₑ is a heterocyclyl as defined above, and if the heterocyclyl is a nitrogen-containing heterocyclyl, the heterocyclyl may be attached to the alkylene at the nitrogen atom. A heterocyclylalkyl may be optionally substituted.

“Heteroaryl” refers to a 5- to 20-membered aromatic ring which consists of 1 to 17 carbon atoms and from 1 to 3 heteroatoms selected from atoms consisting of nitrogen, oxygen and sulfur. The heteroaryl may be a monocyclic, bicyclic, tricyclic or tetracyclic ring system, which may include fused or bridged ring systems. A heteroaryl may be optionally substituted.

“Heteroarylalkyl” refers to a functional group of the formula —RₐR_{f} where Rₐ is an alkylene as defined above and R_{f} is a heteroaryl as defined above. A heteroarylalkyl may be optionally substituted.

“Optionally substituted” means that the subsequently described event of circumstances may or may not occur and that the description includes instances where the event or circumstance occurs and instances in which it does not. For example, unless specified otherwise, “optionally substituted” means that the chemical moiety may or may not be substituted by one or more of the following groups: alkyl, alkenyl, halo, haloalkenyl, cyano, nitro, aryl, cycloalkyl, heterocyclyl, heteroaryl, oxo, —OR^{10b}, —OC(O)—R^{10b}, —N(R^{10b})₂, —C(O)R^{10b}, —C(O)OR^{10b}, —C(O)N(R^{10b})₂, —N(R^{10b})C(O)OR^{12b}, —N(R^{10b})C(O)R^{12b}, —N(R^{10b})S(O)ₜR^{12b} (where t is 1 to 2), —S(O)ₜOR^{12b} (where t is 1 to 2), —S(O)ₓR^{12b} (where x is 0 to 2) and —S(O)ₜN(R^{10b})₂ (where t is 1 to 2) where each R^{10b} is independently hydrogen, alkyl, haloalkyl, cycloalkyl, cycloalkylalkyl, aryl (optionally substituted with one or more halogen groups), aralkyl, heterocyclyl, heterocyclylalkyl, heteroaryl or heteroarylalkyl; and each R^{12b} is alkyl, haloalkyl, cycloalkyl, cycloalkylalkyl, aryl, aralkyl, heterocyclyl, heterocyclylalkyl, heteroaryl or heteroarylalkyl, and where each of the above substituents is unsubstituted unless otherwise indicated.

It is understood that the test methods that are disclosed in the Test Methods section of the present application must be used to determine the respective values of the parameters of the present invention as described and claimed herein.

In all embodiments of the present invention, all percentages are by weight of the total fragrance composition, as evident by the context, unless specifically stated otherwise. All ratios are weight ratios, unless specifically stated otherwise, and all measurements are made at 25° C., unless otherwise designated.

### Ionic Liquids

Surprisingly, it has been found that ionic liquids can be used to alter the display of PRMs from a fragrance composition. In particular, the applicants have discovered that fragrance compositions comprising ionic liquids will have delayed evaporation of some of the PRMs, preferably the highly volatile PRMs, from a surface in an open system. As a result, less of the PRMs, preferably the highly volatile PRMs, are present in the air directly above the application site shortly after application to a substrate. A consequence of this delayed evaporation is that PRMs, preferably the highly volatile PRMs, applied to a substrate will be exhausted after a longer period of time (i.e., greater than 3 hrs, 4 hrs, 5 hrs, 6 hrs, 8 hrs or more all the way up to 24 hrs), as compared to the same fragrance composition absent of the ionic liquids. This may be observed as some PRMs, preferably the highly volatile PRMs, being perceived as olfactively stronger at later time points (e.g., greater than 3 hrs, 4 hrs, 5 hrs, 6 hrs, 8 hrs or more all the way up to 24 hrs after application) or more long-lasting. In particular, ionic liquids, according to the present invention, appear to aid in targeted delays in the evaporation, preferably the highly volatile PRMs from the fragrance composition.

Preferably, the ionic liquids useful in the present invention exhibit no measurable vapour pressure between 25° C. and 100° C. Thus, it is understood that the ionic liquids themselves make no measurable contribution to the vapour pressure of any mixture in which they are incorporated.

By incorporating the ionic liquids, it is desired that the partial vapour pressure of the individual PRMs, preferably the components derived from the highly volatile perfume raw materials, of the fragrance composition is decreased as measured by the ASTM D2879 Isoteniscope Method or the vapour pressure apparatus in Husson et al., Fluid Phase Equilibria, 294 (2010) pp. 98-104 in a closed system or preferably by the Infrared Gas-Phase Spectroscopy Method. The partial vapour pressure in a closed system is an approximation for the partial vapour pressure close to the application site. While not wishing to be bound by theory, it is believed that the initially reduced partial vapour pressure of the PRMs by the ionic liquids is caused by the attraction between the polar functionalities of the PRMs and the ionic liquids. Since PRMs are neutral molecules, the dominant mechanism for association between PRMs and ionic liquids will be via hydrogen bond formation. In order to induce a negative deviation from Raoult's Law, the hydrogen bonding between the PRM and the ionic liquid should be maximized. If attraction between a PRM and an ionic liquid is desired, and the PRM contains an alcohol or phenol functional group (i.e., the PRM contains both hydrogen-bond donor and acceptor sites), then the structure of the ionic liquid should be designed to have certain properties. For example, the ionic liquid should contain hydrogen-bond acceptor sites, or more preferably contain both hydrogen-bond acceptor sites, and hydrogen-bond donor sites. If the PRM contains ether, ketone, aldehyde or ester functional groups (i.e., the PRM contains only hydrogen-bond acceptor site(s), but no hydrogen-bond donor sites), then the ionic liquid should be designed to contain hydrogen-bond donor site(s). There must be a net attractive interaction between the ionic liquid and the PRM; hence weak repulsion interactions can be tolerated as long as the sum of all the attractive interactions is greater than the sum of all the repulsion interactions.

Thus, the ionic liquids can be designed to attract PRMs, preferably the highly volatile PRMs, and hence induce changes in the PRMs' vapour pressures as compared to the vapour pressures of an ideal mixture. It is desirable that ionic liquids when incorporated into fragrance compositions of the present invention will result in negative deviations from Raoult's Law, so that the ionic liquids attract the PRMs to delay their release into the surrounding headspace.

In an embodiment, the PRMs, preferably the highly volatile PRMs, in the fragrance composition comprising the ionic liquids according to the present invention display a negative deviation from Raoult's Law, wherein the activity coefficient (“γ”) is less than 1. In other embodiments, the fragrance composition of the present invention will give rise to a negative deviation from Raoult's Law for one or more of the PRMs for which the activity coefficient (γ) <1.0 or 0.95 or 0.90 or 0.85 or 0.80 or 0.75 or 0.70 or 0.65 or 0.60 or 0.55 or 0.50 or 0.45 or 0.40 or 0.35 or 0.30 or 0.25 or 0.20 or 0.15 or 0.10 or 0.05 at a mole fraction between 0.05 to 0.8 of the PRM.

Preferably, the perfume raw material displays the negative deviation from Raoult's Law having an activity coefficient (γ) less than 1 at a mole fraction between 0.05 and 0.8 of the perfume raw material, preferably at the mole fraction between 0.05 and 0.2, or preferably at the mole fraction between 0.2 and 0.4, or preferably at the mole fraction between 0.4 and 0.6, or preferably at the mole fraction between 0.6 and 0.8 of the perfume raw material.

Preferably, the perfume raw material displays the negative deviation from Raoult's Law having an activity coefficient (γ) less than 1 is determined by the D2879:2010 Standard Test Method (“ASTM D2879 Isoteniscope Method”), and the perfume raw material is present at mole fraction between 0.2 and 0.8 of the perfume raw material.

Preferably, the perfume raw material displays the negative deviation from Raoult's Law having an activity coefficient (γ) less than 1 is determined by the Gas-Phase Infrared Spectroscopy Method, and the perfume raw material is present at mole fraction between 0.05 and 0.8 of the perfume raw material.

As used herein, the term “ionic liquid” refers to a liquid which consists exclusively of ions and is present in a liquid form at temperatures lower than 100° C., preferably at ambient or room temperature (i.e., from 15° C. to 30° C.). Particularly preferred ionic liquids are suitable for use in fragranced consumer products and have to be choosen so as to exclude an adverse effect in terms of health or ecology on people, nature and the environment. For example, fragrance compositions, such as for example, perfumes, which may come into direct contact with humans preferably have minimal toxic effect. For other selected applications such as deodorants, however, it may be useful if in the fragrance composition, in particular the ionic liquids, there are microbiocidal properties for killing the microorganisms for suppressing malodours.

Ionic liquids have no effective vapour pressure (essentially zero) and may be easy to handle. Their polarity can be readily adjusted so as to be suitable to a wide range of PRMs. Furthermore, ionic liquids are odorless and will not impart an odor of their own when added into the fragrance compositions of the present invention. Particularly preferable ionic liquids are ones where the PRMs are fully miscible to form a single phase liquid. However, if the PRMs are not entirely miscible, or are immiscible, then co-solvents (e.g., triethyl citrate, or others as listed herein below) can be added to aid in the solubility of the PRMs.

Typically, ionic liquids may have high viscosities (i.e., greater than about 1,000 mPa·s) at room temperature. High viscosities can be problematic in formulating the fragrance compositions of the present invention. Therefore, in an embodiment, the present invention is preferably directed to ionic liquids (undiluted with adjuncts, co-solvents or free water) which have viscosities of less than about 1000 mPa·s, preferably less than about 750 mPa·s, preferably less than about 500 mPa·s, as measured at 20° C. In some embodiments, the viscosity of the undiluted ionic liquids are in the range from about 1 mPa·s to about 400 mPa·s, preferably from 1 mPa·s to about 300 mPa·s, and more preferably from about 1 mPa·s to about 250 mPa·s.

The viscosities of the ionic liquids and fragrance compositions containing therein can be measured on a Brookfield viscometer model number LVDVII+ at 20° C., with Spindle S31 at the appropriate speed to measure materials of differing viscosities. Typically, the measurement is performed at speed from 12 rpm to 60 rpm. The undiluted state is prepared by storing the ionic liquids in a desiccator containing a desiccant (e.g. anhydrous calcium chloride) at room temperature for at least 48 hrs prior to the viscosity measurement. This equilibration period unifies the amount of innate water in the undiluted samples.

It should be understood that the terms “ionic liquid”, “ionic liquids” and “ILs” refer to ionic liquids, ionic liquid composites and mixtures (or cocktails) of ionic liquids. For example, an ionic liquid may be formed from a homogeneous combination comprising one species of anion and one species of cation, or it can be composed of more than one species of cation and/or anion. Thus, an ionic liquid may be composed of more than one species of cation and one species of anion. An ionic liquid may further be composed of one species of cation and more than one species of anion. Finally, an ionic liquid may further be composed of more than one species of cation and more than one species of anion.

In another embodiment of the present invention, the ionic liquids may be selectively made to be hydrophobic by careful selection of the anions.

In yet another embodiment of the present invention, the ionic liquids (i.e., cation and anion) are essentially free of any of the following chemical elements: antimony, barium, beryllium, bromine, cobalt, chromium, fluorine, iodine, lead, nickel, selenium, or thallium. By “essentially free” it is meant that no cation or anion containing any of the foregoing chemical elements are intentionally added to form the ionic liquids of the present invention.

Preferably, the ionic liquids are essentially free of chemical materials that are prohibited for use in cosmetic products in various countries, such as for example, the European Commission, Health and Consumers, Cosmetics Regulation Annex II—“List of Substances Prohibited in Cosmetics Products” (http://ec.europa.eu/consumers/cosmetics/cosing/index.cfm?fuseaction=search.results&annex_v2=II&search), and the United States Food and Drug Administration List of “Prohibited & Restricted Ingredients” for cosmetic applications (http://www.fda.gov/cosmetics/guidanceregulation/lawsregulations/ucm127406.htm). The fragrance composition preferably has at least one ionic liquid with an anion according to the following structures.

The fragrance composition preferably has at least one ionic liquid with an anion independently selected from a compound of formulae (I), (II), (III), (IV), (V), (VI), (VII) or (VIII):

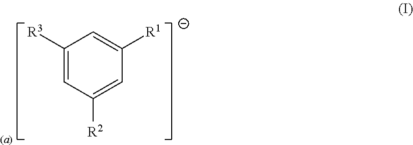

wherein:

R¹ and R³ are independently selected from hydrogen, cyano, hydroxy, C₁-C₂₀alkyl, C₁-C₂₀alkoxy or C₁-C₂₀alkoxyC₁-C₂₀alkyl;

R² is —R⁴—C(O)O, —R⁴—C(R⁵)CO, —R⁴—C(R⁵)C(O)O, C₁-C₂₀alkyl, C₂-C₂₀alkenyl, C₂-C₂₀alkynyl, C₁-C₂₀alkxoy, C₁-C₂₀alkoxyC₁-C₂₀alkyl, C₃-C₇cycloalkyl, C₃-C₇cycloalkylC₁-C₄alkyl, C₂-C₂₀heterocyclyl, optionally substituted C₆-C₁₀aryl, C₆-C₁₀arylC₁-C₁₀alkyl, C₁-C₁₀heteroaryl;

R⁴ is C₁-C₆alkylene, C₂-C₆alkeneylene, C₂-C₆alkynylene or a direct bond;

R⁵ is hydrogen, hydroxy, —NH or —N(R^{5a})₂; and

each R^{5a} is independently hydrogen or C₁-C₂₀alkyl;

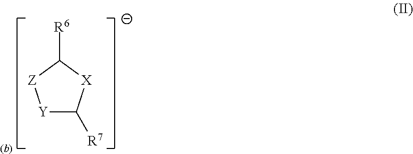

wherein:

X, Y and Z are independently selected from —CH₂—, —NH—, —S—, or —O—;

R⁶ is hydrogen, cyano, hydroxy, C₁-C₂₀alkyl, C₁-C₂₀alkoxy or C₁-C₂₀alkoxyC₁-C₂₀alkyl;

R^{6a} is C₁-C₆alkylene, C₂-C₆alkeneylene, C₂-C₆alkynylene or a direct bond;

R^{6b} is hydrogen, hydroxy, —NH or —N(R^{6c})₂;

each R^{6c} is independently hydrogen or C₁-C₂₀alkyl, and

R⁷ is —C(O)O, —R^{6a}—C(R^{6b})CO, —R^{6a}—C(R^{6b})C(O)O, C₁-C₂₀alkyl, C₂-C₂₀alkenyl, C₂-C₂₀alkynyl, C₁-C₂₀alkxoy, C₁-C₂₀alkoxyC₁-C₂₀alkyl, C₃-C₇cycloalkyl, C₃-C₇cycloalkylC₁-C₄alkyl, C₂-C₂₀heterocyclyl, optionally substituted C₆-C₁₀aryl, C₆-C₁₀arylC₁-C₁₀alkyl, C₁-C₁₀heteroaryl;

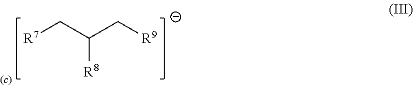

wherein:

R⁷ is —C(R¹⁰)N(R¹¹)₂, —C(O)O, or —S—R¹¹;

R⁸ is hydrogen or C₁-C₂₀alkyl;

R⁹ is —C(O)O or —C(O)N(R¹¹)₂;

R¹⁰ is hydroxy; and

each R¹¹ is independently hydrogen or C₁-C₂₀alkyl;

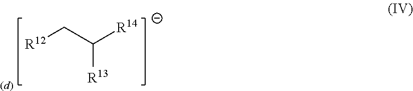

wherein:

R¹² is —C(R¹⁵)₃;

R¹³ is hydrogen or —N(R¹⁶)₂;

R¹⁴ is —R^{14a}—C(O)O;

R^{14a} is C₁-C₆alkylene, C₂-C₆alkeneylene, C₂-C₆alkynylene or a direct bond;

each R¹⁵ is independently selected from hydrogen, C₁-C₂₀alkyl or hydroxy; and

each R¹⁶ is independently selected from hydrogen or C₁-C₂₀alkyl;

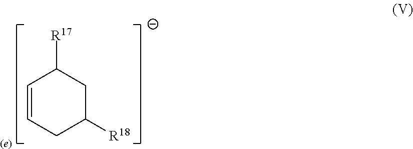

wherein:

R¹⁷ is hydrogen, cyano, hydroxy, —C(O), C₁-C₂₀alkyl, C₁-C₂₀alkoxy or C₁-C₂₀alkoxyC₁-C₂₀alkyl; and

R¹⁸ is —R^{18a}—C(O)O; —R^{18a}—C(R^{18b})CO, —R^{18a}—C(R^{18b})C(O)O, C₁-C₂₀alkyl, C₂-C₂₀alkenyl, C₂-C₂₀alkynyl, C₁-C₂₀alkxoy, C₁-C₂₀alkoxyC₁-C₂₀alkyl, C₃-C₇cycloalkyl, C₃-C₇cycloalkylC₁-C₄alkyl, C₂-C₂₀heterocyclyl, optionally substituted C₆-C₁₀aryl, C₆-C₁₀arylC₁-C₁₀alkyl, C₁-C₁₀heteroaryl;

R^{18a} is C₁-C₆alkylene, C₂-C₆alkeneylene, C₂-C₆alkynylene or a direct bond;

R^{18b} is hydrogen, hydroxy, —NH or —N(R^{18c})₂; and

each R^{18c} is independently hydrogen or C₁-C₂₀alkyl;

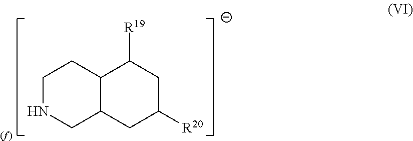

wherein:

R¹⁹ is hydrogen, cyano, hydroxyl, —C(O), C₁-C₂₀alkyl, C₁-C₂₀alkoxy or C₁-C₂₀alkoxyC₁-C₂₀alkyl; and

R²⁰ is —R^{20a}—C(O)O, —R^{20a}—C(R^{20b})CO, —R^{20a}—C(R^{20b})C(O)O, C₁-C₂₀alkyl, C₂-C₂₀alkenyl, C₂-C₂₀alkynyl, C₁-C₂₀alkxoy, C₁-C₂₀alkoxyC₁-C₂₀alkyl, C₃-C₇cycloalkyl, C₃-C₇cycloalkylC₁-C₄alkyl, C₂-C₂₀heterocyclyl, optionally substituted C₆-C₁₀aryl, C₆-C₁₀arylC₁-C₁₀alkyl, C₁-C₁₀heteroaryl;

R^{20a} is C₁-C₆alkylene, C₂-C₆alkeneylene, C₂-C₆alkynylene or a direct bond;

R^{20b} is hydrogen, hydroxy, —NH or —N(R^{20c})₂; and

each R^{20c} is independently hydrogen or C₁-C₂₀alkyl;

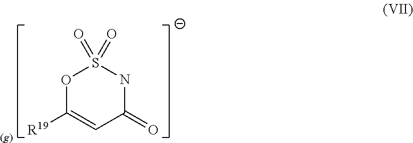

wherein:

R¹⁹ is hydrogen, cyano, alkyl, alkoxy, and alkoxyalkyl;

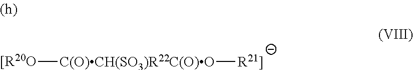

wherein:

R²⁰ and R²¹ are independently selected from the group consisting of alkyl or alkenyl, provided that the alkyl is not substituted with nitro, azido or halide; and

R²² is alkylene, heteroarylene, arylene, or cycloalkylene; and
(i) combinations thereof.

Preferably, the anion is independently selected from the group consisting of: 3,5-dihydroxybenzoic acid; 5 -hydroxytetrahydrofuran-3-carboxylate; 5-formylcyclohex-3-ene-1-carboxylate; 4-hydroxy-1,3-thiazolidine-2-carboxylate; 3′,5′-dihydroxybiphenyl-3-carboxylate; hydroxy(phenyl)acetate; 5-amino-5-hydroxypentanoate; 4-(3,4-dihydroxyphenyl)butanoate; 5-amino-3-methyl-5-oxopentanoate; 5-hydroxydecahydroisoquinoline-7-carboxylate; 2-amino-3-phenylpropanoate; 2-amino-3-(3-hydroxyphenyl)propanoate; 2-amino-4-hydroxy-4-methylpentanoate; 2-amino-4-hydroxy-4-methylhexanoate; 2-amino-4-(methylsulfanyl)butanoate; L-prolinate; 6 methyl-3,4-dihydro-1,2,3-oxathiazin-4-one 2,2-dioxide; 1,4-bis(2-ethylhexoxy)-1,4-dioxobutane-2-sulfonate; and combinations thereof.

The preparation of the anions is generally known and can take place, for example, as described in (P. Wasserscheid and T. Welton (Eds.), Ionic Liquids in Synthesis, 2^{nd} Edition, Wiley-VCH, 2008). In addition, the alkali metal salts of many anions are also available commercially.

The fragrance composition preferably has at least one ionic liquid with a cation according to the following structures.

Preferably, the cation is independently selected from the group consisting of:

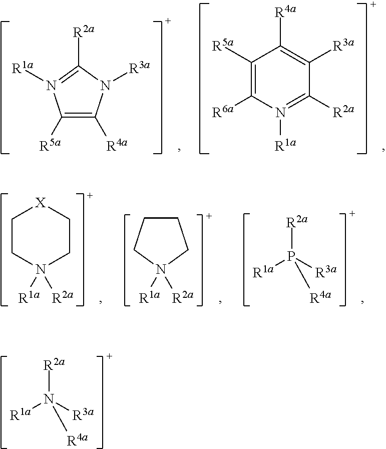

and combinations thereof;

wherein:
X is CH₂ or O;
each R^{1a}, R^{3a}, and R^{4a} are independently selected from hydrogen, C₁-C₂₀ alkyl, C₁-C₂₀ alkenyl, C₁-C₂₀ alkynyl, C₁-C₂₀ alkoxy, C₁-C₂₀ alkoxyC₁-C₂₀alkyl, C₃-C₇cycloalkyl, C₃-C₇cycloalkylC₁-C₄alkyl, C₂-C₂₀heterocyclyl, C₆-C₁₀aryl, C₆-C₁₀arylC₁-C₁₀alkyl, C₁-C₁₀heteroaryl, halo, haloC₁-C₂₀alkyl, hydroxyl, hydroxyC₁-C₂₀alkyl, or —N(R^{6a})₂;
each R^{2a} is independently selected from hydrogen, C₁-C₂₀ alkyl, C₁-C₂₀ alkenyl, or C₁-C₂₀ alkynyl;
each R^{5a} is independently selected from hydrogen, C₁-C₂₀ alkyl, C₁-C₂₀ alkenyl, C₁-C₂₀ alkynyl, —R^{7a}—OR^{8a}, or —R^{7a}—OR^{7a}—OR^{8a};
each R^{6a} is independently selected from hydrogen, alkyl, alkenyl, alkynyl, haloalkyl, alkoxyalkyl, cycloalkyl, cycloalkylalkyl, aryl, aralkyl, heterocyclyl, heterocyclyalkyl, heteroaryl, or heteroarylalkyl;
each R^{7a} is independently selected from a direct bond, alkylene chain, alkenylene chain, or alkynylene chain; and
each R^{8a} is independently selected from a hydrogen, alkyl, alkenyl or alkynyl.

Preferably, the cation is independently selected from the group consisting of 1-butyl-3-methylimidazolium; (N-ethyl-2-(2-methoxyethoxy)-N,N-dimethylethanaminium); 2-(2-ethoxyethoxy)-N-ethyl-N,N-dimethylethanaminium; N-benzyl-N,N-dimethyloctan-1-aminium; N-benzyl-N,N-dimethylnonan-1-aminium; 2-(2-methoxyethoxy)-N-[2-(2-methoxyethoxy)ethyl]-N,N-dimethylethan-1-aminium; 1-ethanaminium, N,N,N-tris[2-(2-methoxyethoxy)ethyl]; and combinations thereof.

The methods for preparing the cations of the present invention are provided in the Examples section. The preparations are not intended to limit the scope of the present invention. In addition, some cations may be available commercially.

It may be advantageous if the fragrance composition has an ionic liquid which has one or more of the abovementioned salts. It is understood that the ionic liquids can comprise either a single anionic species and a single cationic species or a plurality of different anionic and cationic species. By using different anionic species and/or different cationic species, the properties of the ionic liquids can be matched in an optimal way to the PRMs and/or other components of the fragrance composition. In an embodiment of the invention, the ionic liquids consist of more than one anionic species.

Ionic liquids are formed by combining simply salts of a cation and an anion (e.g. sodium salt of the anion and chloride salt of the cation). Different ionic liquids can be synthesized such that the interactions between the ionic liquids and the solute (i.e., perfume raw materials) are optimized, preferably to provide for a negative deviation from Raoult's Law. Ionic liquids lend themselves to preparation via combinatorial or high-throughput chemistry. Some methods for preparing the ionic liquids of the present invention are provided in the Examples section. The preparations are not intended to limit the scope of the present invention.

### Fragrance Compositions

Applicants have surprisingly found that ionic liquids can be added to fragrance compositions to selectively delay the evaporation of some PRMs, preferably the highly volatile perfume raw materials, from solution. Such delay is desirable, for example, to decrease the initial partial pressure and concentration of certain PRMs in the headspace. This will result in less overpowering perfume materials when they are applied to the surface and more noticeable perfume materials at later time points after that. It may also lengthen the time frame in which some PRMs, preferably the highly volatile perfume raw materials, continue to be detectable in the headspace after application of the fragrance compositions.

Specifically, in one aspect, the present invention provides for a fragrance composition comprising a perfume raw material present with a negative deviation from Raoult's Law in an amount of from about 0.001 wt % to about 99.9 wt %, preferably from about 0.01 wt % to about 90 wt %, preferably from about 0.1 wt % to about 80 wt %, preferably from about 0.2 wt % to about 70 wt %, preferably from about 0.3 wt % to about 60 wt %, preferably from about 0.4 wt % to about 50 wt %, preferably from about 0.5 wt % to about 40 wt %, preferably from about 1 wt % to about 30 wt %, relative to the total weight of the fragrance composition. Further, the perfume raw material comprises at least one highly volatile perfume raw material having a vapour pressure greater than 0.001 Torr (>0.00013 kPa) at 25° C.

In another aspect, applicants have surprisingly discovered that by adding ionic liquids in a fragrance composition, the fragrance profile, particularly the portion of the fragrance profile which is derived from the highly volatile PRMs can be improved. For example, by “improved” it is meant that initially a lower fraction of the highly volatile PRMs are in the headspace than could be achieved in the absence of ionic liquids. The highly volatile PRMs would then be olfactively more noticeable at later time points (i.e., stronger, and/or more dominant), preferably for long periods of time after application, leading to noteable differences such as, for example, a different concentration profile and new characters, as compared to controls (i.e., compositions containing the highly volatile fragrance materials and no ionic liquids).

Typically, it has been very difficult to formulate fragrance profiles with an accord made from PRMs having a wide range of volatility, but especially an accord characteristic of the highly volatile PRMs, whereby the fragrance profile derived from the highly volatile PRMs can be detected later after its application versus a control. The present invention will allow perfumers to formulate fragrance composition using PRMs having a wide range of volatility, particularly the highly volatile PRMs. They can now create new fragrance characters and address a re-occurring consumer issue that particular fragrance profiles, particularly fragrance compositions containing floral, citrus, green, aquatic, aromatic and fruity notes, tend to evaporate too fast.

Such a solution as presented herein provides enhanced longevity of the fragrance profile, particularly amongst those fragrance compositions formulated from highly volatile PRMs having a vapour pressure of greater than 0.001 Torr (>0.00013 kPa) at 25° C. This provides the perfumer options to formulate accords having new fragrance profiles.

### Volatile Solvents

In yet another aspect, additional suitable solvents may be present in the fragrance composition of the present invention. For example, for perfume applications in particular, ethanol may be present in any of the fragrance compositions of the present invention, and more specifically, it will form from about 10 wt % to about 80 wt %, or even from about 25 wt % to about 75 wt % of the fragrance composition, or combinations thereof, relative to the total weight of the fragrance composition. Any acceptable quality of ethanol (preferably high-quality), compatible and safe for the specific intended use of the fragrance composition such as, for example, topical applications of fine fragrance or cosmetic compositions, and is convenient for use in the fragrance compositions according to the present invention.

### Low Volatility Co-Solvents

The fragrance composition may comprise a low volatility co-solvent or a mixture of low volatility co-solvents. As used herein, the term “low volatility co-solvents” include solvents that have a vapour pressure of less than 0.3 Torr (<0.040 kPa) at 25° C. Preferably, the low volatility co-solvents do not contribute significantly to the odor profile of the fragrance compositions. For example, for perfume applications, a low volatility co-solvent or a mixture of low volatility co-solvents may be present in any of the fragrance compositions of the present invention, and more specifically, it may form from about 0.1 wt % to about 50 wt %, or even from about 1 wt % to about 40 wt % of the fragrance composition, or combinations thereof, relative to the total weight of the fragrance composition. Non-limiting examples of suitable low volatility co-solvents include benzyl benzoate, diethyl phthalate, isopropyl myristate, propylene glycol, triethyl citrate, and mixtures thereof.

### Water

In yet another aspect, water may be present in any of the fragrance compositions of the present invention, and more specifically, it shall not exceed about 50 wt %, preferably about 40 wt % or less, relative to the total weight of the composition. Alternatively, water may be present in an amount of less than 50 wt %, less than 40 wt %, less than 30 wt %, less than 20 wt % or less than 10 wt %, wherein the wt % is relative to the total weight of the fragrance composition. When the fragrance composition is a cosmetic composition, the level of water should not be so high that the product becomes cloudy or phase separates thus negatively impacting the product aesthetics. It is understood that the amount of water present in the fragrance composition may be from the water present in the ethanol used in the fragrance composition, as the case may be.

### Propellants

The fragrance compositions described herein may include a propellant. Some examples of propellants include compressed air, nitrogen, inert gases, carbon dioxide, and mixtures thereof. Propellants may also include gaseous hydrocarbons like propane, butane, isobutene, cyclopropane, and mixtures thereof. Halogenated hydrocarbons like 1,1-difluoroethane may also be used as propellants. Some non-limiting examples of propellants include 1,1,1,2,2-pentafluoroethane, 1,1,1,2-tetrafluoroethane, 1,1,1,2,3,3,3-heptafluoropropane, trans-1,3,3,3-tetrafluoroprop-1-ene, dimethyl ether, dichlorodifluoromethane (propellant 12), 1,1-dichloro-1,1,2,2-tetrafluoroethane (propellant 114), 1-chloro-1,1-difluoro-2,2-trifluoroethane (propellant 115), 1-chloro-1,1-difluoroethylene (propellant 142B), 1,1-difluoroethane (propellant 152A), monochlorodifluoromethane, and mixtures thereof. Some other propellants suitable for use include, but are not limited to, A-46 (a mixture of isobutane, butane and propane), A-31 (isobutane), A-17 (butane), A-108 (propane), AP70 (a mixture of propane, isobutane and n-butane), AP40 (a mixture of propane, isobutene and butane), AP30 (a mixture of propane, isobutane and butane), and 152A (1,1 diflouroethane). The propellant may have a concentration from about 15%, 25%, 30%, 32%, 34%, 35%, 36%, 38%, 40%, or 42% to about 70%, 65%, 60%, 54%, 52%, 50%, 48%, 46%, 44%, or 42% by weight of the total fill of materials stored within the container.

### Antiperspirant Active

The fragrance compositions described herein may be free of, substantially free of, or may include an antiperspirant active (i.e., any substance, mixture, or other material having antiperspirant activity). Examples of antiperspirant actives include astringent metallic salts, like the inorganic and organic salts of aluminum, zirconium and zinc, as well as mixtures thereof. Such antiperspirant actives include, for example, the aluminium and zirconium salts, such as aluminium halides, aluminium hydroxohalides, zirconyl oxohalides, zirconyl hydroxohalides, and mixtures thereof.

### Other Ingredients

In yet another aspect, the fragrance composition consists essentially of the recited ingredients but may contain small amounts (not more than about 10 wt %, preferably no more than 5 wt %, or preferably no more than 2 wt % thereof, relative to the total weight of the composition) of other ingredients that do not impact on the fragrance profile, particularly the evaporation rate and release of the fragrance materials. For example, a fragrance composition may comprise stabilising or anti-oxidant agents, UV filters or quenchers, or colouring agents, commonly used in perfumery. There are a number of other examples of additional ingredients that are suitable for inclusion in the present compositions, particularly in compositions for cosmetic use. These include, but are not limited to, alcohol denaturants such as denatonium benzoate; UV stabilisers such as benzophenone-2; antioxidants such as tocopheryl acetate; preservatives such as phenoxyethanol, benzyl alcohol, methyl paraben, and propyl paraben; dyes; pH adjusting agents such as lactic acid, citric acid, sodium citrate, succinic acid, phosphoric acid, sodium hydroxide, and sodium carbonate; deodorants and anti-microbials such as farnesol and zinc phenolsulphonate; humectants such as glycerine; oils; skin conditioning agents such as allantoin; cooling agents such as trimethyl isopropyl butanamide and menthol; hair conditioning ingredients such as panthenol, panthetine, pantotheine, panthenyl ethyl ether, and combinations thereof; silicones; solvents such as hexylene glycol; hair-hold polymers such as those described in PCT Publication WO94/08557 (Procter & Gamble); salts in general, such as potassium acetate and sodium chloride and mixtures thereof.

In yet another aspect, the fragrance compositions for use in the present invention may take any form suitable for use, more preferably for perfumery or cosmetic use. These include, but are not limited to, vapour sprays, aerosols, emulsions, lotions, liquids, creams, gels, sticks, ointments, pastes, mousses, powders, granular products, substrates, cosmetics (e.g. semi-solid or liquid makeup, including foundations) and the like. Preferably the fragrance compositions for use in the present invention take the form of a vapour spray. Fragrance compositions of the present invention can be further added as an ingredient to other compositions, preferably fine fragrance or cosmetic compositions, in which they are compatible. As such they can be used within solid composition or applied substrates etc.

Therefore, it goes without saying that the fragrance compositions of the present invention encompasses any composition comprising any of the ingredients cited herein, in any embodiment wherein each such ingredient is independently present in any appropriate amount as defined herein. Many such fragrance compositions, than what is specifically set out herein, can be encompassed.

### Article of Manufacture

The fragrance composition may be included in an article of manufacture comprising a spray dispenser. The spray dispenser may comprise a vessel for containing the fragrance composition to be dispensed. The spray dispenser may comprise an aerosolised fragrance composition (i.e. a fragrance composition comprising a propellant) within the vessel as well. Other non-limiting examples of spray dispensers include non-aerosol dispensers (e.g. vapour sprays), manually activated dispensers, pump-spray dispensers, or any other suitable spray dispenser available in the art.

### Methods of Using the Fragrance Compositions

The fragrance composition of the present invention according to any embodiments described herein is a useful perfuming composition, which can be advantangeously used as consumer products for personal care application intended to perfume any suitable substrate. As used herein, the term “substrate” means any surface to which the fragrance composition of the present invention may be applied to without causing any undue adverse effect. For example, this can include a wide range of surfaces including human or animal skin or hair. Preferred substrates include body surfaces such as, for example, hair and skin, most preferably skin.

The fragrance composition of the present invention may be used in a conventional manner for fragrancing a substrate. An effective amount of the fragrance composition, typically from about 1 μL to about 10,000 μL, preferably from about 10 μL to about 1,000 μL, more preferably from about 25 μL to about 500 μL, or most preferably from about 50 μL to about 100 μL, or combinations thereof, is applied to the suitable substrate. Alternatively, an effective amount of the fragrance composition of the present invention is from about 1 μL, 10 μL, 25 μL or 50 μL to about 100 μL, 500 μL, 1,000 μL or 10,000 μL. The fragrance composition may be applied by hand or applied utilizing a delivery apparatus such as, for example, vaporizer or atomizer. Preferably, the fragrance composition is allowed to dry after its application to the substrate. The scope of the present invention should be considered to cover one or more distinct applications of the fragrance composition

In one embodiment, present invention preferably relates to fragrance compositions in the form of product selected from the group consisting of a perfume, an eau de toilette, an eau de parfum, a cologne, a body splash, an aftershave lotion or a body spray. Therefore, according to this embodiment, the present invention provides a method of modifying or enhancing the odor properties of a body surface, preferably hair or skin, comprising contacting or treating the body surface with a fragrance composition of the present invention.

In another aspect, the present invention is directed to a method of delaying evaporation rate of the fragrance profile of a fragrance composition, preferably by decreasing the volatility of the PRMs, preferably the components derived from the highly volatile PRMs, present in the fragrance composition. The method comprises bringing into contact or mixing at least one ionic liquid as described hereinabove with at least one highly volatile fragrance material according to the fragrance composition of the present invention.

In one embodiment, the fragrance profile of the fragrance composition of the present invention is detectable by a consumer up to certain time points, such as for example, greater than 3 hrs, 4 hrs, 5 hrs, 6 hrs, 8 hrs or more all the way up to 24 hrs after application of the fragrance composition to a substrate as compared to controls.

### Fragrance Materials

In order that the fragrance compositions can be developed with the appropriate fragrance profile for the present invention, the PRMs have been classified by their vapour pressure. For the purpose of clarity, when the PRMs refer to a single individual compound, its vapour pressure should be determined. In the case that the PRMs are a natural oil, extract or absolute, which comprises a mixture of several compounds, the vapour pressure of the complete oil should be treated as a mixture of the individual perfume raw material components. The individual components and their level, in any given natural oil or extract, can be determined by direct injection of the oil into a GC-MS column for analysis as known by one skilled in the art. In the scenario that the PRMs are a proprietary specialty accord, so called ‘bases’, the vapour pressure should preferably be obtained from the supplier. However, it is understood by one skilled in the art that they can physically analyze the composition of a full fragrance oil available commercially to identify the PRMs and their levels using standard GC-MS techniques. This would be irrespective of whether they had been added to the fragrance oil as individual chemicals, as components of naturals or from proprietary bases. Although proprietary bases and and naturals are included in our examples, when analyzing a commercially available fragrance composition via GC-MS one could simply identify the components of the base or natural oil as part of the overall fragrance mixture and their levels, without being able to identify which proprietary base or natural oil the PRM had come from.

The nature and type of PRMs in the fragrance compositions according to the present invention can be selected by the skilled person, on the basis of its general knowledge together with the teachings contained herein, with reference to the intended use or application of the fragrance composition and the desired fragrance profile effect. Non-limiting examples of suitable PRMs are disclosed in U.S. Pat. No. 4,145,184, U.S. Pat. No. 4,209,417, U.S. Pat. No. 4,515,705 and U.S. Pat. No. 4,152,272.

Preferably, the fragrance composition comprises a perfume raw material, wherein the perfume raw material comprises at least one highly volatile perfume raw material having a vapour pressure greater than or equal to 0.001 Torr (≧0.00013 kPa) at 25° C. and the highly volatile perfume raw material is present in an amount from about 0.001 wt % to about 99.9 wt %, preferably from about 0.01 wt % to about 99 wt %, relative to the total weight of the fragrance composition. Preferably, the fragrance composition comprises at least 2, 3, 4, 5, 6 or more highly volatile perfume raw materials having a vapour pressure greater than or equal to 0.001 Torr (≧0.00013 kPa) at 25° C.

Preferably non-limiting examples of highly volatile perfume raw materials are listed in Table 1.

TABLE 1
Highly Volatile Perfume Raw Materials for Use in the Fragrance Compositions

| | | | Vapour |
|---|---|---|---|
| CAS | | | Pressure/Torr |
| Number | Chemical Name | Common Name** | at 25° C.*^{§} |
| 107-31-3 | Formic acid, methyl ester | Methyl Formate | 732.00000000 |
| 75-18-3 | Methane, 1,1′-thiobis- | Dimethyl Sulfide 1.0% | 647.00000000 |
| | | In DEP | |
| 141-78-6 | Acetic acid ethyl ester | Ethyl Acetate | 112.00000000 |
| 105-37-3 | Propanoic acid, ethyl ester | Ethyl Propionate | 44.50000000 |
| 110-19-0 | Acetic acid, 2-methylpropyl | Isobutyl Acetate | 18.00000000 |
| | ester | | |
| 105-54-4 | Butanoic acid, ethyl ester | Ethyl Butyrate | 13.90000000 |
| 14765-30-1 | 1-Butanol | Butyl Alcohol | 8.52000000 |
| 7452-79-1 | Butanoic acid, 2-methyl-, ethyl | Ethyl-2-Methyl Butyrate | 7.85000000 |
| | ester | | |
| 123-92-2 | 1-Butanol, 3-methyl-, 1-acetate | Iso Amyl Acetate | 5.68000000 |
| 66576-71-4 | Butanoic acid, 2-methyl-, 1- | Iso Propyl 2- | 5.10000000 |
| | methylethyl ester | Methylbutyrate | |
| 110-43-0 | 2-Heptanone | Methyl Amyl Ketone | 4.73000000 |
| 6728-26-3 | 2-Hexenal, (2E)- | Trans-2 Hexenal | 4.62000000 |
| 123-51-3 | 1-Butanol, 3-methyl- | Isoamyl Alcohol | 4.16000000 |
| 1191-16-8 | 2-Buten-1-ol, 3-methyl-, 1- | Prenyl acetate | 3.99000000 |
| | acetate | | |
| 57366-77-5 | 1,3-Dioxolane-2-methanamine, | Methyl Dioxolan | 3.88000000 |
| | N-methyl- | | |
| 7785-70-8 | Bicyclo[3.1.1]hept-2-ene, 2,6,6- | Alpha Pinene | 3.49000000 |
| | trimethyl-, (1R,5R)- | | |
| 79-92-5 | Bicyclo[2.2.1]heptane, 2,2- | Camphene | 3.38000000 |
| | dimethyl-3-methylene- | | |
| 94087-83-9 | 2-Butanethiol, 4-methoxy-2- | 4-Methoxy-2-Methyl-2- | 3.31000000 |
| | methyl- | Butanenthiol | |
| 39255-32-8 | Pentanoic acid, 2-methyl-, ethyl | Manzanate | 2.91000000 |
| | ester | | |
| 3387-41-5 | Bicyclo[3.1.0]hexane, 4- | Sabinene | 2.63000000 |
| | methylene-1-(1-methylethyl)- | | |
| 127-91-3 | Bicyclo[3.1.1]heptane, 6,6- | Beta Pinene | 2.40000000 |
| | dimethyl-2-methylene- | | |
| 105-68-0 | 1-Butanol, 3-methyl-, 1- | Amyl Propionate | 2.36000000 |
| | propanoate | | |
| 123-35-3 | 1,6-Octadiene, 7-methyl-3- | Myrcene | 2.29000000 |
| | methylene- | | |
| 124-13-0 | Octanal | Octyl Aldehyde | 2.07000000 |
| 7392-19-0 | 2H-Pyran, 2-ethenyltetrahydro- | Limetol | 1.90000000 |
| | 2,6,6-trimethyl- | | |
| 111-13-7 | 2-Octanone | Methyl Hexyl Ketone | 1.72000000 |
| 123-66-0 | Hexanoic acid, ethyl ester | Ethyl Caproate | 1.66000000 |
| 470-82-6 | 2-Oxabicyclo[2.2.2]octane, 1,3, | Eucalyptol | 1.65000000 |
| | 3-trimethyl- | | |
| 99-87-6 | Benzene, 1-methyl-4-(1- | Para Cymene | 1.65000000 |
| | methylethyl)- | | |
| 104-93-8 | Benzene, 1-methoxy-4-methyl- | Para Cresyl Methyl | 1.65000000 |
| | | Ether | |
| 13877-91-3 | 1,3,6-Octatriene, 3,7-dimethyl- | Ocimene | 1.56000000 |
| 138-86-3 | Cyclohexene, 1-methyl-4-(1- | dl-Limonene | 1.54000000 |
| | methylethenyl)- | | |
| 5989-27-5 | Cyclohexene, 1-methyl-4-(1- | d-limonene | 1.54000000 |
| | methylethenyl)-, (4R)- | | |
| 106-68-3 | 3-Octanone | Ethyl Amyl Ketone | 1.50000000 |
| 110-41-8 | Undecanal, 2-methyl- | Methyl Nonyl | 1.43000000 |
| | | Acetaldehyde | |
| 142-92-7 | Acetic acid, hexyl ester | Hexyl acetate | 1.39000000 |
| 110-93-0 | 5-Hepten-2-one, 6-methyl- | Methyl Heptenone | 1.28000000 |
| 81925-81-7 | 2-Hepten-4-one, 5-methyl- | Filbertone 1% in TEC | 1.25000000 |
| 3681-71-8 | 3-Hexen-1-ol, 1-acetate, (3Z)- | cis-3-Hexenyl acetate | 1.22000000 |
| 97-64-3 | Propanoic acid, 2-hydroxy-, | Ethyl Lactate | 1.16000000 |
| | ethyl ester | | |
| 586-62-9 | Cyclohexene, 1-methyl-4-(1- | Terpineolene | 1.13000000 |
| | methylethylidene)- | | |
| 51115-64-1 | Butanoic acid, 2-methylbutyl | Amyl butyrate | 1.09000000 |
| | ester | | |
| 106-27-4 | Butanoic acid, 3-methylbutyl | Amyl Butyrate | 1.09000000 |
| | ester | | |
| 99-85-4 | 1,4-Cyclohexadiene, 1-methyl- | Gamma Terpinene | 1.08000000 |
| | 4-(1-methylethyl)- | | |
| 18640-74-9 | Thiazole, 2-(2-methylpropyl)- | 2-Isobutylthiazole | 1.07000000 |
| 928-96-1 | 3-Hexen-1-ol, (3Z)- | cis-3-Hexenol | 1.04000000 |
| 100-52-7 | Benzaldehyde | Benzaldehyde | 0.97400000 |
| 141-97-9 | Butanoic acid, 3-oxo-, ethyl | Ethyl Acetoacetate | 0.89000000 |
| | ester | | |
| 928-95-0 | 2-Hexen-1-ol, (2E)- | Trans-2-Hexenol | 0.87300000 |
| 928-94-9 | 2-Hexen-1-ol, (2Z)- | Beta Gamma Hexenol | 0.87300000 |
| 24691-15-4 | Cyclohexane, 3-ethoxy-1,1,5- | Herbavert | 0.85200000 |
| | trimethyl-, cis-(9CI) | | |
| 19872-52-7 | 2-Pentanone, 4-mercapto-4- | 4-Methyl-4- | 0.84300000 |
| | methyl- | Mercaptopentan-2-one | |
| | | 1 ppm TEC | |
| 3016-19-1 | 2,4,6-Octatriene, 2,6-dimethyl-, | Allo-Ocimene | 0.81600000 |
| | (4E,6E)- | | |
| 69103-20-4 | Oxirane, 2,2-dimethyl-3-(3- | Myroxide | 0.80600000 |
| | methyl-2,4-pentadien-1-yl)- | | |
| 189440-77-5 | 4,7-Octadienoic acid, methyl | Anapear | 0.77700000 |
| | ester, (4E)- | | |
| 67633-96-9 | Carbonic acid, (3Z)-3-hexen-1- | Liffarome ™ | 0.72100000 |
| | yl methyl ester | | |
| 123-68-2 | Hexanoic acid, 2-propen-1-yl | Allyl Caproate | 0.67800000 |
| | ester | | |
| 106-72-9 | 5-Heptenal, 2,6-dimethyl- | Melonal | 0.62200000 |
| 106-30-9 | Heptanoic acid, ethyl ester | Ethyl Oenanthate | 0.60200000 |
| 68039-49-6 | 3-Cyclohexene-1- | Ligustral or Triplal | 0.57800000 |
| | carboxaldehyde, 2,4-dimethyl- | | |
| 101-48-4 | Benzene, (2,2-dimethoxyethyl)- | Phenyl Acetaldehyde | 0.55600000 |
| | | Dimethyl Acetal | |
| 16409-43-1 | 2H-Pyran, tetrahydro-4-methyl- | Rose Oxide | 0.55100000 |
| | 2-(2-methyl-1-propen-1-yl)- | | |
| 925-78-0 | 3-Nonanone | Ethyl Hexyl Ketone | 0.55100000 |
| 100-47-0 | Benzonitrile | Benzyl Nitrile | 0.52400000 |
| 589-98-0 | 3-Octanol | Octanol-3 | 0.51200000 |
| 58430-94-7 | 1-Hexanol, 3,5,5-trimethyl-, 1- | Iso Nonyl Acetate | 0.47000000 |
| | acetate | | |
| 10250-45-0 | 4-Heptanol, 2,6-dimethyl-, 4- | Alicate | 0.45400000 |
| | acetate | | |
| 105-79-3 | Hexanoic acid, 2-methylpropyl | Iso Butyl Caproate | 0.41300000 |
| | ester | | |
| 2349-07-7 | Propanoic acid, 2-methyl-, hexyl | Hexyl isobutyrate | 0.41300000 |
| | ester | | |
| 23250-42-2 | Cyclohexanecarboxylic acid, 1, | Cyprissate | 0.40500000 |
| | 4-dimethyl-, methyl ester, trans- | | |
| 122-78-1 | Benzeneacetaldehyde | Phenyl acetaldehyde | 0.36800000 |
| 5405-41-4 | Butanoic acid, 3-hydroxy-, ethyl | Ethyl-3-Hydroxy | 0.36200000 |
| | ester | Butyrate | |
| 105-53-3 | Propanedioic acid, 1,3-diethyl | Diethyl Malonate | 0.34400000 |
| | ester | | |
| 93-58-3 | Benzoic acid, methyl ester | Methyl Benzoate | 0.34000000 |
| 16356-11-9 | 1,3,5-Undecatriene | Undecatriene | 0.33600000 |
| 65405-70-1 | 4-Decenal, (4E)- | Decenal (Trans-4) | 0.33100000 |
| 54546-26-8 | 1,3-Dioxane, 2-butyl-4,4,6- | Herboxane | 0.33000000 |
| | trimethyl- | | |
| 13254-34-7 | 2-Heptanol, 2,6-dimethyl- | Dimethyl-2 6-Heptan-2- | 0.33000000 |
| | | ol | |
| 98-86-2 | Ethanone, 1-phenyl- | Acetophenone | 0.29900000 |
| 93-53-8 | Benzeneacetaldehyde, α-methyl- | Hydratropic aldehyde | 0.29400000 |
| 80118-06-5 | Propanoic acid, 2-methyl-, 1,3- | Iso Pentyrate | 0.28500000 |
| | dimethyl-3-buten-1-yl ester | | |
| 557-48-2 | 2,6-Nonadienal, (2E,6Z)- | E Z-2,6-Nonadien-1-al | 0.28000000 |
| 24683-00-9 | Pyrazine, 2-methoxy-3-(2- | 2-Methoxy-3-Isobutyl | 0.27300000 |
| | methylpropyl)- | Pyrazine | |
| 104-57-4 | Formic acid, phenylmethyl ester | Benzyl Formate | 0.27300000 |
| 104-45-0 | Benzene, 1-methoxy-4-propyl- | Dihydroanethole | 0.26600000 |
| 491-07-6 | Cyclohexanone, 5-methyl-2-(1- | Iso Menthone | 0.25600000 |
| | methylethyl)-, (2R,5R)-rel- | | |
| 89-80-5 | Cyclohexanone, 5-methyl-2-(1- | Menthone Racemic | 0.25600000 |
| | methylethyl)-, (2R,5S)-rel- | | |
| 2463-53-8 | 2-Nonenal | 2 Nonen-1-al | 0.25600000 |
| 55739-89-4 | Cyclohexanone, 2-ethyl-4,4- | Thuyacetone | 0.25000000 |
| | dimethyl- | | |
| 150-78-7 | Benzene, 1,4-dimethoxy- | Hydroquinone Dimethyl | 0.25000000 |
| | | Ether | |
| 64988-06-3 | Benzene, 1-(ethoxymethyl)-2- | Rosacene | 0.24600000 |
| | methoxy- | | |
| 76-22-2 | Bicyclo[2.2.1]heptan-2-one, 1,7, | Camphor gum | 0.22500000 |
| | 7-trimethyl- | | |
| 67674-46-8 | 2-Hexene, 6,6-dimethoxy-2,5,5- | Methyl Pamplemousse | 0.21400000 |
| | trimethyl- | | |
| 112-31-2 | Decanal | Decyl Aldehyde | 0.20700000 |
| 16251-77-7 | Benzenepropanal, β-methyl- | Trifernal | 0.20600000 |
| 93-92-5 | Benzenemethanol, α-methyl-, 1- | Methylphenylcarbinol | 0.20300000 |
| | acetate | Acetate | |
| 143-13-5 | Acetic acid, nonyl ester | Nonyl Acetate | 0.19700000 |
| 122-00-9 | Ethanone, 1-(4-methylphenyl)- | Para Methyl | 0.18700000 |
| | | Acetophenone | |
| 24237-00-1 | 2H-Pyran, 6-butyl-3,6-dihydro- | Gyrane | 0.18600000 |
| | 2,4-dimethyl- | | |
| 41519-23-7 | Propanoic acid, 2-methyl-, (3Z)- | Hexenyl Isobutyrate | 0.18200000 |
| | 3-hexen-1-yl ester | | |
| 93-89-0 | Benzoic acid, ethyl ester | Ethyl Benzoate | 0.18000000 |
| 20780-48-7 | 3-Octanol, 3,7-dimethyl-, 3- | Tetrahydro Linalyl | 0.18000000 |
| | acetate | Acetate | |
| 101-41-7 | Methyl 2-phenylacetate | Methylphenyl acetate | 0.17600000 |
| 40853-55-2 | 1-Hexanol, 5-methyl-2-(1- | Tetrahydro Lavandulyl | 0.17300000 |
| | methylethyl)-, 1-acetate | Acetate | |
| 933-48-2 | Cyclohexanol, 3,3,5-trimethyl-, | Trimethylcyclohexanol | 0.17300000 |
| | (1R,5R)-rel- | | |
| 35158-25-9 | 2-Hexenal, 5-methyl-2-(1- | Lactone of Cis Jasmone | 0.17200000 |
| | methylethyl)- | | |
| 18479-58-8 | 7-Octen-2-ol, 2,6-dimethyl- | Dihydromyrcenol | 0.16600000 |
| 140-11-4 | Acetic acid, phenylmethyl ester | Benzyl acetate | 0.16400000 |
| 14765-30-1 | Cyclohexanone, 2-(1- | 2-sec-Butyl Cyclo | 0.16300000 |
| | methylpropyl)- | Hexanone | |
| 20125-84-2 | 3-Octen-1-ol, (3Z)- | Octenol | 0.16000000 |
| 142-19-8 | Heptanoic acid, 2-propen-1-yl | Allyl Heptoate | 0.16000000 |
| | ester | | |
| 100-51-6 | Benzenemethanol | Benzyl Alcohol | 0.15800000 |
| 10032-15-2 | Butanoic acid, 2-methyl-, hexyl | Hexyl-2-Methyl | 0.15800000 |
| | ester | Butyrate | |
| 695-06-7 | 2(3H)-Furanone, 5-ethyldihydro- | Gamma Hexalactone | 0.15200000 |
| 21722-83-8 | Cyclohexaneethanol, 1-acetate | Cyclohexyl Ethyl | 0.15200000 |
| | | Acetate | |
| 111-79-5 | 2-Nonenoic acid, methyl ester | Methyl-2-Nonenoate | 0.14600000 |
| 16491-36-4 | Butanoic acid, (3Z)-3-hexen-1-yl | Cis 3 Hexenyl Butyrate | 0.13500000 |
| | ester | | |
| 111-12-6 | 2-Octynoic acid, methyl ester | Methyl Heptine | 0.12500000 |
| | | Carbonate | |
| 59323-76-1 | 1,3-Oxathiane, 2-methyl-4- | Oxane | 0.12300000 |
| | propyl-, (2R,4S)-rel- | | |
| 62439-41-2 | Heptanal, 6-methoxy-2,6- | Methoxy Melonal | 0.11900000 |
| | dimethyl- | | |
| 13851-11-1 | Bicyclo[2.2.1]heptan-2-ol, 1,3,3- | Fenchyl Acetate | 0.11700000 |
| | trimethyl-, 2-acetate | | |
| 115-95-7 | 1,6-Octadien-3-ol, 3,7-dimethyl-, | Linalyl acetate | 0.11600000 |
| | 3-acetate | | |
| 18479-57-7 | 2-Octanol, 2,6-dimethyl- | Tetra-Hydro Myrcenol | 0.11500000 |
| 78-69-3 | 3,7-dimethyloctan-3-ol | Tetra-Hydro Linalool | 0.11500000 |
| 111-87-5 | 1-Octanol | Octyl Alcohol | 0.11400000 |
| 71159-90-5 | 3-Cyclohexene-1-methanethiol, | Grapefruit mercaptan | 0.10500000 |
| | α,α,4-trimethyl- | | |
| 80-25-1 | Cyclohexanemethanol, α,α,4- | Menthanyl Acetate | 0.10300000 |
| | trimethyl-, 1-acetate | | |
| 88-41-5 | Cyclohexanol, 2-(1,1- | Verdox ™ | 0.10300000 |
| | dimethylethyl)-, 1-acetate | | |
| 32210-23-4 | Cyclohexanol, 4-(1,1- | Vertenex | 0.10300000 |
| | dimethylethyl)-, 1-acetate | | |
| 112-44-7 | Undecanal | n-Undecanal | 0.10200000 |
| 24168-70-5 | Pyrazine, 2-methoxy-3-(1- | Methoxyisobutylpyrazine | 0.09950000 |
| | methylpropyl)- | | |
| 89-79-2 | Cyclohexanol, 5-methyl-2-(1- | Iso-Pulegol | 0.09930000 |
| | methylethenyl)-, (1R,2S,5R)- | | |
| 112-12-9 | 2-Undecanone | Methyl Nonyl Ketone | 0.09780000 |
| 103-05-9 | Benzenepropanol, α,α-dimethyl- | Phenyl Ethyl Dimethyl | 0.09770000 |
| | | Carbinol | |
| 125-12-2 | Bicyclo[2.2.1]heptan-2-ol, 1,7,7- | Iso Bornyl Acetate | 0.09590000 |
| | trimethyl-, 2-acetate, (1R,2R,4R)- | | |
| | rel- | | |
| 78-70-6 | 1,6-Octadien-3-ol, 3,7-dimethyl- | Linalool | 0.09050000 |
| 101-97-3 | Benzeneacetic acid, ethyl ester | Ethyl Phenyl Acetate | 0.08970000 |
| 100-86-7 | Benzeneethanol, α,α-dimethyl- | Dimethyl Benzyl | 0.08880000 |
| | | Carbinol | |
| 188570-78-7 | Cyclopropanecarboxylic acid, | Montaverdi | 0.08640000 |
| | (3Z)-3-hexen-1-yl ester | | |
| 67634-25-7 | 3-Cyclohexene-1-methanol, 3,5- | Floralate | 0.08500000 |
| | dimethyl-, 1-acetate | | |
| 112-44-7 | Undecanal | Undecyl Aldehyde | 0.08320000 |
| 32669-00-4 | Ethanone, 1-(3-cycloocten-1-yl)- | Tanaisone ® | 0.08150000 |
| 98-53-3 | Cyclohexanone, 4-(1,1- | Patchi | 0.07780000 |
| | dimethylethyl)- | | |
| 35854-86-5 | 6-Nonen-1-ol, (6Z)- | cis-6-None-1-ol | 0.07770000 |
| 5331-14-6 | Benzene, (2-butoxyethyl)- | Butyl phenethyl ether | 0.07760000 |
| 80-57-9 | Bicyclo[3.1.1]hept-3-en-2-one, | Verbenone | 0.07730000 |
| | 4,6,6-trimethyl- | | |
| 22471-55-2 | Cyclohexanecarboxylic acid, 2, | Thesaron | 0.07670000 |
| | 2,6-trimethyl-, ethyl ester, (1R, | | |
| | 6S)-rel- | | |
| 60-12-8 | 2-phenyl ethanol | Phenethyl alcohol or | 0.07410000 |
| | | Phenylethyl alcohol | |
| 106-26-3 | 2,6-Octadienal, 3,7-dimethyl-, | Neral | 0.07120000 |
| | (2Z)- | | |
| 5392-40-5 | 2,6-Octadienal, 3,7-dimethyl- | Citral | 0.07120000 |
| 89-48-5 | Cyclohexanol, 5-methyl-2-(1- | Menthyl Acetate | 0.07070000 |
| | methylethyl)-, 1-acetate, (1R,2S, | | |
| | 5R)-rel- | | |
| 119-36-8 | Benzoic acid, 2-hydroxy-, | Methyl salicylate | 0.07000000 |
| | methyl ester | | |
| 4180-23-8 | Benzene, 1-methoxy-4-(1E)-1- | Anethol | 0.06870000 |
| | propen-1-yl- | | |
| 7549-37-3 | 2,6-Octadiene, 1,1-dimethoxy-3, | Citral Dimethyl Acetal | 0.06780000 |
| | 7-dimethyl- | | |
| 25225-08-5 | Cyclohexanemethanol, α,3,3- | Aphermate | 0.06780000 |
| | trimethyl-, 1-formate | | |
| 3913-81-3 | 2-Decenal, (2E)- | 2-Decene-1-al | 0.06740000 |
| 15373-31-6 | 3-Cyclopentene-1-acetonitrile, 2, | Cantryl ® | 0.06700000 |
| | 2,3-trimethyl- | | |
| 6485-40-1 | 2-Cyclohexen-1-one, 2-methyl- | Laevo carvone | 0.06560000 |
| | 5-(1-methylethenyl)-, (5R)- | | |
| 16587-71-6 | Cyclohexanone, 4-(1,1- | Orivone | 0.06490000 |
| | dimethylpropyl)- | | |
| 62406-73-9 | 6,10-Dioxaspiro[4.5]decane, | Opalal CI | 0.06290000 |
| | 8,8-dimethyl-7-(1-methylethyl)- | | |
| 3720-16-9 | 2-Cyclohexen-1-one, 3-methyl- | Livescone | 0.06270000 |
| | 5-propyl- | | |
| 13816-33-6 | Benzonitrile, 4-(1-methylethyl)- | Cumin Nitrile | 0.06230000 |
| 67019-89-0 | 2,6-Nonadienenitrile | Violet Nitrile | 0.06200000 |
| 53398-85-9 | Butanoic acid, 2-methyl-, (3Z)- | cis-3-Hexenyl Alpha | 0.06130000 |
| | 3-hexen-1-yl ester | Methyl Butyrate | |
| 208041-98-9 | Heptanenitrile, 2-propyl- | Jasmonitrile | 0.05920000 |
| 16510-27-3 | Benzene, 1-(cyclopropylmethyl)- | Toscanol | 0.05870000 |
| | 4-methoxy- | | |
| 111-80-8 | 2-Nonynoic acid, methyl ester | Methyl Octine | 0.05680000 |
| | | Carbonate | |
| 103-45-7 | Acetic acid, 2-phenylethyl ester | Phenyl Ethyl Acetate | 0.05640000 |
| 2550-26-7 | 2-Butanone, 4-phenyl- | Benzyl Acetone | 0.05570000 |
| 13491-79-7 | Cyclohexanol, 2-(1,1- | Verdol | 0.05430000 |
| | dimethylethyl)- | | |
| 7786-44-9 | 2,6-Nonadien-1-ol | 2,6-Nonadien-1-ol | 0.05370000 |
| 103-28-6 | Propanoic acid, 2-methyl-, | Benzyl Iso Butyrate | 0.05130000 |
| | phenylmethyl ester | | |
| 104-62-1 | Formic acid, 2-phenylethyl ester | Phenyl Ethyl Formate | 0.05050000 |
| 28462-85-3 | Bicyclo[2.2.1]heptan-2-ol, 1,2,3, | Humus Ether | 0.04870000 |
| | 3-tetramethyl-, (1R,2R,4S)-rel- | | |
| 122-03-2 | Benzaldehyde, 4-(1-methylethyl)- | Cuminic Aldehyde | 0.04820000 |
| 358331-95-0 | 2,5-Octadien-4-one, 5,6,7- | Pomarose | 0.04810000 |
| | trimethyl-, (2E)- | | |
| 562-74-3 | 3-Cyclohexen-1-ol, 4-methyl-1- | Terpinenol-4 | 0.04780000 |
| | (1-methylethyl)- | | |
| 68527-77-5 | 3-Cyclohexene-1-methanol, 2,4, | Isocyclogeraniol | 0.04640000 |
| | 6-trimethyl- | | |
| 35852-46-1 | Pentanoic acid, (3Z)-3-hexen-1- | Cis-3-Hexenyl Valerate | 0.04580000 |
| | yl ester | | |
| 2756-56-1 | Bicyclo[2.2.1]heptan-2-ol, 1,7,7- | Iso Bornyl Propionate | 0.04540000 |
| | trimethyl-, 2-propanoate, (1R, | | |
| | 2R,4R)-rel- | | |
| 14374-92-6 | Benzene, 1-methyl-4-(1- | Verdoracine | 0.04460000 |
| | methylethyl)-2-(1-propen-1-yl)- | | |
| 6784-13-0 | 3-Cyclohexene-1-propanal, β,4- | Limonenal | 0.04380000 |
| | dimethyl- | | |
| 8000-41-7 | 2-(4-methyl-1-cyclohex-3- | Alpha Terpineol | 0.04320000 |
| | enyl)propan-2-ol | | |
| 41884-28-0 | 1-Hexanol, 5-methyl-2-(1- | Tetrahydro Lavandulol | 0.04230000 |
| | methylethyl)-, (2R)- | | |
| 22457-23-4 | 3-Heptanone, 5-methyl-, oxime | Stemone ® | 0.04140000 |
| 104-50-7 | 2(3H)-Furanone, 5- | Gamma Octalactone | 0.04080000 |
| | butyldihydro- | | |
| 143-08-8 | 1-Nonanol | Nonyl Alcohol | 0.04070000 |
| 3613-30-7 | Octanal, 7-methoxy-3,7- | Methoxycitronellal | 0.04020000 |
| | dimethyl- | | |
| 67634-00-8 | Acetic acid, 2-(3-methylbutoxy)-, | Allyl Amyl Glycolate | 0.04000000 |
| | 2-propen-1-yl ester | | |
| 464-45-9 | Bicyclo[2.2.1]heptan-2-ol, 1,7,7- | 1-Borneol | 0.03980000 |
| | trimethyl-, (1S,2R,4S)- | | |
| 124-76-5 | Bicyclo[2.2.1]heptan-2-ol, 1,7,7- | 1.7.7-Trimethyl- | 0.03980000 |
| | trimethyl-, (1R,2R,4R)-rel- | Bicyclo-1.2.2-Heptanol-2 | |
| 67874-72-0 | Cyclohexanol, 2-(1,1- | Coniferan | 0.03980000 |
| | dimethylpropyl)-, 1-acetate | | |
| 80-26-2 | 3-Cyclohexene-1-methanol, α,α, | Terpinyl Acetate | 0.03920000 |
| | 4-trimethyl-, 1-acetate | | |
| 498-81-7 | Cyclohexanemethanol, α,α,4- | Dihydro Terpineol | 0.03920000 |
| | trimethyl- | | |
| 112-45-8 | 10-Undecenal | Undecylenic aldehyde | 0.03900000 |
| 35044-57-6 | 2,4-Cyclohexadiene-1- | Ethyl Safranate | 0.03880000 |
| | carboxylic acid, 2,6,6-trimethyl-, | | |
| | ethyl ester | | |
| 106-21-8 | 1-Octanol, 3,7-dimethyl- | Dimethyl Octanol | 0.03860000 |
| 84560-00-9 | Cyclopentanol, 2-pentyl- | Cyclopentol | 0.03790000 |
| 82461-14-1 | Furan, tetrahydro-2,4-dimethyl- | Rhubafuran ® | 0.03780000 |
| | 4-phenyl- | | |
| 56011-02-0 | Benzene, [2-(3-methylbutoxy) | Phenyl Ethyl Isoamyl | 0.03690000 |
| | ethyl]- | Ether | |
| 103-37-7 | Butanoic acid, phenylmethyl | Benzyl Butyrate | 0.03660000 |
| | ester | | |
| 6378-65-0 | Hexyl hexanoate | Hexyl hexanoate | 0.03490000 |
| 118-61-6 | Benzoic acid, 2-hydroxy-, ethyl | Ethyl salicylate | 0.03480000 |
| | ester | | |
| 98-52-2 | Cyclohexanol, 4-(1,1- | Patchon | 0.03480000 |
| | dimethylethyl)- | | |
| 115-99-1 | 1,6-Octadien-3-ol, 3,7-dimethyl-, | Linalyl Formate | 0.03440000 |
| | 3-formate | | |
| 112-54-9 | Dodecanal | Lauric Aldehyde | 0.03440000 |
| 53046-97-2 | 3,6-Nonadien-1-ol, (3Z,6Z)- | 3,6 Nonadien-1-ol | 0.03360000 |
| 76649-25-7 | 3,6-Nonadien-1-ol | 3,6-Nonadien-1-ol | 0.03360000 |
| 141-25-3 | 3,7-Dimethyloct-6-en-1-ol | Rhodinol | 0.03290000 |
| 1975-78-6 | Decanenitrile | Decanonitrile | 0.03250000 |
| 2216-51-5 | Cyclohexanol, 5-methyl-2-(1- | L-Menthol | 0.03230000 |
| | methylethyl)-, (1R,2S,5R)- | | |
| 3658-77-3 | 4-hydroxy-2,5-dimethylfuran-3- | Pineapple Ketone | 0.03200000 |
| | one | | |
| 103-93-5 | Propanoic acid, 2-methyl-, 4- | Para Cresyl iso-Butyrate | 0.03120000 |
| | methylphenyl ester | | |
| 24717-86-0 | Propanoic acid, 2-methyl-, (1R, | Abierate | 0.03110000 |
| | 2S,4R)-1,7,7- | | |
| | trimethylbicyclo[2.2.1]hept-2-yl | | |
| | ester, rel- | | |
| 67845-46-9 | Acetaldehyde, 2-(4- | Aldehyde XI | 0.03090000 |
| | methylphenoxy)- | | |
| 67883-79-8 | 2-Butenoic acid, 2-methyl-, (3Z)- | Cis-3-Hexenyl Tiglate | 0.03060000 |
| | 3-hexen-1-yl ester, (2E)- | | |
| 33885-51-7 | Bicyclo[3.1.1]hept-2-ene-2- | Pino Acetaldehyde | 0.03040000 |
| | propanal, 6,6-dimethyl- | | |
| 105-85-1 | 6-Octen-1-ol, 3,7-dimethyl-, 1- | Citronellyl Formate | 0.03000000 |
| | formate | | |
| 70214-77-6 | 2-Nonanol, 6,8-dimethyl- | Nonadyl | 0.03010000 |
| 215231-33-7 | Cyclohexanol, 1-methyl-3-(2- | Rossitol | 0.02990000 |
| | methylpropyl)- | | |
| 120-72-9 | 1H-Indole | Indole | 0.02980000 |
| 2463-77-6 | 2-Undecenal | 2-Undecene-1-al | 0.02970000 |
| 675-09-2 | 2H-Pyran-2-one, 4,6-dimethyl- | Levistamel | 0.02940000 |
| 98-55-5 | 3-Cyclohexene-1-methanol, α,α, | Alpha-Terpineol | 0.02830000 |
| | 4-trimethyl- | | |
| 81786-73-4 | 3-Hepten-2-one, 3,4,5,6,6- | Koavone | 0.02750000 |
| | pentamethyl-, (3Z)- | | |
| 122-97-4 | Benzenepropanol | Phenyl Propyl Alcohol | 0.02710000 |
| 39212-23-2 | 2(3H)-Furanone, 5- | Methyl Octalactone | 0.02700000 |
| | butyldihydro-4-methyl- | | |
| 53767-93-4 | 7-Octen-2-ol, 2,6-dimethyl-, 2- | Dihydro Terpinyl | 0.02690000 |
| | acetate | Acetate | |
| 35044-59-8 | 1,3-Cyclohexadiene-1- | Ethyl Safranate | 0.02660000 |
| | carboxylic acid, 2,6,6-trimethyl-, | | |
| | ethyl ester | | |
| 104-55-2 | 2-Propenal, 3-phenyl- | Cinnamic Aldehyde | 0.02650000 |
| 144-39-8 | 1,6-Octadien-3-ol, 3,7-dimethyl-, | Linalyl Propionate | 0.02630000 |
| | 3-propanoate | | |
| 61931-80-4 | 1,6-Nonadien-3-ol, 3,7- | 3,7-Dimethyl-1,6- | 0.02630000 |
| | dimethyl-, 3-acetate | nonadien-3-yl acetate | |
| 102-13-6 | Benzeneacetic acid, 2- | Iso Butyl Phenylacetate | 0.02630000 |
| | methylpropyl ester | | |
| 65443-14-3 | Cyclopentanone, 2,2,5- | Veloutone | 0.02610000 |
| | trimethyl-5-pentyl- | | |
| 141-12-8 | 2,6-Octadien-1-ol, 3,7-dimethyl-, | Neryl Acetate | 0.02560000 |
| | 1-acetate, (2Z)- | | |
| 105-87-3 | 2,6-Octadien-1-ol, 3,7-dimethyl-, | Geranyl acetate | 0.02560000 |
| | 1-acetate, (2E)- | | |
| 68141-17-3 | Undecane, 1,1-dimethoxy-2- | Methyl Nonyl | 0.02550000 |
| | methyl- | Acetaldehyde Dimethyl | |
| | | Acetal | |
| 2206-94-2 | Benzenemethanol, α-methylene-, | Indocolore | 0.02550000 |
| | 1-acetate | | |
| 10528-67-3 | Cyclohexanepropanol, α-methyl- | Cyclohexylmagnol | 0.02550000 |
| 123-11-5 | Benzaldehyde, 4-methoxy- | Anisic Aldehyde | 0.02490000 |
| 57576-09-7 | Cyclohexanol, 5-methyl-2-(1- | Iso Pulegol Acetate | 0.02480000 |
| | methylethenyl)-, 1-acetate, (1R, | | |
| | 2S,5R)- | | |
| 51566-62-2 | 6-Octenenitrile, 3,7-dimethyl- | Citronellyl Nitrile | 0.02470000 |
| 60335-71-9 | 2H-Pyran, 3,6-dihydro-4- | Rosyrane Super | 0.02470000 |
| | methyl-2-phenyl- | | |
| 30385-25-2 | 6-Octen-2-ol, 2,6-dimethyl- | Dihydromyrcenol | 0.02440000 |
| 101-84-8 | Benzene, 1,1′-oxybis- | Diphenyl Oxide | 0.02230000 |
| 136-60-7 | Benzoic acid, butyl ester | Butyl Benzoate | 0.02170000 |
| 93939-86-7 | 5,8-Methano-2H-1-benzopyran, | Rhuboflor | 0.02120000 |
| | 6-ethylideneoctahydro- | | |
| 83926-73-2 | Cyclohexanepropanol, α,α- | Coranol | 0.02100000 |
| | dimethyl- | | |
| 125109-85-5 | Benzenepropanal, β-methyl-3- | Florhydral | 0.02070000 |
| | (1-methylethyl)- | | |
| 104-21-2 | Benzenemethanol, 4-methoxy-, | Anisyl Acetate | 0.02050000 |
| | 1-acetate | | |
| 1365-19-1 | 2-Furanmethanol, 5- | Linalool Oxide | 0.02050000 |
| | ethenyltetrahydro-α,α,5- | | |
| | trimethyl- | | |
| 137-03-1 | Cyclopentanone, 2-heptyl- | Frutalone | 0.02040000 |
| 2563-07-7 | Phenol, 2-ethoxy-4-methyl- | Ultravanil | 0.02030000 |
| 1128-08-1 | 2-Cyclopenten-1-one, 3-methyl- | Dihydrojasmone | 0.02020000 |
| | 2-pentyl- | | |
| 7493-57-4 | Benzene, [2-(1-propoxyethoxy) | Acetaldehyde | 0.01990000 |
| | ethyl]- | | |
| 141-25-3 | 7-Octen-1-ol, 3,7-dimethyl- | Rhodinol | 0.01970000 |
| 216970-21-7 | Bicyclo[4.3.1]decane, 3- | 3-Methoxy-7,7- | 0.01960000 |
| | methoxy-7,7-dimethyl-10- | dimethyl-10- | |
| | methylene- | methylenebicyclo[4.3.1]decane | |
| | | decane | |
| 319002-92-1 | Propanoic acid, 2-(1,1- | Sclareolate ® | 0.01960000 |
| | dimethylpropoxy)-, propyl ester, | | |
| | (2S)- | | |
| 85-91-6 | Benzoic acid, 2-(methylamino)-, | Dimethyl anthranilate | 0.01930000 |
| | methyl ester | | |
| 13828-37-0 | Cyclohexanemethanol, 4-(1- | Mayol | 0.01920000 |
| | methylethyl)-, cis- | | |
| 26330-65-4 | (E)-6-ethyl-3-methyloct-6-en-1- | Super Muguet | 0.01850000 |
| | ol | | |
| 7540-51-4 | 6-Octen-1-ol, 3,7-dimethyl-, | L-Citronellol | 0.01830000 |
| | (3S)- | | |
| 106-22-9 | 6-Octen-1-ol, 3,7-dimethyl- | Citronellol | 0.01830000 |
| 543-39-5 | 7-Octen-2-ol, 2-methyl-6- | Myrcenol | 0.01820000 |
| | methylene- | | |
| 7775-00-0 | Benzenepropanal, 4-(1- | Cyclemax | 0.01820000 |
| | methylethyl)- | | |
| 18479-54-4 | 4,6-Octadien-3-ol, 3,7-dimethyl- | Muguol | 0.01800000 |
| 29214-60-6 | Octanoic acid, 2-acetyl-, ethyl | Gelsone | 0.01790000 |
| | ester | | |
| 1209-61-6 | 5-Oxatricyclo[8.2.0.04,6] | Tobacarol | 0.01730000 |
| | dodecane, 4,9,12,12-tetramethyl- | | |
| 57934-97-1 | 2-Cyclohexene-1-carboxylic | Givescone | 0.01710000 |
| | acid, 2-ethyl-6,6-dimethyl-, | | |
| | ethyl ester | | |
| 14901-07-6 | 3-Buten-2-one, 4-(2,6,6- | Beta-Ionone | 0.01690000 |
| | trimethyl-1-cyclohexen-1-yl)-, | | |
| | (3E)- | | |
| 64001-15-6 | 4,7-Methano-1H-inden-5-ol, | Dihydro Cyclacet | 0.01630000 |
| | octahydro-, 5-acetate | | |
| 95-41-0 | 2-Cyclopenten-1-one, 2-hexyl- | Iso Jasmone T | 0.01600000 |
| 134-20-3 | Benzoic acid, 2-amino-, methyl | Methyl Anthranilate | 0.01580000 |
| | ester | | |
| 100-06-1 | Ethanone, 1-(4-methoxyphenyl)- | Para Methoxy | 0.01550000 |
| | | Acetophenone | |
| 105-86-2 | 2,6-Octadien-1-ol, 3,7-dimethyl-, | Geranyl Formate | 0.01540000 |
| | 1-formate, (2E)- | | |
| 154171-77-4 | Spiro[1,3-dioxolane-2,8′(5′H)- | Ysamber K ® | 0.01470000 |
| | [2H-2,4a]methanonaphthalene], | | |
| | hexahydro-1′,1′,5′,5′-tetramethyl-, | | |
| | (2′S,4′aS,8′aS)-(9CI) | | |
| 154171-76-3 | Spiro[1,3-dioxolane-2,8′(5′H)- | Ysamber | 0.01470000 |
| | [2H-2,4a]methanonaphthalene], | | |
| | hexahydro-1′,1′,5′,5′-tetramethyl- | | |
| 127-41-3 | 3-Buten-2-one, 4-(2,6,6- | Alpha-Ionone | 0.01440000 |
| | trimethyl-2-cyclohexen-1-yl)-, | | |
| | (3E)- | | |
| 151-05-3 | Benzeneethanol, α,α-dimethyl-, | Dimethyl Benzyl | 0.01390000 |
| | 1-acetate | Carbinyl Acetate | |
| 2500-83-6 | 4,7-Methano-1H-inden-5-ol, 3a, | Flor Acetate | 0.01370000 |
| | 4,5,6,7,7a-hexahydro-, 5-acetate | | |
| 150-84-5 | 6-Octen-1-ol, 3,7-dimethyl-, 1- | Citronellyl acetate | 0.01370000 |
| | acetate | | |
| 30310-41-9 | 2H-Pyran, tetrahydro-2-methyl- | Pelargene | 0.01350000 |
| | 4-methylene-6-phenyl- | | |
| 68845-00-1 | Bicyclo[3.3.1]nonane, 2-ethoxy- | Boisiris | 0.01350000 |
| | 2,6,6-trimethyl-9-methylene- | | |
| 106-24-1 | 2,6-Octadien-1-ol, 3,7-dimethyl-, | Geraniol | 0.01330000 |
| | (2E)- | | |
| 106-25-2 | 2,6-Octadien-1-ol, 3,7-dimethyl-, | Nerol | 0.01330000 |
| | (2Z)- | | |
| 75975-83-6 | Bicyclo[7.2.0]undec-4-ene, 4,11, | Vetyvenal | 0.01280000 |
| | 11-trimethyl-8-methylene-, (1R, | | |
| | 4E,9S)- | | |
| 19870-74-7 | 1H-3a,7-Methanoazulene, | Cedryl methyl ether | 0.01280000 |
| | octahydro-6-methoxy-3,6,8,8- | | |
| | tetramethyl-, (3R,3aS,6S,7R,8aS)- | | |
| 87-44-5 | Bicyclo[7.2.0]undec-4-ene, 4,11, | Caryophyllene Extra | 0.01280000 |
| | 11-trimethyl-8-methylene-, (1R, | | |
| | 4E,9S)- | | |
| 54440-17-4 | 1H-Inden-1-one, 2,3-dihydro-2, | Safraleine | 0.01260000 |
| | 3,3-trimethyl- | | |
| 110-98-5 | 2-Propanol, 1,1′-oxybis- | Dipropylene Glycol | 0.01250000 |
| 41890-92-0 | 2-Octanol, 7-methoxy-3,7- | Osyrol ® | 0.01250000 |
| | dimethyl- | | |
| 71077-31-1 | 4,9-Decadienal, 4,8-dimethyl- | Floral Super | 0.01230000 |
| 65-85-0 | Benzoic Acid | Benzoic Acid | 0.01220000 |
| 61444-38-0 | 3-Hexenoic acid, (3Z)-3-hexen- | cis-3-hexenyl-cis-3- | 0.01220000 |
| | 1-yl ester, (3Z)- | hexenoate | |
| 116044-44-1 | Bicyclo[2.2.1]hept-5-ene-2- | Herbanate | 0.01210000 |
| | carboxylic acid, 3-(1- | | |
| | methylethyl)-, ethyl ester, (1R, | | |
| | 2S,3S,4S)-rel- | | |
| 104-54-1 | 2-Propen-1-ol, 3-phenyl- | Cinnamic Alcohol | 0.01170000 |
| 78-35-3 | Propanoic acid, 2-methyl-, 1- | Linalyl Isobutyrate | 0.01170000 |
| | ethenyl-1,5-dimethyl-4-hexen-1- | | |
| | yl ester | | |
| 23495-12-7 | Ethanol, 2-phenoxy-, 1- | Phenoxy Ethyl | 0.01130000 |
| | propanoate | Propionate | |
| 103-26-4 | 2-Propenoic acid, 3-phenyl-, | Methyl Cinnamate | 0.01120000 |
| | methyl ester | | |
| 67634-14-4 | Benzenepropanal, 2-ethyl-α,α- | Florazon (ortho-isomer) | 0.01110000 |
| | dimethyl- | | |
| 5454-19-3 | Propanoic acid, decyl ester | N-Decyl Propionate | 0.01100000 |
| 93-16-3 | Benzene, 1,2-dimethoxy-4-(1- | Methyl Iso Eugenol | 0.01100000 |
| | propen-1-yl)- | | |
| 81782-77-6 | 3-Decen-5-ol, 4-methyl- | 4-Methyl-3-decen-5-ol | 0.01070000 |
| 67845-30-1 | Bicyclo[2.2.2]oct-5-ene-2- | Maceal | 0.01060000 |
| | carboxaldehyde, 6-methyl-8-(1- | | |
| | methylethyl)- | | |
| 97-53-0 | Phenol, 2-methoxy-4-(2-propen- | Eugenol | 0.01040000 |
| | 1-yl)- | | |
| 120-57-0 | 1,3-Benzodioxole-5- | Heliotropin | 0.01040000 |
| | carboxaldehyde | | |
| 93-04-9 | Naphthalene, 2-methoxy- | Beta Naphthyl Methyl | 0.01040000 |
| | | Ether Extra 99 | |
| 4826-62-4 | 2-Dodecenal | 2 Dodecene-1-al | 0.01020000 |
| 20407-84-5 | 2-Dodecenal, (2E)- | Aldehyde Mandarin | 0.01020000 |
| 5462-06-6 | Benzenepropanal, 4-methoxy-α- | Canthoxal | 0.01020000 |
| | methyl- | | |
| 94-60-0 | 1,4-Cyclohexanedicarboxylic | Dimethyl 1,4- | 0.01020000 |
| | acid, 1,4-dimethyl ester | cyclohexanedicarboxylate | |
| 57378-68-4 | 2-Buten-1-one, 1-(2,6,6- | delta-Damascone | 0.01020000 |
| | trimethyl-3-cyclohexen-1-yl)- | | |
| 17283-81-7 | 2-Butanone, 4-(2,6,6-trimethyl- | Dihydro Beta Ionone | 0.01020000 |
| | 1-cyclohexen-1-yl)- | | |
| 1885-38-7 | 2-Propenenitrile, 3-phenyl-, (2E)- | Cinnamalva | 0.01010000 |
| 103-48-0 | Propanoic acid, 2-methyl-, 2- | Phenyl Ethyl Iso | 0.00994000 |
| | phenylethyl ester | Butyrate | |
| 488-10-8 | 2-Cyclopenten-1-one, 3-methyl- | Cis Jasmone | 0.00982000 |
| | 2-(2Z)-2-penten-1-yl- | | |
| 7492-67-3 | Acetaldehyde, 2-[(3,7-dimethyl- | Citronellyloxyacetaldehyde | 0.00967000 |
| | 6-octen-1-yl)oxy]- | | |
| 68683-20-5 | 1-Cyclohexene-1-ethanol, 4-(1- | Iso Bergamate | 0.00965000 |
| | methylethyl)-, 1-formate | | |
| 3025-30-7 | 2,4-Decadienoic acid, ethyl | Ethyl 2,4-Decadienoate | 0.00954000 |
| | ester, (2E,4Z)- | | |
| 103-54-8 | 2-Propen-1-ol, 3-phenyl-, 1- | Cinnamyl Acetate | 0.00940000 |
| | acetate | | |
| 18127-01-0 | Benzenepropanal, 4-(1,1- | Bourgeonal | 0.00934000 |
| | dimethylethyl)- | | |
| 3738-00-9 | Naphtho[2,1-b]furan, | Ambrox ® or Cetalox ® or | 0.00934000 |
| | dodecahydro-3a,6,6,9a- | Synambran | |
| | tetramethyl- | | |
| 51519-65-4 | 1,4-Methanonaphthalen-5(1H)- | Tamisone | 0.00932000 |
| | one, 4,4a,6,7,8,8a-hexahydro- | | |
| 148-05-1 | Dodecanoic acid, 12-hydroxy-, | Dodecalactone | 0.00931000 |
| | λ-lactone (6CI,7CI); 1,12- | | |
| 6790-58-5 | (3aR,5aS,9aS,9bR)-3a,6,6,9a- | Ambronat ® or | 0.00930000 |
| | tetramethyl-2,4,5,5a,7,8,9,9b- | Ambroxan ® | |
| | octahydro-1H- | | |
| | benzo[e][1]benzofuran | | |
| 86-26-0 | 1,1′-Biphenyl, 2-methoxy- | Methyl Diphenyl Ether | 0.00928000 |
| 68738-94-3 | 2-Naphthalenecarboxaldehyde, | Cyclomyral ® | 0.00920000 |
| | octahydro-8,8-dimethyl | | |
| 2705-87-5 | Cyclohexanepropanoic acid, 2- | Allyl Cyclohexane | 0.00925000 |
| | propen-1-yl ester | Propionate | |
| 7011-83-8 | 2(3H)-Furanone, 5- | Lactojasmone ® | 0.00885000 |
| | hexyldihydro-5-methyl- | | |
| 61792-11-8 | 2,6-Nonadienenitrile, 3,7- | Lemonile ® | 0.00884000 |
| | dimethyl- | | |
| 692-86-4 | 10-Undecenoic acid, ethyl ester | Ethyl Undecylenate | 0.00882000 |
| 103-95-7 | Benzenepropanal,α-methyl-4- | Cymal | 0.00881000 |
| | (1-methylethyl)- | | |
| 13019-22-2 | 9-Decen-1-ol | Rosalva | 0.00879000 |
| 94201-19-1 | 1-Oxaspiro[4.5]decan-2-one, 8- | Methyl Laitone 10% | 0.00872000 |
| | methyl- | TEC | |
| 104-61-0 | 2(3H)-Furanone, dihydro-5- | γ-Nonalactone | 0.00858000 |
| | pentyl- | | |
| 706-14-9 | 2(3H)-Furanone, 5- | γ-Decalactone | 0.00852000 |
| | hexyldihydro- | | |
| 24720-09-0 | 2-Buten-1-one, 1-(2,6,6- | α-Damascone | 0.00830000 |
| | trimethyl-2-cyclohexen-1-yl)-, | | |
| | (2E)- | | |
| 39872-57-6 | 2-Buten-1-one, 1-(2,4,4- | Isodamascone | 0.00830000 |
| | trimethyl-2-cyclohexen-1-yl)-, | | |
| | (2E)- | | |
| 705-86-2 | 2H-Pyran-2-one, tetrahydro-6- | Decalactone | 0.00825000 |
| | pentyl- | | |
| 67634-15-5 | Benzenepropanal, 4-ethyl-α,α- | Floralozone | 0.00808000 |
| | dimethyl- | | |
| 40527-42-2 | 1,3-Benzodioxole, 5- | Heliotropin Diethyl | 0.00796000 |
| | (diethoxymethyl)- | Acetal | |
| 56973-85-4 | 4-Penten-1-one, 1-(5,5- | Neobutenone α | 0.00763000 |
| | dimethyl-1-cyclohexen-1-yl)- | | |
| 128-51-8 | Bicyclo[3.1.1]hept-2-ene-2- | Nopyl Acetate | 0.00751000 |
| | ethanol, 6,6-dimethyl-, 2-acetate | | |
| 103-36-6 | 2-Propenoic acid, 3-phenyl-, | Ethyl Cinnamate | 0.00729000 |
| | ethyl ester | | |
| 5182-36-5 | 1,3-Dioxane, 2,4,6-trimethyl-4- | Floropal ® | 0.00709000 |
| | phenyl- | | |
| 42604-12-6 | Cyclododecane, | Boisambrene | 0.00686000 |
| | (methoxymethoxy)- | | |
| 33885-52-8 | Bicyclo[3.1.1]hept-2-ene-2- | Pinyl Iso Butyrate Alpha | 0.00685000 |
| | propanal, α,α,6,6-tetramethyl- | | |
| 92015-65-1 | 2(3H)-Benzofuranone, | Natactone | 0.00680000 |
| | hexahydro-3,6-dimethyl- | | |
| 63767-86-2 | Cyclohexanemethanol, α- | Mugetanol | 0.00678000 |
| | methyl-4-(1-methylethyl)- | | |
| 3288-99-1 | Benzeneacetonitrile, 4-(1,1- | Marenil CI | 0.00665000 |
| | dimethylethyl)- | | |
| 35044-68-9 | 2-Buten-1-one, 1-(2,6,6- | beta-Damascone | 0.00655000 |
| | trimethyl-1-cyclohexen-1-yl)- | | |
| 41724-19-0 | 1,4-Methanonaphthalen-6(2H)- | Plicatone | 0.00652000 |
| | one, octahydro-7-methyl- | | |
| 75147-23-8 | Bicyclo[3.2.1]octan-8-one, 1,5- | Buccoxime ® | 0.00647000 |
| | dimethyl-, oxime | | |
| 25634-93-9 | 2-Methyl-5-phenylpentan-1-ol | Rosaphen ® 600064 | 0.00637000 |
| 55066-48-3 | 3-Methyl-5-phenylpentanol | Phenyl Hexanol | 0.00637000 |
| 495-62-5 | Cyclohexene, 4-(1,5-dimethyl-4- | Bisabolene | 0.00630000 |
| | hexen-1-ylidene)-1-methyl- | | |
| 2785-87-7 | Phenol, 2-methoxy-4-propyl- | Dihydro Eugenol | 0.00624000 |
| 87-19-4 | Benzoic acid, 2-hydroxy-, 2- | Iso Butyl Salicylate | 0.00613000 |
| | methylpropyl ester | | |
| 4430-31-3 | 2H-1-Benzopyran-2-one, | Octahydro Coumarin | 0.00586000 |
| | octahydro- | | |
| 38462-22-5 | Cyclohexanone, 2-(1-mercapto- | Ringonol 50 TEC | 0.00585000 |
| | 1-methylethyl)-5-methyl- | | |
| 77-83-8 | 2-Oxiranecarboxylic acid, 3- | Ethyl Methyl | 0.00571000 |
| | methyl-3-phenyl-, ethyl ester | Phenyl Glycidate | |
| 37677-14-8 | 3-Cyclohexene-1- | Iso Hexenyl | 0.00565000 |
| | carboxaldehyde, 4-(4-methyl-3- | Cyclohexenyl | |
| | penten-1-yl)- | Carboxaldehyde | |
| 103-60-6 | Propanoic acid, 2-methyl-, 2- | Phenoxy Ethyl iso- | 0.00562000 |
| | phenoxyethyl ester | Butyrate | |
| 18096-62-3 | Indeno[1,2-d]-1,3-dioxin, 4,4a,5, | Indoflor ® | 0.00557000 |
| | 9b-tetrahydro- | | |
| 63500-71-0 | 2H-Pyran-4-ol, tetrahydro-4- | Florosa Q | 0.00557000 |
| | methyl-2-(2-methylpropyl)- | | |
| 65405-84-7 | Cyclohexanebutanal, α,2,6,6- | Cetonal ® | 0.00533000 |
| | tetramethyl- | | |
| 171102-41-3 | 4,7-Methano-1H-inden-6-ol, 3a, | Flor Acetate | 0.00530000 |
| | 4,5,6,7,7a-hexahydro-8,8- | | |
| | dimethyl-, 6-acetate | | |
| 10339-55-6 | 1,6-Nonadien-3-ol, 3,7- | Ethyl linalool | 0.00520000 |
| | dimethyl- | | |
| 23267-57-4 | 3-Buten-2-one, 4-(2,2,6- | Ionone Epoxide Beta | 0.00520000 |
| | trimethyl-7-oxabicyclo[4.1.0] | | |
| | hept-1-yl)- | | |
| 97-54-1 | Phenol, 2-methoxy-4-(1-propen- | Isoeugenol | 0.00519000 |
| | 1-yl)- | | |
| 67663-01-8 | 2(3H)-Furanone, 5- | Peacholide | 0.00512000 |
| | hexyldihydro-4-methyl- | | |
| 33885-52-8 | Bicyclo[3.1.1]hept-2-ene-2- | Pinyl Iso Butyrate Alpha | 0.00512000 |
| | propanal, α,α,6,6-tetramethyl- | | |
| 23696-85-7 | 2-Buten-1-one, 1-(2,6,6- | Damascenone | 0.00503000 |
| | trimethyl-1,3-cyclohexadien-1- | | |
| | yl)- | | |
| 80-71-7 | 2-Cyclopenten-1-one, 2- | Maple Lactone | 0.00484000 |
| | hydroxy-3-methyl- | | |
| 67662-96-8 | Propanoic acid, 2,2-dimethyl-, 2- | Pivarose Q | 0.00484000 |
| | phenylethyl ester | | |
| 2437-25-4 | Dodecanenitrile | Clonal | 0.00480000 |
| 141-14-0 | 6-Octen-1-ol, 3,7-dimethyl-, 1- | Citronellyl Propionate | 0.00469000 |
| | propanoate | | |
| 54992-90-4 | 3-Buten-2-one, 4-(2,2,3,6- | Myrrhone | 0.00460000 |
| | tetramethylcyclohexyl)- | | |
| 55066-49-4 | Benzenepentanal, β-methyl- | Mefranal | 0.00455000 |
| 7493-74-5 | Acetic acid, 2-phenoxy-, 2- | Allyl Phenoxy Acetate | 0.00454000 |
| | propen-1-yl ester | | |
| 80-54-6 | Benzenepropanal, 4-(1,1- | Lilial ® | 0.00444000 |
| | dimethylethyl)-α-methyl- | | |
| 86803-90-9 | 4,7-Methano-1H-indene-2- | Scentenal ® | 0.00439000 |
| | carboxaldehyde, octahydro-5- | | |
| | methoxy- | | |
| 68991-97-9 | 2-Naphthalenecarboxaldehyde, | Melafleur | 0.00436000 |
| | 1,2,3,4,5,6,7,8-octahydro-8,8- | | |
| | dimethyl- | | |
| 18871-14-2 | Pentitol, 1,5-anhydro-2,4- | Jasmal | 0.00434000 |
| | dideoxy-2-pentyl-, 3-acetate | | |
| 58567-11-6 | Cyclododecane, | Boisambren Forte | 0.00433000 |
| | (ethoxymethoxy)- | | |
| 94400-98-3 | Naphth[2,3-b]oxirene, | Molaxone | 0.00425000 |
| | 1a,2,3,4,5,6,7,7a-octahydro- | | |
| | 1a,3,3,4,6,6-hexamethyl-, | | |
| | (1aR,4S,7aS)-rel- | | |
| 79-69-6 | 3-Buten-2-one, 4-(2,5,6,6- | alpha-Irone | 0.00419000 |
| | tetramethyl-2-cyclohexen-1-yl)- | | |
| 65442-31-1 | Quinoline, 6-(1-methylpropyl)- | Iso Butyl Quinoline | 0.00408000 |
| 87731-18-8 | Carbonic acid, 4-cycloocten-1-yl | Violiff | 0.00401000 |
| | methyl ester | | |
| 173445-65-3 | 1H-Indene-5-propanal, 2,3- | Hivernal (A-isomer) | 0.00392000 |
| | dihydro-3,3-dimethyl- | | |
| 23911-56-0 | Ethanone, 1-(3-methyl-2- | Nerolione | 0.00383000 |
| | benzofuranyl)- | | |
| 52474-60-9 | 3-Cyclohexene-1- | Precyclemone B | 0.00381000 |
| | carboxaldehyde, 1-methyl-3-(4- | | |
| | methyl-3-penten-1-yl)- | | |
| 139539-66-5 | 6-Oxabicyclo[3.2.1]octane, 5- | Cassifix | 0.00381000 |
| | methyl-1-(2,2,3-trimethyl-3- | | |
| | cyclopenten-1-yl)- | | |
| 80858-47-5 | Benzene, [2-(cyclohexyloxy) | Phenafleur | 0.00380000 |
| | ethyl]- | | |
| 32764-98-0 | 2H-Pyran-2-one, tetrahydro-6- | Jasmolactone | 0.00355000 |
| | (3-penten-1-yl)- | | |
| 78417-28-4 | 2,4,7-Decatrienoic acid, ethyl | Ethyl 2,4,7- | 0.00353000 |
| | ester | decatrienoate | |
| 140-26-1 | Butanoic acid, 3-methyl-, 2- | Beta Phenyl Ethyl | 0.00347000 |
| | phenylethyl ester | Isovalerate | |
| 105-90-8 | 2,6-Octadien-1-ol, 3,7-dimethyl-, | Geranyl Propionate | 0.003360000 |
| | 1-propanoate, (2E)- | | |
| 41816-03-9 | Spiro[1,4-methanonaphthalene- | Rhubofix ® | 0.00332000 |
| | 2(1H), 2′-oxirane], 3,4,4a,5,8,8a- | | |
| | hexahydro-3′,7-dimethyl- | | |
| 7070-15-7 | Ethanol, 2-[[(1R,2R,4R)-1,7,7- | Arbanol | 0.00326000 |
| | trimethylbicyclo[2.2.1]hept-2-yl] | | |
| | oxy]-, rel- | | |
| 93-29-8 | Phenol, 2-methoxy-4-(1-propen- | Iso Eugenol Acetate | 0.00324000 |
| | 1-yl)-, 1-acetate | | |
| 476332-65-7 | 2H-Indeno[4,5-b]furan, | Amber Xtreme | 0.00323000 |
| | decahydro-2,2,6,6,7,8,8- | Compound 1 | |
| | heptamethyl- | | |
| 68901-15-5 | Acetic acid, 2-(cyclohexyloxy)-, | Cyclogalbanate | 0.00323000 |
| | 2-propen-1-yl ester | | |
| 107-75-5 | Octanal, 7-hydroxy-3,7- | Hydroxycitronellal | 0.00318000 |
| | dimethyl- | | |
| 68611-23-4 | Naphtho[2,1-b]furan, 9b- | Grisalva | 0.00305000 |
| | ethyldodecahydro-3a,7,7- | | |
| | trimethyl- | | |
| 313973-37-4 | 1,6-Heptadien-3-one, 2- | Pharaone | 0.00298000 |
| | cyclohexyl- | | |
| 137-00-8 | 5-Thiazoleethanol, 4-methyl- | Sulfurol | 0.00297000 |
| 7779-30-8 | 1-Penten-3-one, 1-(2,6,6- | Methyl Ionone | 0.00286000 |
| | trimethyl-2-cyclohexen-1-yl)- | | |
| 127-51-5 | 3-Buten-2-one, 3-methyl-4- | Isoraldeine Pure | 0.00282000 |
| | (2,6,6-trimethyl-2-cyclohexen-1- | | |
| | yl)- | | |
| 72903-27-6 | 1,4-Cyclohexanedicarboxylic | Fructalate ™ | 0.00274000 |
| | acid, 1,4-diethyl ester | | |
| 7388-22-9 | 3-Buten-2-one, 4-(2,2-dimethyl- | Ionone Gamma Methyl | 0.00272000 |
| | 6-methylenecyclohexyl)-3- | | |
| | methyl- | | |
| 104-67-6 | 2(3H)-Furanone, 5- | gamma-Undecalactone | 0.00271000 |
| | heptyldihydro- | (racemic) | |
| 1205-17-0 | 1,3-Benzodioxole-5-propanal, α- | Helional | 0.00270000 |
| | methyl- | | |
| 33704-61-9 | 4H-Inden-4-one, 1,2,3,5,6,7- | Cashmeran | 0.00269000 |
| | hexahydro-1,1,2,3,3- | | |
| | pentamethyl- | | |
| 36306-87-3 | Cyclohexanone, 4-(1- | Kephalis | 0.00269000 |
| | ethoxyethenyl)-3,3,5,5- | | |
| | tetramethyl- | | |
| 97384-48-0 | Benzenepropanenitrile, α- | Citrowanil ® B | 0.00265000 |
| | ethenyl-α-methyl- | | |
| 141-13-9 | 9-Undecenal, 2,6,10-trimethyl- | Adoxal | 0.00257000 |
| 2110-18-1 | Pyridine, 2-(3-phenylpropyl)- | Corps Racine VS | 0.00257000 |
| 27606-09-3 | Indeno[1,2-d]-1,3-dioxin, 4,4a,5, | Magnolan | 0.00251000 |
| | 9b-tetrahydro-2,4-dimethyl- | | |
| 67634-20-2 | Propanoic acid, 2-methyl-, 3a,4, | Cyclabute | 0.00244000 |
| | 5,6,7,7a-hexahydro-4,7- | | |
| | methano-1H-inden-5-yl ester | | |
| 65405-72-3 | 1-Naphthalenol, 1,2,3,4,4a,7,8, | Oxyoctaline Formate | 0.00236000 |
| | 8a-octahydro-2,4a,5,8a- | | |
| | tetramethyl-, 1-formate | | |
| 122-40-7 | Heptanal, 2-(phenylmethylene)- | Amyl Cinnamic | 0.00233000 |
| | | Aldehyde | |
| 103694-68-4 | 2,2-dimethyl-3-(3- | Majantol ® | 0.00224000 |
| | methylphenyl)propan-1-ol | | |
| 13215-88-8 | 2-Cyclohexen-1-one, 4-(2-buten- | Tabanone Coeur | 0.00223000 |
| | 1-ylidene)-3,5,5-trimethyl- | | |
| 25152-85-6 | 3-Hexen-1-ol, 1-benzoate, (3Z)- | Cis-3-Hexenyl Benzoate | 0.00203000 |
| 406488-30-0 | 2-Ethyl-N-methyl-N-(m- | Paradisamide | 0.00200000 |
| | tolyl)butanamide | | |
| 121-33-5 | Benzaldehyde, 4-hydroxy-3- | Vanillin | 0.00194000 |
| | methoxy- | | |
| 77-54-3 | 1H-3a,7-Methanoazulen-6-ol, | Cedac | 0.00192000 |
| | octahydro-3,6,8,8-tetramethyl-, | | |
| | 6-acetate, (3R,3aS,6R,7R,8aS)- | | |
| 76842-49-4 | 4,7-Methano-1H-inden-6-ol, 3a, | Frutene | 0.00184000 |
| | 4,5,6,7,7a-hexahydro-8,8- | | |
| | dimethyl-, 6-propanoate | | |
| 121-39-1 | 2-Oxiranecarboxylic acid, 3- | Ethyl Phenyl Glycidate | 0.00184000 |
| | phenyl-, ethyl ester | | |
| 211299-54-6 | 4H-4a,9-Methanoazuleno[5,6-d]- | Ambrocenide ® | 0.00182000 |
| | 1,3-dioxole, octahydro-2,2,5,8, | | |
| | 8,9a-hexamethyl-, (4aR,5R,7aS, | | |
| | 9R)- | | |
| 285977-85-7 | (2,5-Dimethyl-1,3-dihydroinden- | Lilyflore | 0.00180000 |
| | 2-yl)methanol | | |
| 10094-34-5 | Butanoic acid, 1,1-dimethyl-2- | Dimethyl Benzyl | 0.00168000 |
| | phenylethyl ester | Carbinyl Butyrate | |
| 40785-62-4 | Cyclododeca[c]furan, 1,3,3a,4,5, | Muscogene | 0.00163000 |
| | 6,7,8,9,10,11,13a-dodecahydro- | | |
| 75490-39-0 | Benzenebutanenitrile, α,α,γ- | Khusinil | 0.00162000 |
| | trimethyl- | | |
| 55418-52-5 | 2-Butanone, 4-(1,3-benzodioxol- | Dulcinyl | 0.00161000 |
| | 5-yl)- | | |
| 3943-74-6 | Benzoic acid, 4-hydroxy-3- | Carnaline | 0.00157000 |
| | methoxy-, methyl ester | | |
| 72089-08-8 | 3-Cyclopentene-1-butanol, β,2,2, | Brahmanol ® | 0.00154000 |
| | 3-tetramethyl- | | |
| | 2-Methyl-4-(2,2,3-trimethyl-3- | | |
| | cyclopenten-1-yl)butanol | | |
| 3155-71-3 | 2-Butenal, 2-methyl-4-(2,6,6- | Boronal | 0.00147000 |
| | trimethyl-1-cyclohexen-1-yl)- | | |
| 2050-08-0 | Benzoic acid, 2-hydroxy-, pentyl | Amyl Salicylate | 0.00144000 |
| | ester | | |
| 41199-20-6 | 2-Naphthalenol, decahydro-2,5, | Ambrinol | 0.00140000 |
| | 5-trimethyl- | | |
| 12262-03-2 | ndecanoic acid, 3-methylbutyl | Iso Amyl Undecylenate | 0.00140000 |
| | ester | | |
| 107-74-4 | 1,7-Octanediol, 3,7-dimethyl- | Hydroxyol | 0.00139000 |
| 91-64-5 | 2H-1-Benzopyran-2-one | Coumarin | 0.00130000 |
| 68901-32-6 | 1,3-Dioxolane, 2-[6-methyl-8- | Glycolierral | 0.00121000 |
| | (1-methylethyl)bicyclo[2.2.2] | | |
| | oct-5-en-2-yl]- | | |
| 68039-44-1 | Propanoic acid, 2,2-dimethyl-, | Pivacyclene | 0.00119000 |
| | 3a,4,5,6,7,7a-hexahydro-4,7- | | |
| | methano-1H-inden-6-yl ester | | |
| 106-29-6 | Butanoic acid, (2E)-3,7- | Geranyl Butyrate | 0.00116000 |
| | dimethyl-2,6-octadien-1-yl ester | | |
| 5471-51-2 | 2-Butanone, 4-(4- | Raspberry ketone | 0.00106000 |
| | hydroxyphenyl)- | | |
| 109-42-2 | 10-Undecenoic acid, butyl ester | Butyl Undecylenate | 0.00104000 |

*Vapour Pressures were acquired from Scifinder, which utilises the ACD Software Version 2015, as described in the Test Methods Section.
**Origin: The highly volatile PRMs may be obtained from one or more of the following companies: Firmenich (Geneva, Switzerland), Symrise AG (Holzminden, Germany), Givaudan (Argenteuil, France), IFF (Hazlet, New Jersey), Bedoukian (Danbury, Connecticut), Sigma Aldrich (St. Louis, Missouri), Millennium Speciality Chemicals (Olympia Fields, Illinois), Polarone International (Jersey City, New Jersey), and Aroma & Flavor Specialities (Danbury, Connecticut).
^{§}Torr can be converted into kPa units by multiplying the Torr value by 0.133.

Exemplary highly volatile PRMs selected from the group consisting of the ingredients mentioned in Table 1 are preferred. However, it is understood by one skilled in the art that other highly volatile perfume raw materials, not recited in Table 1, would also fall within the scope of the present invention, so long as they have a vapour pressure greater than 0.001 Torr (>0.00013 kPa) at 25° C. Preferably, the highly volatile perfume raw material is selected from the group consisting of 2,2-dimethyl-3-(3-methylphenyl)propan-1-ol, and 2-phenyl-ethanol.

Furthermore, the fragrance composition comprises a perfume raw material, wherein the perfume raw material further comprises at least one, two, three, four or more low volatility perfume raw materials having a vapour pressure less than 0.001 Torr (<0.00013 kPa) at 25° C., and the low volatility perfume raw material is present in an amount from 0.001 wt % to 50 wt %, preferably less than 40 wt %, or preferably less than 30 wt %, wherein the wt % is relative to the total weight of the perfume raw material. Preferable non-limiting examples of low volatility perfume raw materials having a vapour pressure less than 0.001 Torr (<0.00013 kPa) at 25° C. are listed in Table 2.

TABLE 2
Low Volatility Perfume Raw Materials for Use in the Fragrance Compositions

| CAS | | | Vapor Pressure |
|---|---|---|---|
| Number | Chemical Name | Common Name** | (Torr at 25° C.) *^{§} |
| 1211-29-6 | Cyclopentaneacetic acid, 3-oxo-2- | Methyl jasmonate | 0.00096500 |
| | (2Z)-2-penten-1-yl-, methyl ester, | | |
| | (1R,2R)- | | |
| 28219-60-5 | 2-Buten-1-ol, 2-methyl-4-(2,2,3- | Hindinol | 0.00096100 |
| | trimethyl-3-cyclopenten-1-yl)- | | |
| 93-08-3 | Ethanone, 1-(2-naphthalenyl)- | Methyl beta-naphthyl | 0.00095700 |
| | | ketone | |
| 67633-95-8 | 3-Decanone, 1-hydroxy- | Methyl Lavender | 0.00095100 |
| | | Ketone | |
| 198404-98-7 | Cyclopropanemethanol, 1-methyl- | Javanol ® | 0.00090200 |
| | 2-[(1,2,2-trimethylbicyclo[3.1.0] | | |
| | hex-3-yl)methyl]- | | |
| 121-32-4 | Benzaldehyde, 3-ethoxy-4- | Ethyl vanillin | 0.00088400 |
| | hydroxy- | | |
| 72403-67-9 | 3-Cyclohexene-1-methanol, 4-(4- | Myraldylacetate | 0.00087900 |
| | methyl-3-penten-1-yl)-, 1-acetate | | |
| 28940-11-6 | 2H-1,5-Benzodioxepin-3(4H)-one, | Calone | 0.00083100 |
| | 7-methyl- | | |
| 139504-68-0 | 2-Butanol, 1-[[2-(1,1- | Amber core | 0.00080300 |
| | dimethylethyl)cyclohexyl]oxy]- | | |
| 502847-01-0 | Spiro[5.5]undec-8-en-1-one, 2,2,7, | Spiro[5.5]undec-8-en-1- | 0.00073100 |
| | 9-tetramethyl- | one, 2,2,7,9- | |
| | | tetramethyl- | |
| 2570-03-8 | Cyclopentaneacetic acid, 3-oxo-2- | trans-Hedione | 0.00071000 |
| | pentyl-, methyl ester, (1R,2R)-rel- | | |
| 24851-98-7 (or | Cyclopentaneacetic acid, 3-oxo-2- | Methyl | 0.00071000 |
| 128087-96-7) | pentyl-, methyl ester | dihydrojasmonate or | |
| | | alternatives 1 | |
| 101-86-0 | Octanal, 2-(phenylmethylene)- | Hexyl cinnamic | 0.00069700 |
| | | aldehyde | |
| 365411-50-3 | Indeno[4,5-d]-1,3-dioxin, 4,4a,5,6, | Nebulone | 0.00069200 |
| | 7,8,9,9b-octahydro-7,7,8,9,9- | | |
| | pentamethyl- | | |
| 37172-53-5 | Cyclopentanecarboxylic acid, 2- | Dihydro Iso Jasmonate | 0.00067500 |
| | hexyl-3-oxo-, methyl ester | | |
| 65113-99-7 | 3-Cyclopentene-1-butanol, α,β,2,2, | Sandalore ® | 0.00062500 |
| | 3-pentamethyl- | | |
| 68133-79-9 | Cyclopentanone, 2-(3,7-dimethyl-2, | Apritone | 0.00062000 |
| | 6-octadien-1-yl)- | | |
| 7212-44-4 | 1,6,10-Dodecatrien-3-ol, 3,7,11- | Nerolidol | 0.00061600 |
| | trimethyl- | | |
| 53243-59-7 | 2-Pentenenitrile, 3-methyl-5- | Citronitril | 0.00061500 |
| | phenyl-, (2Z)- | | |
| 134123-93-6 | Benzenepropanenitrile, 4-ethyl-α,α- | Fleuranil | 0.00057600 |
| | dimethyl- | | |
| 77-53-2 | 1H-3a,7-Methanoazulen-6-ol, | Cedrol Crude | 0.00056900 |
| | octahydro-3,6,8,8-tetramethyl-, | | |
| | (3R,3aS,6R,7R,8aS)- | | |
| 68155-66-8 | Ethanone, 1-(1,2,3,5,6,7,8,8a- | Iso Gamma Super | 0.00056500 |
| | octahydro-2,3,8,8-tetramethyl-2- | | |
| | naphthalenyl)- | | |
| 54464-57-2 | Ethanone, 1-(1,2,3,4,5,6,7,8- | Iso-E Super ® | 0.00053800 |
| | octahydro-2,3,8,8-tetramethyl-2- | | |
| | naphthalenyl)- | | |
| 774-55-0 | Ethanone, 1-(5,6,7,8-tetrahydro-2- | Florantone | 0.00053000 |
| | naphthalenyl)- | | |
| 141-92-4 | 2-Octanol, 8,8-dimethoxy-2,6- | Hydroxycitronellal | 0.00052000 |
| | dimethyl- | Dimethyl Acetal | |
| 20665-85-4 | Propanoic acid, 2-methyl-, 4- | Vanillin isobutyrate | 0.00051200 |
| | formyl-2-methoxyphenyl ester | | |
| 79-78-7 | 1,6-Heptadien-3-one, 1-(2,6,6- | Hexalon | 0.00049800 |
| | trimethyl-2-cyclohexen-1-yl)- | | |
| 6259-76-3 | Benzoic acid, 2-hydroxy-, hexyl | Hexyl Salicylate | 0.00049100 |
| | ester | | |
| 93-99-2 | Benzoic acid, phenyl ester | Phenyl Benzoate | 0.00047900 |
| 153859-23-5 | Cyclohexanepropanol, 2,2,6- | Norlimbanol | 0.00046900 |
| | trimethyl-α-propyl-, (1R,6S)- | | |
| 70788-30-6 | Cyclohexanepropanol, 2,2,6- | Timberol | 0.00046900 |
| | trimethyl-α-propyl- | | |
| 68555-58-8 | Benzoic acid, 2-hydroxy-, 3- | Prenyl Salicylate | 0.00045700 |
| | methyl-2-buten-1-yl ester | | |
| 950919-28-5 | 2H-1,5-Benzodioxepin-3(4H)-one, | Cascalone | 0.00045500 |
| | 7-(1-methylethyl)- | | |
| 30168-23-1 | Butanal, 4-(octahydro-4,7- | Dupical | 0.00044100 |
| | methano-5H-inden-5-ylidene)- | | |
| 1222-05-5 | Cyclopenta[g]-2-benzopyran, 1,3,4, | Galaxolide ® | 0.00041400 |
| | 6,7,8-hexahydro-4,6,6,7,8,8- | | |
| | hexamethyl- | | |
| 4602-84-0 | 2,6,10-Dodecatrien-1-ol, 3,7,11- | Farnesol | 0.00037000 |
| | trimethyl- | | |
| 95962-14-4 | Cyclopentanone, 2-[2-(4-methyl-3- | Nectaryl | 0.00036700 |
| | cyclohexen-1-yl)propyl]- | | |
| 4674-50-4 | 2(3H)-Naphthalenone, 4,4a,5,6,7,8- | Nootkatone | 0.00035800 |
| | hexahydro-4,4a-dimethyl-6-(1- | | |
| | methylethenyl)-, (4R,4aS,6R)- | | |
| 3487-99-8 | 2-Propenoic acid, 3-phenyl-, pentyl | Amyl Cinnamate | 0.00035200 |
| | ester | | |
| 10522-41-5 | 2-hydroxy-2-phenylethy acetate | hydroxyphenethyl | 0.00033900 |
| | | acetate | |
| 118-71-8 | 4H-Pyran-4-one, 3-hydroxy-2- | Maltol | 0.00033700 |
| | methyl- | | |
| 128119-70-0 | 1-Propanol, 2-methyl-3-[(1-7,7- | Bornafix | 0.00033400 |
| | trimethylbicyclo[2.2.1]hept-2-yl) | | |
| | oxy]- | | |
| 103614-86-4 | 1-Naphthalenol, 1,2,3,4,4a,5,8,8a- | Octalynol | 0.00033200 |
| | octahydro-2,2,6,8-tetramethyl- | | |
| 7785-33-3 | 2-Butenoic acid, 2-methyl-, (2E)-3, | Geranyl Tiglate | 0.00033200 |
| | 7-dimethyl-2,6-octadien-1-yl ester, | | |
| | (2E)- | | |
| 117933-89-8 | 1,3-Dioxane, 2-(2,4-dimethyl-3- | Karanal | 0.00033100 |
| | cyclohexen-1-yl)-5-methyl-5-(1- | | |
| | methylpropyl)- | | |
| 629-92-5 | Nonadecane | Nonadecane | 0.00032500 |
| 67801-20-1 | 4-Penten-2-ol, 3-methyl-5-(2,2,3- | Ebanol | 0.00028100 |
| | trimethyl-3-cyclopenten-1-yl)- | | |
| 65416-14-0 | Propanoic acid, 2-methyl-, 2- | Maltol Isobutyrate | 0.00028000 |
| | methyl-4-oxo-4H-pyran-3-yl ester | | |
| 28219-61-6 | 2-Buten-1-ol, 2-ethyl-4-(2,2,3- | Laevo Trisandol | 0.00028000 |
| | trimethyl-3-cyclopenten-1-yl)- | | |
| 5986-55-0 | 1,6-Methanonaphthalen-1(2H)-ol, | Healingwood | 0.00027800 |
| | octahydro-4,8a,9,9-tetramethyl-, | | |
| | (1R,4S,4aS,6R,8a5)- | | |
| 195251-91-3 | 2H-1,5-Benzodioxepin-3(4H)-one, | Transluzone | 0.00026500 |
| | 7-(1,1-dimethylethyl)- | | |
| 120-51-4 | Benzoic acid, phenylmethyl ester | Benzyl Benzoate | 0.00025400 |
| 3100-36-5 | 8-Cyclohexadecen-1-one | Cyclohexadecenone | 0.00025300 |
| 65405-77-8 | Benzoic acid, 2-hydroxy-, (3Z)-3- | cis-3-Hexenyl salicylate | 0.00024600 |
| | hexen-1-yl ester | | |
| 4940-11-8 | 4H-Pyran-4-one, 2-ethyl-3- | Ethyl Maltol | 0.00022800 |
| | hydroxy- | | |
| 541-91-3 | Cyclopentadecanone, 3-methyl- | Muskone | 0.00017600 |
| 118-58-1 | Benzoic acid, 2-hydroxy-, | Benzyl salicylate | 0.00017500 |
| | phenylmethyl ester | | |
| 81783-01-9 | 6,8-Nonadien-3-one, 2,4,4,7- | Labienoxime | 0.00017300 |
| | tetramethyl-, oxime | | |
| 25485-88-5 | Benzoic acid, 2-hydroxy-, | Cyclohexyl Salicylate | 0.00017300 |
| | cyclohexyl ester | | |
| 91-87-2 | Benzene, [2-(dimethoxymethyl)-1- | Amyl Cinnamic | 0.00016300 |
| | hepten-1-yl]- | Aldehyde Dimethyl | |
| | | Acetal | |
| 104864-90-6 | 3-Cyclopentene-1-butanol, β,2,2,3- | Firsantol | 0.00016000 |
| | tetramethyl-δ-methylene- | | |
| 224031-70-3 | 4-Penten-1-one, 1-spiro[4.5]dec-7- | Spirogalbanone | 0.00015300 |
| | en-7-yl- | | |
| 134-28-1 | 5-Azulenemethanol, | Guaiyl Acetate | 0.00013400 |
| | 1,2,3,4,5,6,7,8-octahydro-α,α,3,8- | | |
| | tetramethyl-, 5-acetate, (3S,5R,8S)- | | |
| 236391-76-7 | Acetic acid, 2-(1-oxopropoxy)-, 1- | Romandolide ® | 0.00012400 |
| | (3,3-dimethylcyclohexyl)ethyl ester | | |
| 115-71-9 | 2-Penten-1-ol, 5-[(1R,3R,6S)-2,3- | cis-alpha-Santalol | 0.00011800 |
| | dimethyltricyclo[2.2.1.02,6]hept-3- | | |
| | yl]-2-methyl-, (2Z)- | | |
| 107898-54-4 | 4-Penten-2-ol, 3,3-dimethyl-5-(2,2, | Polysantol ® | 0.00011700 |
| | 3-trimethyl-3-cyclopenten-1-yl)- | | |
| 69486-14-2 | 5,8-Methano-2H-1-benzopyran-2- | Florex ® | 0.00011000 |
| | one, 6-ethylideneoctahydro- | | |
| 84697-09-6 | Heptanal, 2-[(4-methylphenyl) | Acalea | 0.00010100 |
| | methylene]- | | |
| 14595-54-1 | 4-Cyclopentadecen-1-one, (Z)- | Exaltenone | 0.00009640 |
| 32388-55-9 | Ethanone, 1-[(3R,3aR,7R,8aS)-2,3, | Vertofix ® | 0.00008490 |
| | 4,7,8,8a-hexahydro-3,6,8,8- | | |
| | tetramethyl-1H-3a,7- | | |
| | methanoazulen-5-yl]- | | |
| 131812-67-4 | 1,3-Dioxolane, 2,4-dimethyl-2-(5,6, | Okoumal ® | 0.00007600 |
| | 7,8-tetrahydro-5,5,8,8-tetramethyl- | | |
| | 2-naphthalenyl)- | | |
| 106-02-5 | Oxacyclohexadecan-2-one | Exaltolide ® | 0.00006430 |
| 141773-73-1 | 1-Propanol, 2-[1-(3,3- | Helvetolide ® | 0.00005790 |
| | dimethylcyclohexyl)ethoxy]-2- | | |
| | methyl-, 1-propanoate | | |
| 63314-79-4 | 5-Cyclopentadecen-1-one, 3- | Delta Muscenone | 0.00005650 |
| | methyl- | | |
| 77-42-9 | 2-Penten-1-ol, 2-methyl-5- | cis-beta-Santalol | 0.00004810 |
| | [(1S,2R,4R)-2-methyl-3- | | |
| | methylenebicyclo[2.2.1]hept-2-yl]-, | | |
| | (2Z)- | | |
| 362467-67-2 | 2H-1,5-Benzodioxepin-3(4H)-one, | Azurone | 0.00004770 |
| | 7-(3-methylbutyl)- | | |
| 28371-99-5 | Ethanone, 1-(2,6,10-trimethyl-2,5, | Trimofix O | 0.00004580 |
| | 9-cyclododecatrien-1-yl)- | | |
| 16223-63-5 | 1H-3a,6-Methanoazulene-3- | Khusimol | 0.00004400 |
| | methanol, octahydro-7,7-dimethyl- | | |
| | 8-methylene-, (3S,3aR,6R,8aS)- | | |
| 10461-98-0 | Benzeneacetonitrile, α- | Peonile | 0.00004290 |
| | cyclohexylidene- | | |
| 50607-64-2 | Benzoic acid, 2-[(2- | Mevantraal | 0.00004070 |
| | methylpentylidene)amino]-, methyl | | |
| | ester | | |
| 29895-73-6 | 5-Hydroxy-2-benzyl-1,3-dioxane | Acetal CD | 0.00004050 |
| 94-47-3 | Benzoic acid, 2-phenylethyl ester | Phenyl Ethyl Benzoate | 0.00003480 |
| 3100-36-5 | Cyclohexadec-8-en-1-one | Globanone ® | 0.00003310 |
| 37609-25-9 | 5-Cyclohexadecen-1-One | Ambretone | 0.00003310 |
| 66072-32-0 | Cyclohexanol, 4-(1,7,7- | Iso Bornyl | 0.00003010 |
| | trimethylbicyclo[2.2.1]hept-2-yl)- | Cyclohexanol | |
| 31906-04-4 | 3-Cyclohexene-1-carboxaldehyde, | Lyral ® | 0.00002940 |
| | 4-(4-hydroxy-4-methylpentyl)- | | |
| 21145-77-7 | Ethanone, 1-(5,6,7,8-tetrahydro- | Musk Plus | 0.00002860 |
| | 3,5,5,6,8,8-hexamethyl-2- | | |
| | naphthalenyl)- | | |
| 21145-77-7 | Ethanone, 1-(5,6,7,8-tetrahydro-3, | Fixolide | 0.00002860 |
| | 5,5,6,8,8-hexamethyl-2- | | |
| | naphthalenyl)- | | |
| 22442-01-9 | 2-Cyclopentadecen-1-one, 3- | Muscenone | 0.00002770 |
| | methyl- | | |
| 109-29-5 | Oxacycloheptadecan-2-one | Silvanone Ci | 0.00002600 |
| 101-94-0 | Benzeneacetic acid, 4- | Para Cresyl Phenyl | 0.00002330 |
| | methylphenyl ester | Acetate | |
| 102-20-5 | Benzeneacetic acid, 2-phenylethyl | Phenyl Ethyl Phenyl | 0.00002300 |
| | ester | Acetate | |
| 118562-73-5 | Cyclododecaneethanol, β-methyl- | Hydroxyambran | 0.00001800 |
| 103-41-3 | 2-Propenoic acid, 3-phenyl-, | Benzyl Cinnamate | 0.00001050 |
| | phenylmethyl ester | | |
| 4707-47-5 | Benzoic acid, 2,4-dihydroxy-3,6- | Veramoss | 0.00001050 |
| | dimethyl-, methyl ester | | |
| 183551-83-9 | Naphtho[2,1-b]furan-6(7H)-one, 8, | Myrrhone | 0.00000977 |
| | 9-dihydro-1,5,8-trimethyl-, (8R)- | | |
| 102-17-0 | Benzeneacetic acid, (4- | Para Anisyl Phenyl | 0.00000813 |
| | methoxyphenyl)methyl ester | Acetate | |
| 120-11-6 | Benzene, 2-methoxy-1- | Benzyl Iso Eugenol | 0.00000676 |
| | (phenylmethoxy)-4-(1-propen-1-yl)- | | |
| 102-22-7 | Benzeneacetic acid, (2E)-3,7- | Geranyl Phenylacetate | 0.00000645 |
| | dimethyl-2,6-octadien-1-yl ester | | |
| 111879-80-2 | Oxacyclohexadec-12-en-2-one, | Habanolide 100% | 0.00000431 |
| | (12E)- | | |
| 87-22-9 | Benzoic acid, 2-hydroxy-, 2- | Phenyl Ethyl Salicylate | 0.00000299 |
| | phenylethyl ester | | |
| 78-37-5 | 2-Propenoic acid, 3-phenyl-, 1- | Linalyl Cinnamate | 0.00000174 |
| | ethenyl-1,5-dimethyl-4-hexen-1-yl | | |
| | ester | | |
| 28645-51-4 | Oxacycloheptadec-10-en-2-one | Ambrettolide | 0.00000139 |
| 123-69-3 | Oxacycloheptadec-8-en-2-one, (8Z)- | Ambrettolide | 0.00000136 |
| 3391-83-1 | 1,7-Dioxacycloheptadecan-8-one | Musk RI | 0.00000057 |
| 68527-79-7 | 7-Octen-2-ol, 8-(1H-indol-1-yl)- | Indolene | 0.000000445 |
| | 2,6-dimethyl- | | |
| 89-43-0 | Methyl 2-[(7-hydroxy-3,7- | Aurantinol | 0.0000000100 |
| | dimethyloctylidene)amino]benzoate | | |
| 54982-83-1 | 1,4-Dioxacyclohexadecane-5,16- | Zenolide | 0.00000000834 |
| | dione | | |
| 105-95-3 | 1,4-Dioxacycloheptadecane-5,17- | Ethylene Brassylate | 0.00000000313 |
| | dione | | |
| 3681-73-0 | Hexadecanoic acid, (2E)-3,7- | Hexarose | 0.00000000300 |
| | dimethyl-2,6-octadien-1-yl ester | | |
| 4159-29-9 | Phenol, 4-[3-(benzoyloxy)-1- | Coniferyl benzoate | 0.00000000170 |
| | propen-1-yl]-2-methoxy- | | |
| 144761-91-1 | Benzoic acid, 2-[(1-hydroxy-3- | Trifone DIPG | 0.00000000093 |
| | phenylbutyl)amino]-, methyl ester | | |

* Vapour Pressures were acquired from Scifinder, which utilises the ACD Software Version 2015, as described in the Test Methods Section.
**Origin: same as above for Table 1.
^{§} Torr can be converted into kPa units by multiplying the Torr value by 0.133.

### Test Methods

The following assays set forth must be used in order that the invention described and claimed herein may be more fully understood.

### Test Method 1: Calculated/Predicted Vapour Pressure of the Perfume Raw Materials

In order to determine the vapour pressure for the pure perfume raw materials, go to the website https://scifinder.cas.org/scifinder/view/scifinder/scifinderExplore.jsf and follow these steps to acquire the predicted vapour pressure.

1. Input the CAS registry number for the particular fragrance material.

2. Select the vapour pressure from the search results.

3. Record the vapour pressure (given in Torr at 25° C.). SciFinder uses Advanced Chemistry Development (ACD/Labs) Software Version 2015 (or preferably the latest version update) to calculate a predicted vapour pressure for the particular pure material. If the CAS number for the particular PRM is unknown or does not exist, you can utilize the ACD/Labs reference program to directly determine the vapour pressure. Vapour Pressure is expressed in Torr, wherein 1 Torr is equal to 0.133 kilopascal (kPa).

### Test Method 2: Isoteniscope Vapour Pressure

Prior to beginning the isoteniscope measurements, all ionic liquids are purified by vacuum evaporation to remove the last traces of volatile impurities and water. The PRMs are dried over molecular sieves. All mixtures are prepared gravimetrically. Samples with PRMs and ionic liquids are checked visually for miscibility prior to use. Any samples found to contain more than a single phase are not measured in this method, which typically can occur at higher levels of PRMs (e.g., 0.6 and 0.8 mole fractions).

The Isoteniscope Vapour Pressure Test Method used to experimentally determine the vapour pressure of the pure perfume raw materials and the perfume raw materials in combination with the ionic liquids is a modified version of ASTM D2879-10 wherein the following alterations are made:

### Section 6: Apparatus

6.2: in place of a constant-temperature air bath, a constant-temperature silicone oil bath is used.
6.3: a hot plate with temperature sensor (IKA RCT basic) and mercury thermometer is used.

6.4: a vacuum pump Edwards model RV8 is used.

6.5: a Schlenk manifold is used.

6.6: an Edwards APG100 active pirani vacuum gauge is used.

6.7: an active digital controller Edwards (output of pressure gauge) is used.

6.10: air is used in place of nitrogen throughout.

6.12: the alcohol lamp is substituted for warming by holding via in the palm of the hands.

### Section 8: Procedure

Add to the isoteniscope a quantity of sample to fill the reservoir and have a similar level in the U-bend. Attach the isoteniscope to the Schlenk manifold and evacuate the bulb system and isoteniscope to 0.1 Torr (0.013 kPa). The pressure is maintained to degas the system. Expose the material to a continuous vacuum for several minutes with gentle warming (e.g., rubbing by hand) and tilting of the isoteniscope to spread out the material.

Place the filled isoteniscope vertically into the oil bath and allow to equilibrate. The method used herein uses a lower pressure ((0.1 Torr; 0.013 kPa) rather than the 1 Torr (0.13 kPa) in ASTM D2879-10) and longer exposure time (5 minutes rather than 1 min in ASTM D2879-10) to degas the sample. The U-tube levels are adjusted by controlling the strength of the vacuum rather than by adding nitrogen as in ASTM D2879-10. Repeat measurements at intervals of 5K rather than 25K.

Vapour pressures for the pure PRMs and for the mixtures of ionic liquid and PRM are reported as described in section 9.2 of the ASTM D2879-10 method. The measured vapour pressures are used to calculate the activity coefficients as specified herein.

### Test Method 3: Gas-Phase Infrared Spectroscopy Method

This method determines the relative gas-phase concentration (rc) of a given volatile material in a composition. In particular, the method correlates the relative gas-phase concentrations of a PRM and the IR absorbances of its vapour phase in a gas-phase IR cell. Infrared spectroscopy is well known analytical technique with details provided in references such as Williams, D. H., Fleming, I., & Pretsch, E. (1989). Spectroscopic Methods. Organic Chemistry, (1989); Skoog, D. A., & West, D. M. (1980). Principles of instrumental analysis (Vol. 158). Philadelphia: Saunders College; and Alpert, N. L., Keiser, W. E., & Szymanski, H. A. (2012). IR: theory and practice of infrared spectroscopy. Springer Science & Business Media.

The method requires the equipment as listed in Table 3.

TABLE 3
Equipment

| Equipment | Supplier |
|---|---|
| IR gas cell: 8 metres path-length Specac Cyclone C5 gas | Specac |
| cell | |
| Heating mantle: Heating jacket and 4000 series temperature | Specac |
| controller | |
| Spectrometer: Spectrum 100 FT-IR | Perkin Elmer |

The method includes the following steps:

Step 1—Registration of the Background

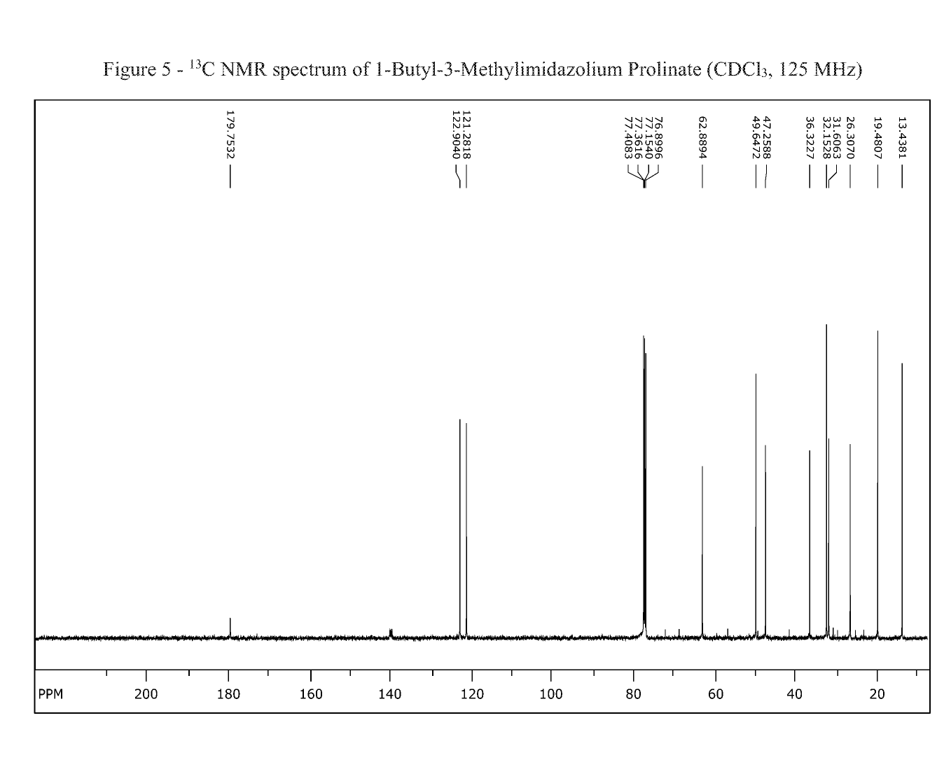

This step is to be completed before each new sample measurement to evacuate all impurities.
a) The IR gas cell is heated with a heating mantle, under vacuum (VP: 675 Torr/90 kPa), to 150° C. and held for 25 mins at that temperature.
b) The IR gas cell is cleaned by being flushed with N₂ gas for another 20 mins while keeping the temperature of the system at 150° C. with the heating mantle.
c) After the cleaning step, the gas phase is checked by running an IR scan of the background in the evacuated IR gas cell in order to determine if there is any residual PRM at the characteristic wavenumber of interest. See FIG. 5 for a typical trace. If a signal is detected, i.e. if the baseline is not flat, the cleaning procedure is repeated until no signal is detected.

Any other volatile component which might affect the spectra has been registered in the background so it can be taken into account in the quantification of the PRM signal.

Step 2—Identification of the Characteristic Peak for a Given Perfume Raw Material

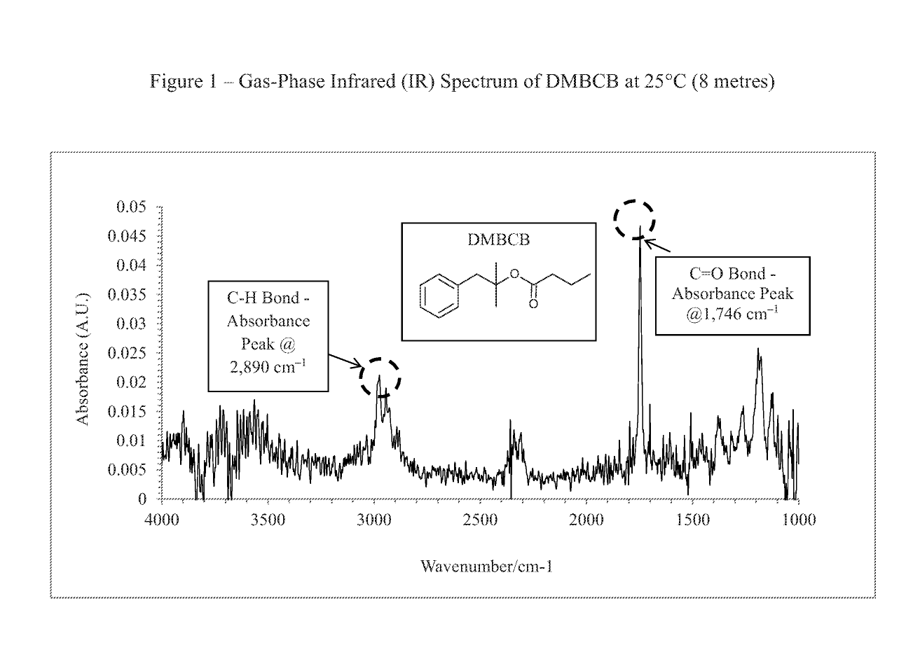

The identification of the fingerprint of the PRM is done by running a mid-IR scan of the vapour phase of the pure PRM in the temperature range of 30-100° C. under vacuum (VP: 675 Torr/90 kPa). The mid-IR range is between about 4,000 and 400 cm⁻¹. See FIG. 1 for an example IR spectrum for a PRM (e.g., dimethyl benzyl carbinyl butyrate (DMBCB)). With continued reference to FIG. 1, the wavenumber is given on the x-axis and the absorbance intensity in Absorbance Unit (A.U.) on the y-axis.

Carbon dioxide in the atmosphere external to the cell is seen in a broad structure band at about 2,400 cm⁻¹, water exhibits a vibrational-rotational spectrum, with rotational fine structure, from 4,000-3,500 cm⁻¹ and a bending mode at 2,000 and the Calcium Floride is observed at less than 1,500 cm⁻¹. This gives the method two useful analytical regions of the spectrum at 2,000-1,500 cm⁻¹ and 3,000-2,500 cm⁻¹. For dimethyl benzyl carbonyl butyrate (“DMBCB”), the CO stretch can be seen clearly with an absorbance peak at 1,746 cm⁻¹ and a CH stretch with an absorbance peak at 2,890 cm⁻¹. In this instance the 1,746 cm⁻¹ peak is the cleanest peak to work with. Some characteristic wavenumbers for common bonds in PRMs are set out in Table 4.

TABLE 4
Wavenumbers for Common Bonds in PRMs

| Bonds | Absorption Region/cm⁻¹ |
|---|---|
| C—C, C—O, C—N | 1,300-800 |
| C═C, C═O, C═N, N═O | 1,900-1,500 |
| C≡C, C≡N | 2,300-2,000 |
| C—H, N—H, O—H | 3,800-2,700 |

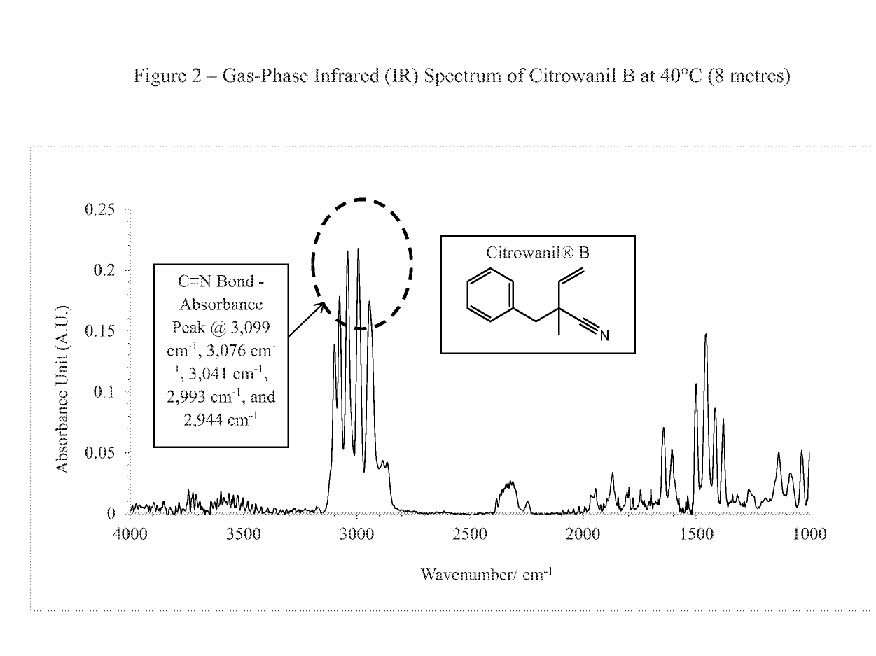

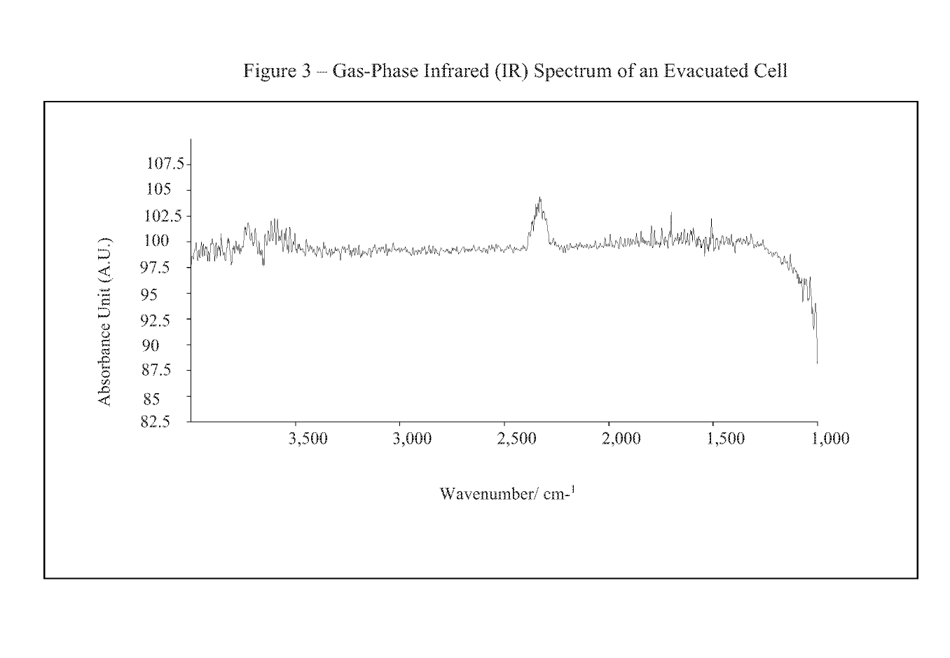

Other example of PRMs include, such as, Citrowanil® B will have a cyanide stretch at 3,099 cm⁻¹, 3,076 cm⁻¹, 3,041 cm⁻¹, 2,993 cm⁻¹, and 2,944 cm⁻¹ (See FIG. 2).

Step 3—Identification of the Absence of Characteristic Peak for Unperfumed Ionic Liquid

The fingerprint of the neat ionic liquid is obtained by running an IR scan of its vapour phase at 25° C. or 40° C. At this temperature no peaks are detected. If a peak (other than originating with water or carbon dioxide) is detected the ionic liquid has been contaminated and a new clean sample must be used.

Step 4—Characterization of the Gas Phase of Mixtures of Ionic Liquids and PRMs
a) The vial containing the test sample is introduced into the IR gas cell. 0.5 g of the test sample is placed on circular tray (diameter 13 mm). It is placed inside the IR gas cell so that it does not interfere with the IR beam pathway.
b) The IR gas cell is closed and is set up under vacuum (VP: 675 Torr/90 kPa).
c) The system is heated up to a temperature between 25-80° C., preferably 25° C., 30° C., 40° C., 45° C., 55° C., 65° C., and 75° C., and held at that temperature.
d) After 40 mins a spectrum is recorded. A second spectrum is recorded after a further 10 mins. If there is no increase in the intensity of the IR peak (change is less than the standard deviation of the method) then it is deemed that equilibrium has been reached. If this condition is not met the spectrum is recorded every 10 mins until this condition is met. The final spectrum at steady-state is recorded for that sample.

Step 5—Calculation of Relative Gas Phase Concentration

The peak area (Aᵢ) and height (hᵢ) are taken as the difference between the peak background baseline recorded with the evacuated cell (as in Step 1 of this procedure) and the peak recorded with the equilibrated sample of interest in the cell. It is the difference in the absorbance for the evacuated cell and the sample of interest.

The peak area (Aᵢ) and height (hᵢ) are recorded for the characteristic signal for:
a) the pure PRM. This is proportional to the pure PRM gas-phase concentration, cᵢ⁰ and the vapour pressure for the pure PRM, Pᵢ^{0;} and
b) each particular PRM-ionic liquid mixture. This is proportional to the gas-phase concentration and hence the vapour pressure of the PRM in the gas phase.

It is generally preferable to use the peak height rather than peak area but this may vary depending on the PRM and its characteristic spectrum. The relative gas phase concentration for any PRM-ionic liquid mixture is calculated as a ratio of its peak height to that of the pure PRM peak height. Therefore the activity co-efficient (γ) at a given PRM mole concentration can be calculated as follows:

γ_{iX}=rc_{iX}/(Xᵢ rcᵢ⁰)

γ_{iX}=h_{iX}/(Xᵢ*hᵢ⁰)

### Test Method 3: Closed Headspace Gas Chromatography

At the beginning of the measurements, all ionic liquids are purified by vacuum evaporation to remove the last traces of volatile compounds. The PRMs are dried over molecular sieves. All mixtures are prepared gravimetrically. Headspace gas chromatography measurements are carried out with a static apparatus with a headspace sampler from Agilent Technologies. The term “headspace” refers to the vapour space above the liquid sample placed in a vial. The 20 cm³ sealed vials move from the sample tray into an oven, in which the vials are heated to a temperature of 305 K (i.e., 32° C.). After reaching equilibrium between the liquid and the vapour phase, the vapour phase of the respective vial is analyzed by gas chromatography (Agilent Technologies) with a flame ionization detector. Since ionic liquids have no measurable vapour pressure, they do not contribute to the gas phase, and hence to the total pressure.

### Test Method 4: Olfactory Tests

In order to show the effect of the ionic liquids on the perception of fragrance profile in a fragrance composition of the present invention, test compositions are made, as described in the Example section, and given to panelists to sample.

At the testing facility, 50 μL samples of the fragrance compositions or the controls are applied to glass slides and placed on a hot plate at 32° C. to represent skin temperature for varying durations. The trained/expert panelists are asked to evaluate the perceived fragrance profile (intensity and/or character) from each pair of samples, i.e., that of the test composition of the present invention vs. the corresponding control, at time 0 and later time points (e.g., 1, 3, 6, 8 and up to 24 hrs post application) as the fragrance profile evolves. Their assessments are recorded. Panelists are selected from individuals who are either trained to evaluate fragrances according to the scales below or who have considerable experience of fragrance evaluation in the industry (i.e., experts).

(a) Fragrance Intensity:

The panelists are asked to give a score on a scale of 0 to 10 for perceived fragrance intensity according to the odor intensity scale set out in Table 5 herein below.

TABLE 5
Odour Intensity Scale

| Score | Fragrance Intensity |
|---|---|
| 0 | Not detectable |
| 2 | Weak |
| 4 | Moderate |
| 6 | Strong |
| 8 | Very Strong |
| 10 | Overpowering |

(b) Fragrance Character:

The panelists provide an expert description of the character of the sample.

### EXAMPLES

The following examples are provided to further illustrate the present invention and are not to be construed as limitations of the present invention, as many variations of the present invention are possible without departing from its spirit or scope.

The structures of the ionic liquids of the present invention can be characterized by various techniques well-known to the skilled person, including for example: ¹H NMR (nuclear magnetic resonance) spectroscopy, ¹³C NMR spectroscopy, halogen analysis and CHN elemental analysis.

Nuclear magnetic resonance (“NMR”) spectroscopy is a spectrometric technique well-known to the skilled person and used herein to characterize the ionic liquids prepared herein.

Mass Spectrometry (“MS”) is a spectrometric technique used herein to quantify the mass to charge ratio of particles or molecules. Two different methods of MS are used: electron spray MS (“ES-MS”) and electron ionization MS (“EI-MS”). ES-MS is used for non-volatile materials such as the ionic liquids. EI-MS is used for volatile materials such as the precursor materials.

### Example 1

### Synthesis of Ionic Liquids

The general method for synthesizing ionic liquids of the present invention consists of: (i) synthesis of a halide precursor; (ii) synthesis of the tertiary amine (iii) quaternisation of an amine using a haloalkane in order to obtain an ionic liquid with a halide anion; and (iv) metathesis (i.e., anion exchange) reaction in order to create the target ionic liquid. This is illustrated in Reaction Scheme 1.

### Reaction Scheme 1

### General Synthesis of Targeted Ionic Liquids

(i) Precursor synthesis step: R—OH+SOCl₂→R—X
(ii) Synthesis of the tertiary amine: R₂—NH+R—X→NR₃
(iii) Quaternisation step: R—X+NR₃→[Cation]X
(iv(a)) Metathesis Step: [Cation]X+M[Anion]→[Cation][Anion]+MX
   where X=F, Cl, Br or I;
   where M=Na or K

Alternatively, the methathesis step can be performed by adding a Brønsted acid of higher acidity than that of the corresponding hydrogen halide. In such case, the methathesis reaction would be preferably defined as a neutralization reaction. For example:
(iv(b)) Neutralization Step: [Cation]X+H[Anion]→[Cation][Anion]+HX
   where X=F, Cl, Br or I

Ionic liquids are formed by combining salts of a cation and an anion (e.g., sodium or potassium salts of the anion and a chloride salt of the cation). Different ionic liquids can be synthesized such that the interactions between the ionic liquids and the solutes (i.e., PRMs) are optimized, preferably to provide for a negative deviation from Raoult's Law. Ionic liquids lend themselves to preparation via combinatorial or high throughput chemistry. The steps shown in the Reaction Scheme 1 are described below in more details.

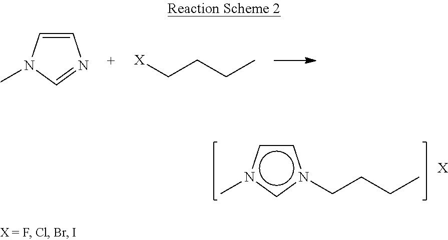

### Step (ii): Synthesis of the Tertiary Amines

Synthesis of 1-butyl-3-methylimidazolium chloride: A mixture of 1-methylimidazole (70.0 g, 0.853 mol), ethanenitrile (50 mL) and 1-chlorobutane (102 g, 1.10 mol) are heated under reflux with vigorous stirring for 48 hrs. 1-chlorobutane (99.5%) is sourced from a commercial supplier (Sigma-Aldrich). Volatile substances are removed in a first step under reduced pressure (ca. 50° C., 20 mbar), and finally, in vacuo (ca. 80° C., 0.01 mbar) during 16 hrs, yielding 1-butyl-3-methylimidazolium chloride ([C₄mim]Cl, 131.8 g, 88.6%) as a pale yellow viscous liquid which crystallizes upon cooling to room temperature.

2-(2-methoxyethoxy)-N-[2-(2-methoxyethoxy)ethyl]-N,N-dimethylethan-1-aminium chloride: A solution of the 2-(2-methoxyethoxy)-N,N-dimethylethan-1-amine (10.00 g, 0.0679 mol), 1-chloro-2-(2-methoxyethoxy)ethane (9.41 g, 0.0679 mol) was added in air to a screw-capped glass tube. The mixture was then stirred (700 rpm) at 50° C. for a week. The orange residue was then washed with ethyl ethanoate (20 cm³×3), cyclohexane (20 cm³×2), and roughly dried using a rotovaporator at 40° C. to leave a hygroscopic orange solid, which was finally dried in vacuo at 40° C. for 3 d (18.50 g, 95% of yield).

TABLE 6
Exemplary Ionic Liquids with Halide Anion from Quaternisation Reaction

| Examle | Chemical Name | Chemical Structure |
|---|---|---|
| Ionic Liquid 1 | 1-butyl-3-methylimidazolium chloride | 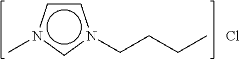 |
| Ionic Liquid 2 | 1-butyl-3-methylimidazolium bromide | 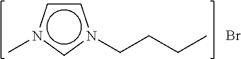 |
| Ionic Liquid 3 | 2-(2-methoxyethoxy)-N-[2-(2- methoxyethoxy)ethyl]-N,N- dimethylethan-1-aminium chloride | 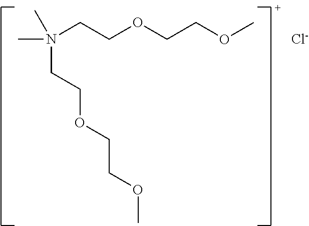 |
| Ionic Liquid 4 | 1-ethanaminium, N,N,N-tris[2- (2-methoxyethoxy)ethyl]- bromide | 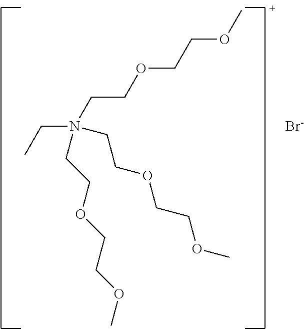 |

### Reaction Scheme 3

### Step (iv): Metathesis Synthesis Reaction of L-Prolinate, Acesulfame and Docusate Ionic Liquids

The synthesis of ionic liquids having anions derived from a-amino-acids can be performed via a series of three consecutive neutralization steps, as shown in the following Reaction Scheme 3, whereby the first step is essentially identical to that described in the Methathesis Step (iiib) above.
[Cation]Cl+H₂SO₄→[Cation][HSO₄]+HCl
[Cation][HSO₄]+Sr[OH]₂→[Cation][OH]+H₂O+Sr[SO₄]
[Cation][OH]+R¹CH(NHR²)COOH→[Cation][R¹CH(NHR²)COO]+H₂O

where R¹CH(NHR²)COOH is an α-amino-acid and R¹ and R² are organic moieties (L-proline represents a particular case where R¹ and R² are part of the same bivalent moiety thus forming a cycle). In the first sub-step, the equilibrium is shifted to the right due to the higher acidic strength of sulfuric acid, whereas the hydrogen chloride by-product is removed by evacuation under reduced pressure. In the second sub-step, the equilibrium is shifted to the right in aqueous solution due to the low solubility product constant of strontium hydroxide, which is readily removed by filtration.

In the following paragraphs, a particular synthetic route resulting in a prolinate ionic liquid is described.

(a) Synthesis of 1-butyl-3-methylimidazolium hydrogensulfate: This preparation was adapted from the procedure by Fraga-Dubreuil et al. (Catal. Commun. 2002, 3, 185). To a solution of 1-butyl-3-methylimidazolium chloride ([C₄mim]Cl, 87.8 g, 0.503 mol) in dichloromethane (50 mL), concentrated sulfuric acid (96%, 51.4 g, 0.503 mol) is added dropwise while keeping the mixture refrigerated in an ice/water bath. The resulting solution is heated under reflux with stirring for 48 hrs. Then, volatiles (mostly containing dichloromethane) are distilled off under reduced pressure (ca. 30° C., 50 mbar), and the hydrogen chloride by-product is similarly removed in vacuo (ca. 90° C., 0.01 mbar) for 16 hrs, yielding 1-butyl-3-methylimidazolium hydrogensulfate ([C₄mim][HSO₄], 118.1 g, 99.4%) as a pale yellow viscous liquid.

(b) Preparation of aqueous 1-butyl-3-methylimidazolium hydroxide: Strontium hydroxide octahydrate (110.14 g, 414.4 mmol) is dissolved in boiling water (approximately 1 L). To the resulting cloudy solution, a solution of 1-butyl-3-methylimidazolium hydrogensulfate ([C₄mim][HSO₄], 93.30 g, 394.9 mmol) in boiling water (100 mL) is added. A white solid immediately forms and precipitates. The mixture is stirred while cooling down to room temperature and then kept at approximately 5° C. overnight (17 hrs). Then, the white solid is removed by filtration. The resulting clear colourless solution is titrated with standard aqueous HCl solution (0.1 N) to determine the [OH]⁻ concentration (0.27 mol kg⁻¹) in the resulting [C₄mim][OH] solution.

(c) Synthesis of 1-butyl-3-methylimidazolium prolinate: To the previously obtained solution (1.321 kg, 357.9 mmol [OH]⁻), a solution of L-proline (41.54 g, 360 8 mmol) in water (100 mL) is added. The resulting solution is concentrated under reduced pressure in a rotary evaporator (50-60° C.) until most of the water is removed, and then placed under high vacuum (70° C.) overnight. The resulting yellowish cloudy viscous liquid is mixed with an acetonitrile/methanol mixture (10:1 by volume, 240 mL), and allowed to settle in the freezer (ca. −20° C.) overnight. The resulting white solid precipitate is removed by filtration. The resulting solution is concentrated in a rotary evaporator (70° C.) until a yellow viscous liquid is obtained, and then dried under high vacuum (70° C.) for 2-3 days. The final ionic liquid ([C₄mim][L-prolinate]) is obtained as a yellowish viscous liquid (86.80 g, 95.7%), with a water content of 156 ppm, and shown in Table 7.

TABLE 7
Exemplary Ionic Liquids with Anion from Metathesis Reaction

| Example | Chemical Name | Chemical Structure |
|---|---|---|
| Ionic Liquid 5 | 1-butyl-3- methylimidazolium prolinate | 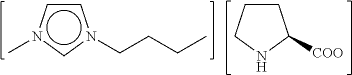 |
| Ionic Liquid 6 | 2-(2-methoxyethoxy)-N-[2- (2-methoxyethoxy)ethyl]- N,N-dimethylethan-1- aminium 6-methyl-3,4- dihydro-1,2,3-oxathiazin-4- one 2,2-dioxide | 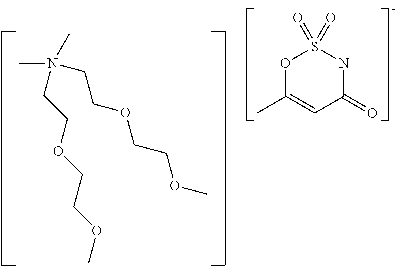 |
| Ionic Liquid 7 | 2-(2-methoxyethoxy)-N-[2- (2-methoxyethoxy)ethyl]- N,N- dimethylethanaminium 1,4- bis(2-ethylhexoxy)-1,4- dioxobutane-2-sulfonate | 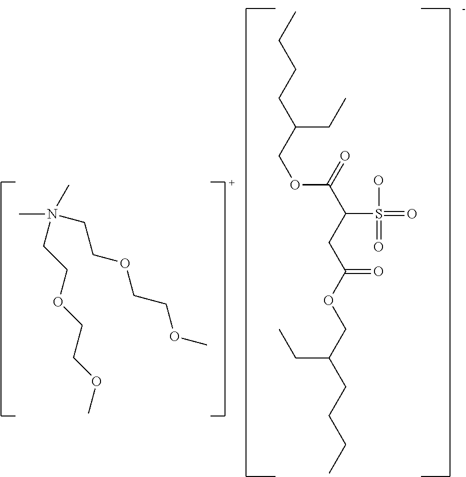 |
| Ionic Liquid 8 | 1-ethanaminium, N,N,N- tris[2-(2-methoxyethoxy) ethyl]-6-methyl-3,4- dihydro-1,2,3-oxathiazin-4- one 2,2-dioxide | 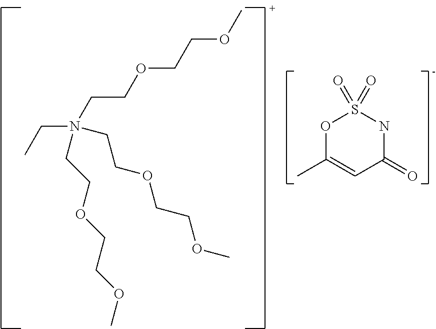 Molecular Weight: 528.66 |
| Ionic Liquid 9 | 1-ethanaminium, N,N,N- tris[2-(2-methoxyethoxy) ethyl]-1,4-bis(2- ethylhexoxy)-1,4- dioxobutane-2-sulfonate | 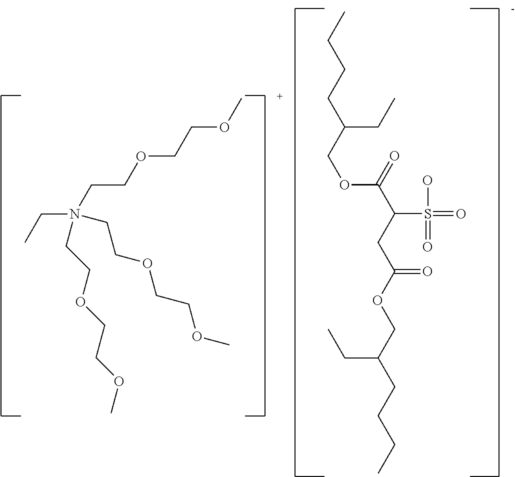 Molecular Weight: 774.06 |

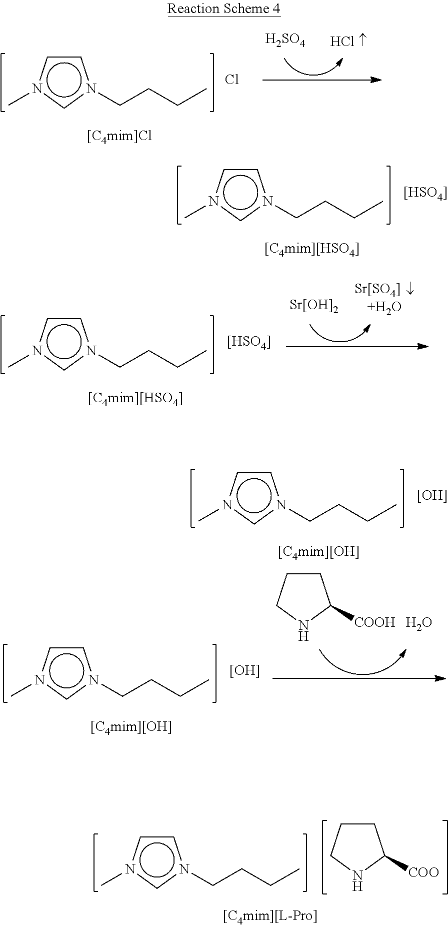

The following Reaction Scheme 4 shows a specific synthetic route from the chloride salt of an imidazolium cation to the final prolinate salt.

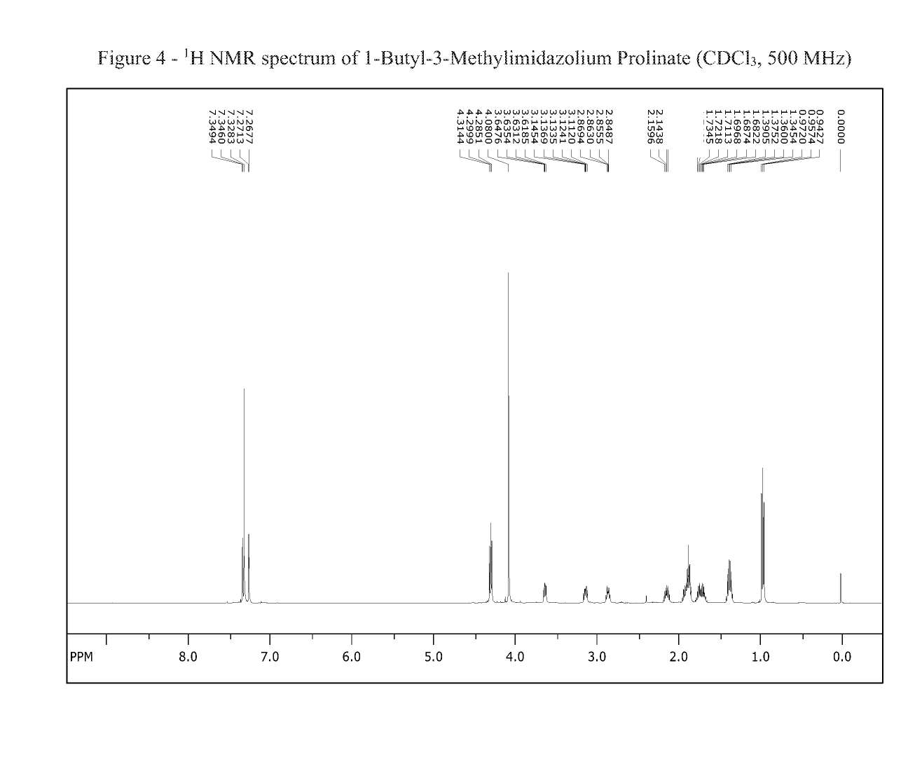

The characterization data for the exemplary ionic liquids are provided in Table 8. The ¹H NMR spectrum of 1-butyl-3-methylimidazolium prolinate (CDCl₃, 500 MHz) is provided in FIG. 4. The ¹³C NMR spectrum of 1-butyl-3-methylimidazolium prolinate (CDCl₃, 125 MHz) is provided in FIG. 5.

TABLE 8
ES-MS and Elemental Analysis Data

| | Elem. | | | | | |
|---|---|---|---|---|---|---|
| Ionic | Anal. | | | | | |
| Liquid | (%) | C | H | N | S | C/N ratio |
| Ionic | Calcd | 61.63 | 9.15 | 16.59 | — | 3.71 |
| Liquid 5 | Measd. | 57.64* | 8.22* | 15.32* | — | 3.76 |
| Ionic | Calcd | 47.87 | 8.03 | 6.57 | 7.52 | 7.29 |
| Liquid 6 | Measd. | 46.77 | 7.96 | 6.88 | 7.98 | 5.86 |
| Ionic | Calcd | 57.20 | 9.75 | 2.08 | 4.77 | 27.5 |
| Liquid 7 | Measd. | 57.61 | 9.88 | 2.41 | 5.16 | 23.9 |
| Ionic | Calcd | 49.98 | 8.39 | 5.30 | 6.07 | 9.43 |
| Liquid 8 | Measd. | 50.11 | 8.89 | 5.66 | 6.46 | 8.85 |
| Ionic | Calcd | 57.41 | 9.77 | 1.81 | 4.14 | 31.7 |
| Liquid 9 | Measd. | 56.96 | 9.53 | 1.55 | 3.89 | 36.7 |

*The deviation observed is due to the absorption of moisture by the sample during handling prior to elemental analysis. Meausured C/N ratio is very close to the calculated ratio.

### Example 2

### Vapour Pressure Measurement

The vapour pressure for the PRM in combination with the ionic liquids is measured using the isoteniscope method as described herein above, and the activity coefficient is determined The results are provided in Tables 9-10.

### Example 2a

IL: Ionic Liquid 5

PRM: Majantol® ^{a}

TABLE 9
Activity Coefficient Measurement for
Single Ionic Liquid Composition

| | Vapour Pressue | Ideal Vapour | Activity |
|---|---|---|---|
| | measured using | Pressure at 100° | coefficient = |
| PRM mole | Isoteniscope | C. according to | Vapour Pressure/ |
| fraction in | method at 100° C. | Raoult's Law | Ideal Vapour |
| Ionic Liquid | (mbar) ^{b} | (mbar) | Pressure |
| 0.2 | 0.8 | 2.8 | 0.3 |
| 0.4 | 0.9 | 5.6 | 0.2 |
| 0.6 | 2.1 | 8.4 | 0.3 |
| 0.8 | 3.7 | 11.2 | 0.3 |

^{a} Majantol ®: 2,2-dimethyl-3-(3-methylphenyl)propan-1-ol (Vapour Pressure = 0.00224 Torr (0.00030 kPa) at 25° C.) (available from Symrise, Germany).
^{b} Average of 2 experiments.

The significant reduction in vapour pressure of Majantol® results in an activity coefficient less than 1 at 0.2, 0.4, 0.6 and 0.8 mole fractions of the PRM. This is likely due to the strong hydrogen-bonding between the prolinate anion and the OH group of Majantol®.

### Example 2b

IL: Ionic Liquid 5

PRM: Phenethyl alcohol (“PEA”)^{a}

TABLE 10
Activity Coefficient Measurement for
Single Ionic Liquid Composition

| | Vapour Pressue | Ideal Vapour | Activity |
|---|---|---|---|
| | measured using | Pressure at 100° | coefficient = |
| PRM mole | Isoteniscope | C. according to | Vapour Pressure/ |
| fraction in | method at 100° C. | Raoult's Law | Ideal Vapour |
| Ionic Liquid | (mbar) | (mbar) | Pressure |
| 0.2 | 0.9 | 3.2 | 0.3 |
| 0.4 | 1.8 | 6.4 | 0.3 |
| 0.6 | 4.0 | 9.5 | 0.4 |
| 0.8 | 10.0 | 12.7 | 0.8 |

^{a} Phenethyl alcohol (Vapour Pressure = 0.0741 Torr (0.00986 kPa) at 25° C.) (available from Firmenich SA, Geneva, Switzerland).

The significant reduction in vapour pressure of PEA results in an activity coefficient less than 1 at 0.2, 0.4, 0.6 and 0.8 mole fractions of the PRM. This is likely due to attractive interactions between the PRM and the ionic liquid anion.

### Example 3

### Relative Gas-Phase Infrared Spectroscopy Measurement

The Relative Gas-Phase Concentration for the PRM in combination with the ionic liquids is measured using the Infra-Red Spectroscopy method as described herein above, and the activity coefficient is determined

### Example 3a

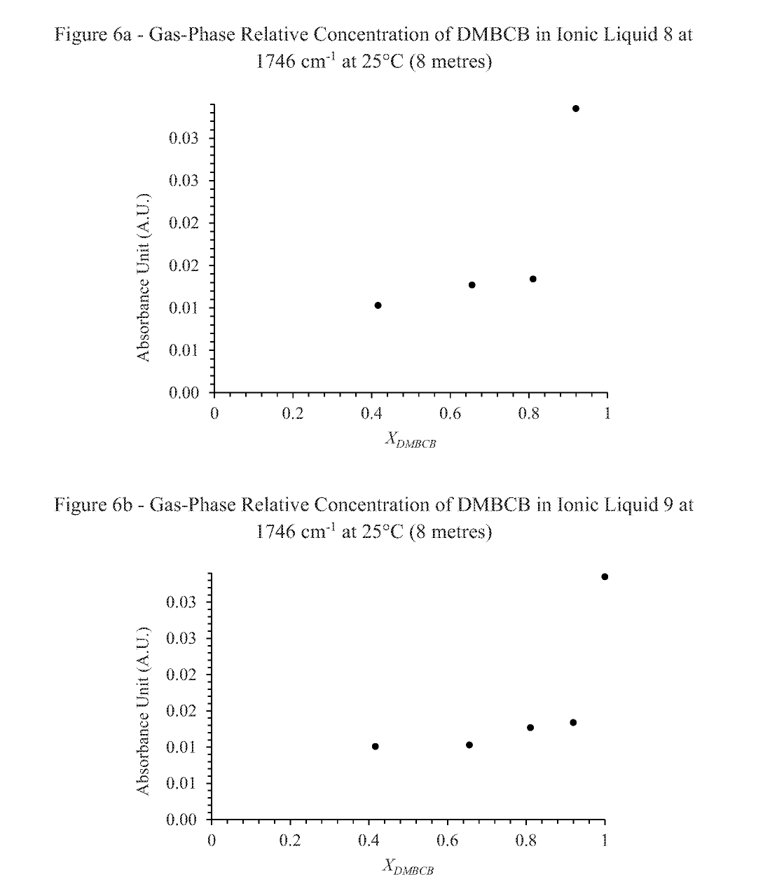

The Gas-Phase relative concentration of DMBCB in Ionic Liquids 8 and 9 at 1746 cm⁻¹ at 25° C. (8 metres) are shown in FIGS. 6a) and 6b), respectively.

### Example 3b

The activity coefficients for DMBCB in Ionic Liquids 8 and 9 (1:1 weight mixture) are provided in Table 11.

TABLE 11
Activity Coefficient Measurement for
a Mixed Ionic Liquid Composition

| PRM ^{a} | Peak Height at | |
|---|---|---|
| molar | 1746 cm⁻¹ at 25° C. | Activity coefficient at 25° C. = |
| Fraction in | using Gas-phase | Peak Height/(molar fraction * |
| Ionic Liquid | IR method (AU) | Peak Height for pure PRM ^{c}) |
| 0.420 | 0.010 ^{b} | 0.0101/(0.42*0.0335) = 0.724 |
| 0.66 | 0.0103 | 0.0103/(0.66*0.0335) = 0.469 |
| 0.810 | 0.0127 | 0.0127/(0.81*0.0335) = 0.468 |
| 0.920 | 0.0134 | 0.0134/(0.92*0.0335) = 0.435 |

^{a} Dimethyl benzyl carbinyl butyrate (VP = 0.00168 Torr (0.000223 kPa) at 25° C.) (International Flavours and Fragrances, New Jersey, USA).
^{b} Average of three measurements.
^{c} Peak Height for pure PRM = 0.0335 A.U. (average of three measurements).

The activity co-efficient of DMBCB at 0.420 0.66, 0.81 and 0.92 mole fractions of DMBCB is less than 1.

### Example 4

### Fragrance Compositions

The following are non-limiting examples of fragrance compositions containing ionic liquids of the present invention. They are prepared by admixture of the components described in Table 12, in the proportions indicated.

TABLE 12
Fragrance Compositions

| | | Fragrance Compositions (wt % ^{a}) | | | | |
|---|---|---|---|---|---|---|
| Ingredients | I | II | III | IV | V | VI |
| PEA ^{b} | 90.0 | 10.0 | 0.0 | 33.5 | 57.0 | 8.0 |
| DMBCB ^{c} | 0.0 | 0.0 | 33.5 | 0.0 | 2.5 | 0.5 |
| Majantol ^{d} | 5.0 | 1.0 | 33.5 | 33.5 | 5.0 | 1.0 |
| Ionic liquid ^{e} | 5 | 89 | 33.0 | 33.0 | 33.0 | 90 |

^{a} wt % relative to the total weight of the composition.
^{b} Phenethyl alcohol (Vapour Pressure = 0.0741 Torr (0.00986 kPa) at 25° C.) (available from Firmenich SA, Generva, Switzerland).
^{c} Dimethyl benzyl carbinyl butyrate (Vapour Pressure = 0.00168 Torr (0.00022 kPa) at 25° C.) (International Flavours and Fragrances (IFF), New Jersey, USA).
^{d} Majantol ®: 2,2-dimethyl-3-(3-methylphenyl)propan-1-ol (Vapour Pressure = 0.00224 Torr (0.00030 kPa) at 25° C.) (available from Symrise, Germany).
^{e} Ionic Liquids 5, 8, 9 or combinations thereof.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A fragrance composition comprising:
(a) from 0.001% to 99.9% by weight of the total fragrance composition of a perfume raw material, wherein the perfume raw material displays a negative deviation from Raoult's Law; and
(b) from 0.01% to 99% by weight of the total fragrance composition of at least one ionic liquid comprising:
(i) an anion; and
(ii) a cation;
wherein the ionic liquid is a liquid at temperatures lower than 100° C.

2. The fragrance composition according to claim 1, wherein the negative deviation from Raoult's Law is determined by the D2879:2010 Standard Test Method (“ASTM D2879 Isoteniscope Method”) or by the Gas-Phase Infrared Spectroscopy Method as described herein.

3. The fragrance composition according claim 1, wherein the perfume raw material displays the negative deviation from Raoult's Law having an activity coefficient less than 1 at a mole fraction between 0.05 and 0.8 of the perfume raw material.

4. The fragrance composition according to claim 1, wherein the cation and the anion are essentially free of the following chemical elements: antimony, barium, beryllium, bromine, cobalt, chromium, fluorine, iodine, lead, nickel, selenium, and thallium.

5. The fragrance composition according claim 1, wherein the perfume raw material comprises at least one highly volatile perfume raw material having a vapour pressure greater than 0.001 Torr (>0.00013 kPa) at 25° C.; and the highly volatile perfume raw material is present in an amount from 0.001 wt % to 99.9 wt %, wherein the wt % is relative to the total weight of the perfume raw materials.

6. The fragrance composition according to claim 5, wherein the perfume raw material comprises at least 2, 3, 4, 5, 6 or more highly volatile perfume raw materials.

7. The fragrance composition according to claim 5, wherein the highly volatile perfume raw material is selected from the group consisting of Table 1 Highly Volatile Perfume Raw Materials and combinations thereof.

8. The fragrance composition according to claim 7, wherein the highly volatile perfume raw material is selected from the group consisting of: 2,2-dimethyl-3-(3-methylphenyl)propan-1-ol, and 2-phenyl-ethanol.

9. The fragrance composition according to claim 5, wherein the perfume raw material further comprises at least one, two, three, four or more low volatility perfume raw materials having a vapour pressure less than 0.001 Torr (<0.00013 kPa) at 25° C., and the low volatility perfume raw material is present in an amount from 0.1 wt % to 50 wt %, wherein the wt % is relative to the total weight of the perfume raw material.

10. The fragrance composition according to claim 1, wherein the anion is independently selected from a compound of formulae (I), (II), (III), (IV), (V), (VI), (VII) or (VIII): 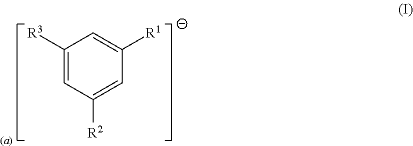
wherein:
R¹ and R³ are independently selected from hydrogen, cyano, hydroxyl, C₁-C₂₀alkyl, C₁-C₂₀alkoxy or C₁-C₂₀alkoxyC₁-C₂₀alkyl;
R² is —R⁴—C(O)O, —R⁴—C(R⁵)CO, —R⁴—C(R⁵)C(O)O, C₁-C₂₀alkyl, C₂-C₂₀alkenyl, C₂-C₂₀alkynyl, C₁-C₂₀alkxoy, C₁-C₂₀alkoxyC₁-C₂₀alkyl, C₃-C₇cycloalkyl, C₃-C₇cycloalkylC₁-C₄alkyl, C₂-C₂₀heterocyclyl, optionally substituted C₆-C₁₀aryl, C₆-C₁₀arylC₁-C₁₀alkyl, C₁-C₁₀heteroaryl;
R⁴ is C₁-C₆alkylene, C₂-C₆alkeneylene, C₂-C₆alkynylene or a direct bond;
R⁵ is hydrogen, hydroxyl, —NH or —N(R^{5a})₂; and
each R^{5a} is independently hydrogen or C₁-C₂₀alkyl; 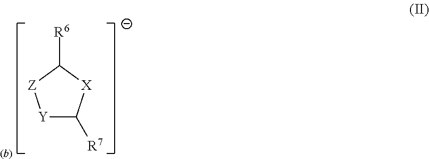
wherein:
X, Y and Z are independently selected from —CH₂—, —NH—, —S—, or —O—;
R⁶ is hydrogen, cyano, hydroxyl, C₁-C₂₀alkyl, C₁-C₂₀alkoxy or C₁-C₂₀alkoxyC₁-C₂₀alkyl;
R^{6a} is C₁-C₆alkylene, C₂-C₆alkeneylene, C₂-C₆alkynylene or a direct bond;
R^{6b} is hydrogen, hydroxyl, —NH or —N(R^{6c})₂;
each R^{6c} is independently hydrogen or C₁-C₂₀alkyl, and
R⁷ is —C(O)O, —R^{6a}—C(R^{6b})CO, —R^{6a}—C(R^{6b})C(O)O, C₁-C₂₀alkyl, C₂-C₂₀alkenyl, C₂-C₂₀alkynyl, C₁-C₂₀alkxoy, C₁-C₂₀alkoxyC₁-C₂₀alkyl, C₃-C₇cycloalkyl, C₃-C₇cycloalkylC₁-C₄alkyl, C₂-C₂₀heterocyclyl, optionally substituted C₆-C₁₀aryl, C₆-C₁₀arylC₁-C₁₀alkyl, C₁-C₁₀heteroaryl; 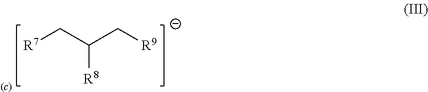
wherein:
R⁷ is —C(R¹⁰)N(R¹¹)₂, —C(O)O, or —S—R¹¹;
R⁸ is hydrogen or C₁-C₂₀alkyl;
R⁹ is —C(O)O or —C(O)N(R¹¹)₂;
R¹⁰ is hydroxyl; and
each R¹¹ is independently hydrogen or C₁-C₂₀alkyl; 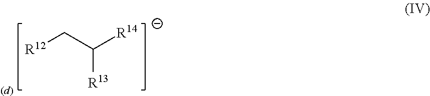
wherein:
R¹² is —C(R¹⁵)₃;
R¹³ is hydrogen or —N(R¹⁶)₂;
R¹⁴ is —R^{14a}—C(O)O;
R^{14a} is C₁-C₆alkylene, C₂-C₆alkeneylene, C₂-C₆alkynylene or a direct bond;
each R¹⁵ is independently selected from hydrogen, C₁-C₂₀alkyl or hydroxyl; and
each R¹⁶ is independently selected from hydrogen or C₁-C₂₀alkyl; 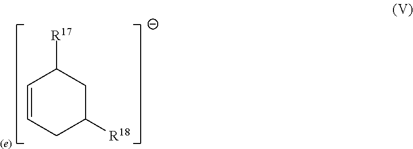
wherein:
R¹⁷ is hydrogen, cyano, hydroxyl, —C(O), C₁-C₂₀alkyl, C₁-C₂₀alkoxy or C₁-C₂₀alkoxyC₁-C₂₀alkyl; and
R¹⁸ is —R^{18a}—C(O)O; —R^{18a}—C(R^{18b})CO, —R^{18a}—C(R^{18b})C(O)O, C₁-C₂₀alkyl, C₂-C₂₀alkenyl, C₂-C₂₀alkynyl, C₁-C₂₀alkxoy, C₁-C₂₀alkoxyC₁-C₂₀alkyl, C₃-C₇cycloalkyl, C₃-C₇cycloalkylC₁-C₄alkyl, C₂-C₂₀heterocyclyl, optionally substituted C₆-C₁₀aryl, C₆-C₁₀arylC₁-C₁₀alkyl, C₁-C₁₀heteroaryl;
R^{18a} is C₁-C₆alkylene, C₂-C₆alkeneylene, C₂-C₆alkynylene or a direct bond;
R^{18b} is hydrogen, hydroxyl, —NH or —N(R^{18c})₂; and
each R^{18c} is independently hydrogen or C₁-C₂₀alkyl; and 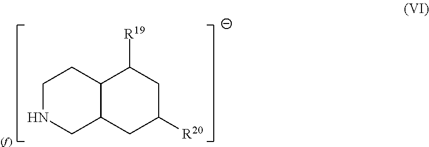
wherein:
R¹⁹ is hydrogen, cyano, hydroxyl, —C(O), C₁-C₂₀alkyl, C₁-C₂₀alkoxy or C₁-C₂₀alkoxyC₁-C₂₀alkyl; and
R²⁰ is —R^{20a}—C(O)O, —R^{20a}—C(R^{20b})CO, —R^{20a}—C(R^{20b})C(O)O, C₁-C₂₀alkyl, C₂-C₂₀alkenyl, C₂-C₂₀alkynyl, C₁-C₂₀alkxoy, C₁-C₂₀alkoxyC₁-C₂₀alkyl, C₃-C₇cycloalkyl, C₃-C₇cycloalkylC₁-C₄alkyl, C₂-C₂₀heterocyclyl, optionally substituted C₆-C₁₀aryl, C₆-C₁₀arylC₁-C₁₀alkyl, C₁-C₁₀heteroaryl;
R^{20a} is C₁-C₆alkylene, C₂-C₆alkeneylene, C₂-C₆alkynylene or a direct bond;
R^{20b} is hydrogen, hydroxyl, —NH or —N(R^{20c})₂; and
each R^{20c} is independently hydrogen or C₁-C₂₀alkyl; 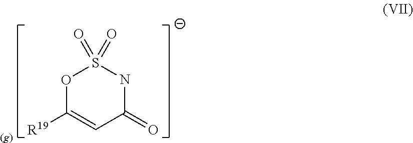
wherein:
R¹⁹ is hydrogen, cyano, alkyl, alkoxy, and alkoxyalkyl; 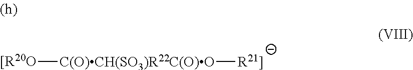
wherein:
R²⁰ and R²¹ are independently selected from the group consisting of alkyl or alkenyl, provided that the alkyl is not substituted with nitro, azido or halide; and
R²² is alkylene, heteroarylene, arylene, or cycloalkylene; and
(i) combinations thereof.

11. The fragrance composition according to claim 10, wherein the anion is independently selected from the group consisting of: 3,5-dihydroxybenzoic acid; 5-hydroxytetrahydrofuran-3-carboxylate; 5-formylcyclohex-3-ene-1-carboxylate; 4-hydroxy-1,3-thiazolidine-2-carboxylate; 3′,5′-dihydroxybiphenyl-3-carboxylate; hydroxy(phenyl)acetate; 5-amino-5-hydroxypentanoate; 4-(3,4-dihydroxyphenyl)butanoate; 5-amino-3-methyl-5-oxopentanoate; 5-hydroxydecahydroisoquinoline-7-carboxylate; 2-amino-3-phenylpropanoate; 2-amino-3-(3-hydroxyphenyl)propanoate; 2-amino-4-hydroxy-4-methylpentanoate; 2-amino-4-hydroxy-4-methylhexanoate; 2-amino-4-(methylsulfanyl)butanoate; L-prolinate; 6 methyl-3,4-dihydro-1,2,3-oxathiazin-4-one 2,2-dioxide; 1,4-bis(2-ethylhexoxy)-1,4-dioxobutane-2-sulfonate; and combinations thereof.

12. The fragrance composition according to claim 1, wherein the cation is independently selected from the group consisting of: 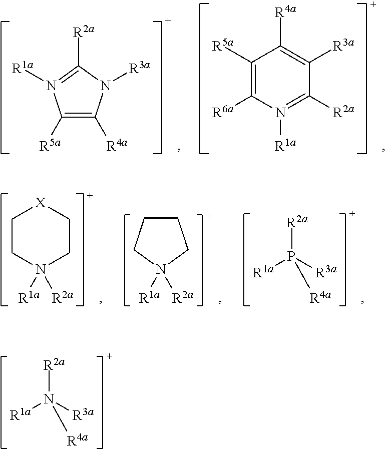
and combinations thereof;
wherein:
X is CH₂ or O;
each R^{1a}, R^{3a}, and R^{4a} are independently selected from hydrogen, C₁-C₂₀alkyl, C₂-C₂₀alkenyl, C₂-C₂₀alkynyl, C₁-C₂₀alkoxy, C₁-C₂₀alkoxyC₁-C₂₀alkyl, C₃-C₇cycloalkyl, C₃-C₇cycloalkylC₁-C₄alkyl, C₂-C₂₀heterocyclyl, C₆-C₁₀aryl, C₆-C₁₀arylC₁-C₁₀alkyl, C₁-C₁₀heteroaryl, halo, haloC₁-C₂₀alkyl, hydroxyl, hydroxylC₁-C₂₀alkyl or —N(R^{6a})₂;
each R^{2a} is independently selected from hydrogen, C₁-C₂₀alkyl, C₁-C₂₀alkenyl, or C₁-C₂₀alkynyl;
each R^{5a} is independently selected from hydrogen, C₁-C₂₀alkyl, C₁-C₂₀alkenyl, C₁-C₂₀ alkynyl, —R^{7a}—OR^{8a}, or —R^{7a}—OR^{7a}—OR^{8a};
each R^{6a} is independently selected from hydrogen, alkyl, alkenyl, alkynyl, haloalkyl, alkoxyalkyl, cycloalkyl, cycloalkylalkyl, aryl, aralkyl, heterocyclyl, heterocyclyalkyl, heteroaryl, or heteroarylalkyl;
each R^{7a} is independently selected from a direct bond, or alkylene chain, or alkenylene chain, or alkynylene chain; and
each R^{8a} is independently selected from a hydrogen, alkyl, alkenyl or alkynyl.

13. The fragrance composition according to claim 12, wherein the cation is independently selected from the group consisting of: 1-butyl-3-methylimidazolium; (N-ethyl-2-(2-methoxyethoxy)-N,N-dimethylethanaminium); 2-(2-ethoxyethoxy)-N-ethyl-N,N-dimethylethanaminium; N-benzyl-N,N-dimethyloctan-1-aminium; N-benzyl-N,N-dimethylnonan-1-aminium; 2-(2-methoxyethoxy)-N-[2-(2-methoxyethoxy)ethyl]-N,N-dimethylethan-1-aminium; 1-ethanaminium, N,N,N-tris[2-(2-methoxyethoxy)ethyl]; and combinations thereof.

14. The fragrance composition according to claim 1, further comprising:
(a) from about 10 wt % to about 80 wt % by weight of the total fragrance composition of a volatile solvent; and
(b) from about 0.1 wt % to about 50 wt % by weight of the total fragrance composition of a low volatility co-solvent or a mixture of low volatility co-solvents.

15. A product comprising a fragrance composition according to claim 1, wherein the product is selected from the group consisting of a perfume, a deodorant, an eau de toilette, an eau de parfum, a cologne, an after shave, a body splash and a body spray.
